SEQUENCE LISTING (165 sequences)

SEQ ID NO: 1 - DNA, 1545 bp, synthetic construct
  source 1..1545: mol_type = other DNA
  sig_peptide 1..54
atgaagctcgccactgctttcaccatccttaccgctgtcttggctgcgcccctccagtcggcctgcaccctccaatccgagactcaccctcctctcacttggcaaaagtgttcgtccggcggcacctgcactcaacagacgggttcggtcgtcatcgacgctaattggcgctggactcacgccactaactcttccactaactgttacgatggcaacacttggtcgtccaccctgtgccctgataacgagacttgtgctaagaactgctgcctcgacggcgccgcttacgcctctacgtatggtgtcaccacctctggtaattccctctccatcggtttcgttacccagtcggctcagaagaacgtcggtgcgcgcctctacttgatggcttctgatactacctaccaggagttcactcttctcggcaacgagttttccttcgatgttgacgtctcccaacttccgtgcggtctcaacggtgctctttatttcgtctcgatggacgcggacggtggcgtctccaaatatcccaccaacaccgctggcgctaagtacggtacgggctattgcgattcccagtgcccccgtgacctcaagtttatcaacggtcaggccaacgttgagggttgggaaccgtcgtcgaataatgcgaacaccggcattggcggccacggctcctgctgttccgaaatggacatttgggaggccaactctatttctgaggccctcacgcctcatccctgcacgaccgtcggtcaggagatttgtgagggtgacggctgcggcggtacctactctgataaccgttatggtggtacttgcgaccccgacggttgcgactggaacccttatcgcctcggtaatacctcgttctatggccctggctcgtccttcaccttggataccaccaagaagctgaccgttgtcacccagttcgaaacgtctggtgccattaaccgttactacgtccagaacggcgtcactttccagcagcccaacgccgagttgggttcctactctggcaatgaacttaacgatgactactgcactgccgaggaggctgagttcggcggctcctctttctctgacaagggtggtttgacccagttcaaaaaggctacctcgggcggcatggtgctcgtcatgtccctttgggatgactactatgcgaacatgctctggctggattccacgtacccgactaatgagacctcgtccactcccggtgctgttcgcggctcctgctctacttcttccggcgttccagctcaggtcgagtctcagtcgcctaacgccaaggtgacgttctctaacatcaagttcggcccaatcggttctaccggcaacccttctggtggtaacccacccggtggtaaccgtggtactaccactacccgtcgacccgcgaccactactggttcttcgcccggtcctacccagtcccactacggtcagtgtggcggtatcggctactcgggccccaccgtttgcgcttcgggcactacttgccaggttttgaacccctactactctcagtgcctttaa

SEQ ID NO: 2 - protein, 514 aa, synthetic construct
  SIGNAL 1..18
MKLATAFTILTAVLAAPLQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 3 - DNA, 1551 bp, synthetic construct
  source 1..1551: mol_type = other DNA
  sig_peptide 1..54
atgaagctcgccactgctttcaccatccttaccgctgtcttggctgcgcccctccagcaggccggtaccgctaccgcggagaaccaccccccccttacttggcaagagtgcactgcgccgggctcctgtacgacgcaaaacggtgctgttgttctcgatgccaactggcgctgggtccacgacgtcaacggctacacgaactgctacaccggtaacacgtgggaccctacctactgccccgatgacgagacctgcgcccagaattgcgcgctcgacggtgccgactacgagggcacctacggcgtcacgtcctctggttcgtccctgaagttgaacttcgtcactggctccaacgtcggctcgcgtttgtaccttctgcaggacgactctacctatcagatcttcaagctccttaaccgcgaattctcgttcgacgtcgatgtttccaacctcccttgcggcctgaacggtgctctttacttcgttgccatggatgccgatggtggcgtctcgaagtatccgaacaataaagccggtgccaagtacggcaccggttattgcgactctcaatgccctcgtgatttgaagtttatcgacggcgaggccaacgtcgagggttggcagccatcgtctaacaacgctaacaccggcatcggtgatcatggctcgtgctgtgccgaaatggacgtttgggaggcgaattccatttccaacgcggtcactccccacccctgtgacaccccgggtcagactatgtgctctggcgacgactgtggtggtacttactccaacgaccgttacgctggtacttgcgaccccgacggttgcgatttcaacccgtatcgcatgggcaatacttcgttctacggccctggtaagattatcgacaccacgaagccctttactgtcgttacccagttcctcacggatgacggcactgatactggtactctctcggagattaagcgcttctatatccagaactccaacgtcattcctcagcccaattctgacatctctggcgtcactggtaattccatcactaccgagttctgcaccgctcaaaaacaggctttcggtgacaccgacgatttctcccagcacggtggtctcgcgaagatgggtgccgctatgcagcagggcatggtcctcgtcatgtccctttgggacgactatgctgctcagatgctctggttggattctgattacccaaccgatgctgatccaaccactcctggcatcgctcgtggcacctgtcccaccgactccggcgtcccttctgacgtcgagtcccagtcccctaactcgtacgttacctactccaatatcaagttcggccccattaactccactttcactgcctctaacccacccggtggtaaccgtggtactaccactacccgtcgacccgcgaccactactggttcttcgcccggtcctacccagtcccactacggtcagtgtggcggtatcggctactcgggccccaccgtttgcgcttcgggcactacttgccaggttttgaacccctactactctcagtgcctttaa

SEQ ID NO: 4 - protein, 516 aa, synthetic construct
  SIGNAL 1..18
MKLATAFTILTAVLAAPLQQAGTATAENHPPLTWQECTAPGSCTTQNGAVVLDANWRWVHDVNGYTNCYTGNTWDPTYCPDDETCAQNCALDGADYEGTYGVTSSGSSLKLNFVTGSNVGSRLYLLQDDSTYQIFKLLNREFSFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYIQNSNVIPQPNSDISGVTGNSITTEFCTAQKQAFGDTDDFSQHGGLAKMGAAMQQGMVLVMSLWDDYAAQMLWLDSDYPTDADPTTPGIARGTCPTDSGVPSDVESQSPNSYVTYSNIKFGPINSTFTASNPPGGNRGTTTTRRPATTTGSSPGPTQSHYGQCGGIGYSGPTVCASGTTCQVLNPYYSQCL

SEQ ID NO: 5 - DNA, 1593 bp, synthetic construct
  source 1..1593: mol_type = other DNA
  sig_peptide 1..54
atgaagctcgccactgctttcaccatccttaccgctgtcttggctgcgcccctcggtacttatactaccgagacccatccttccctgacttggcaaacgtgtacctctgacggttcctgcactacgaatgacggcgaggttgtcattgacgccaattggcgttgggttcactctacttcgtctgccactaactgctacaccggcaatgagtgggatacctcgatctgtacggacgatgtcacctgcgccgcgaactgcgcccttgacggtgctacgtacgaggctacctacggtgtcaccacttctggttctgagttgcgccttaatttcgtcacccagggctcctccaagaacatcggttctcgtctctatctcatgtctgatgattcgaactacgagctctttaagctgctcggccaggaattcacgttcgacgtcgatgtctccaacctcccgtgtggtttgaacggcgccctctatttcgtcgccatggacgcggatggtggtacctcggagtactcgggcaacaaggctggtgcgaaatatggcaccggctactgcgactctcagtgcccccgagacttgaaattcatcaacggcgaggctaactgtgacggctgggagccctcttcgaacaacgttaacactggcgtcggcgaccacggctcctgctgcgccgaaatggacgtctgggaggcgaactccatttccaacgccttcaccgctcacccttgcgactccgtttcccagaccatgtgcgacggtgattcttgcggtggtacgtattctgcctcgggcgatcgttactcgggtacctgtgatcctgatggctgcgactacaatccctatcgtcttggcaacaccgacttctacggtcccggtctcaccgtcgatacgaactccccctttactgtcgttacccagttcatcactgatgacggcacttcttccggtacgctcactgagatcaagcgcctctatgttcagaacggcgaggttatcgctaacggtgcttcgacctacttctcggtcaacggctcttctattacctctgctttctgcgagtcggagaagactttgttcggtgacgagaatgtcttcgataagcacggtggtctggaaggtatgggcgaagccatggctaagggtatggtcctcgtcttgtccctgtgggacgattacgccgcggacatgttgtggcttgactccgactacccggtcaactcctccgcctccaccccaggtgtcgctcgcggcacctgctccaccgactcgggtgtccctgctactgttgaggctgagtccccaaacgcttacgttacttactccaacatcaagttcggtcccattggttccacctattcgtccggctcctcctccggctccggctcttcgtcctcgtcgtcctcgactactactaaggcgacttcgaccacccttaagactacctctaccacttcttccggttcctcctctacgtccgctgcccaggcgtacggccagtgcggcggtcagggctggactggtccaactacctgtgtctcgggctacacctgtacgtacgagaatgcctactactcccagtgcctctaa

SEQ ID NO: 6 - protein, 530 aa, synthetic construct
  SIGNAL 1..18
MKLATAFTILTAVLAAPLGTYTTETHPSLTWQTCTSDGSCTTNDGEVVIDANWRWVHSTSSATNCYTGNEWDTSICTDDVTCAANCALDGATYEATYGVTTSGSELRLNFVTQGSSKNIGSRLYLMSDDSNYELFKLLGQEFTFDVDVSNLPCGLNGALYFVAMDADGGTSEYSGNKAGAKYGTGYCDSQCPRDLKFINGEANCDGWEPSSNNVNTGVGDHGSCCAEMDVWEANSISNAFTAHPCDSVSQTMCDGDSCGGTYSASGDRYSGTCDPDGCDYNPYRLGNTDFYGPGLTVDTNSPFTVVTQFITDDGTSSGTLTEIKRLYVQNGEVIANGASTYFSVNGSSITSAFCESEKTLFGDENVFDKHGGLEGMGEAMAKGMVLVLSLWDDYAADMLWLDSDYPVNSSASTPGVARGTCSTDSGVPATVEAESPNAYVTYSNIKFGPIGSTYSSGSSSGSGSSSSSSSTTTKATSTTLKTTSTTSSGSSSTSAAQAYGQCGGQGWTGPTTCVSGYTCTYENAYYSQCL

SEQ ID NO: 7 - DNA, 1416 bp, synthetic construct
  source 1..1416: mol_type = other DNA
  sig_peptide 1..72
atggtcccctcaatcacacggattgtactggttaacattctgttggcgacgttggttttgggagctgcagtccaggcgtgttcttccgtctggggtcagtgcggcggacagaactggtccggccctacctgctgcgcgtccggttctacctgcgtttactctaacgactattactctcagtgcttgccgggcgcagcctcgtcttcttcgtcgactcgcgctgcttcgacgacctccagagtttcgcccaccacttcgcgttcctcctccgctactccaccacctggctctaccacgactcgtgtcccccctgtcggttctggcaccgccacttactctggtaacccgtttgttggcgttacgccctgggccaacgcctattacgcttctgaggtctcttctttggcgattccctccctcactggcgcgatggctactgccgccgctgctgttgcgaaggtcccgtcgttcatgtggcttgatactttggataagaccccgttgatggagcagacccttgccgacattcgcaccgcgaacaagaacggtggtaactacgccggacagttcgttgtctacgacctgcctgatcgtgattgcgcagcccttgcctccaatggagagtattccattgccgatggtggtgtcgcgaaatataagaactatattgacactattcgccagattgttgtcgagtattcggacatccgcactttgctggtcatcgagcctgactcgcttgctaacttggtcaccaatcttggtacgcccaagtgcgcaaacgcccagtcggcttacctggagtgtatcaactatgccgttactcagcttaatttgcccaacgtcgctatgtacctcgacgccggccacgcaggttggctcggctggcccgctaaccaggaccctgctgctcagctcttcgctaacgtctacaagaacgcttcgtcgccccgcgctctccgtggcctggccactaatgtcgcgaactacaacggttggaatatcacctccccaccctcctacacgcaaggcaacgccgtctacaacgagaagctctacatccatgccatcggtccactcctcgccaaccacggctggtccaacgcgttcttcatcaccgaccagggccgttccggtaagcagcctaccggtcaacagcaatggggcgactggtgcaatgtcatcggtaccggttttggcatccgtccttcggccaacaccggtgactccctccttgactctttcgtctgggtcaagcccggtggcgaatgtgatggcacttccgattcttccgccccgcgcttcgactcccactgcgctctccctgacgctctccagcccgctccacaagccggtgcgtggttccaagcttacttcgtccagcttttgacgaacgctaacccctccttcctctaa

SEQ ID NO: 8 - protein, 471 aa, synthetic construct
  SIGNAL 1..24
MVPSITRIVLVNILLATLVLGAAVQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 9 - DNA, 1278 bp, synthetic construct
  source 1..1278: mol_type = other DNA
  sig_peptide 1..72
atggtcccctcaatcacacggattgtactggttaacattctgttggcgacgttggttttgggagctgcagtccaggccctcgtcgcgctctccccattgctcttctccgccgccacggccttgcctcaagcttccgtcaccccctctccgtcgtcttccgttcctgcttcctctggtccggcccctaccgccaccgctggcggtaacccgttcgagggctacgacttgtatgtcaatccttactacaagtcggaggtcgagtcgctcgctatcccctcgatgactggttcgctcgcggagaaggcctcggctgccgctaacgtcccctccttccactggctcgacaccactgataaggttccacagatgggtgagttccttgaggatatcaagactaagaacgccgctggcgctaacccgcccaccgccggcatcttcgtcgtctacgatttgcccgaccgtgactgcgctgctctggcctccaatggtgagtttttgatttccgacggcggcgttgagaagtataaggcttacattgactctatccgcgaacaggtcgaaaagtactccgatacccagatcattcttgttattgagcctgactccctcgccaatcttgtcactaatcttaacgttcaaaagtgtgcgaacgctcaggacgcctacctcgagtgcaccaactacgcgctcacccagctcaaccttccgaacgtcgcgatgtacctggacgccggtcatgcgggctggctcggctggcccgctaacatcggccccgctgctgaactgtatgcctctgtctacaaaaacgcctcctccccagctgccgttcgaggcctcgctactaacgtcgcgaactataacgccttctctatcgactcctgtccatcttacacgcagggttctaccgtttgcgatgagaagacctacattaataacttcgccccccagctcaaatcggcgggctttgatgcccacttcatcgtcgatactggtcgcaacggtaaccagcccactggtcaatcgcagtggggtgactggtgcaacgtcaagaacactggcttcggcgtccgtcctactaccgacactggtgacgagcttgtcgacgctttcgtctgggttaagcctggtggcgagtcggacggtacctccgatacgtccgctgagcgttatgacgctcactgcggttacgccgatgctttgacgcctgcgcccgaagcgggtacctggttccaggcttatttcgagcagctggtcgagaacgccaatccatctctttaa

SEQ ID NO: 10 - protein, 425 aa, synthetic construct
  SIGNAL 1..24
MVPSITRIVLVNILLATLVLGAAVQALVALSPLLFSAATALPQASVTPSPSSSVPASSGPAPTATAGGNPFEGYDLYVNPYYKSEVESLAIPSMTGSLAEKASAAANVPSFHWLDTTDKVPQMGEFLEDIKTKNAAGANPPTAGIFVVYDLPDRDCAALASNGEFLISDGGVEKYKAYIDSIREQVEKYSDTQIILVIEPDSLANLVTNLNVQKCANAQDAYLECTNYALTQLNLPNVAMYLDAGHAGWLGWPANIGPAAELYASVYKNASSPAAVRGLATNVANYNAFSIDSCPSYTQGSTVCDEKTYINNFAPQLKSAGFDAHFIVDTGRNGNQPTGQSQWGDWCNVKNTGFGVRPTTDTGDELVDAFVWVKPGGESDGTSDTSAERYDAHCGYADALTPAPEAGTWFQAYFEQLVENANPSL

SEQ ID NO: 11 - DNA, 764 bp, synthetic construct
  source 1..764: mol_type = other DNA
  sig_peptide 1..93
atgaaattcatcgttgtcgtcaccactttctcggcgattgcttcggctctcgtccttcctgagggcttcaaacctgaggccgccactgtggctcaaacgatgtgctcccagtacgattctgcctcctccccgccttactccgtcaaccaaaacctctggggtgaataccagggtaccggttctcagtgcgtttacgtcgacaagctctcttcttcgggtgcctcctggcacaccggttggacctggtccggtggtgagggcactgtcaagtcctattccaactccggtgtcacctttaacaagaagctggtctccgatgtctcctcgatccccacctctgttgagtggaagcaagacaacaccaacgtcaatgctgacgtcgcctacgatctcttcaccgccgccaacgttgaccacgcgacgtcctcgggtgactatgagttgatgatttggttggctcgttacggcaacattcagcccatcggtaagcagatcctaccgctactgttggcggcaagtcttgggaagtctggtatggctccactacccaggctggcgcggagcagcgcacttactccttcgtttcggagtcccccatcaattcctattcgggcgacatcaacgcctttttctcctaccttacccagaaccagggtttcccagcttcctctcagtacctcattaatcttcagttcggcaccgaggcgttcactggtggccccgccaccttcactgtcgataactggaccgcctccgtcaactaa

SEQ ID NO: 12 - protein, 254 aa, synthetic construct
  SIGNAL 1..31
MKFIVVVTTFSAIASALVLPEGFKPEAATVAQTMCSQYDSASSPPYSVNQNLWGEYQGTGSQCVYVDKLSSSGASWHTGWTWSGGEGTVKSYSNSGVTFNKKLVSDVSSIPTSVEWKQDNTNVNADVAYDLFTAANVDHATSSGDYELMIWLARYGNIQPIGKQIATATVGGKSWEVWYGSTTQAGAEQRTYSFVSESPINSYSGDINAFFSYLTQNQGFPASSQYLINLQFGTEAFTGGPATFTVDNWTASVN

SEQ ID NO: 13 - DNA, 1033 bp, synthetic construct
  source 1..1033: mol_type = other DNA
  sig_peptide 1..93
atgaaattcatcgttgtcgtcaccactttctcggcgattgcttcggctctcgtccttcctgagggcttcaaacctgaggccgccactgtggctgtcccacacggctccggccataagaagcgtgcctccgtcttcgagtggttcggttctaatgagtctggcgctgaattcggtactaatattccgggtgtctggggcaccgattacatcttccccgatccatctactatctctacgctgattggcaagggtatgaattttttccgtgtccagttcatgatggagcgtctcctgcctgactccatgaccggttcctacgacgaggagtacctcgctaacttgacgaccgtcgtcaaggcggtcaccgatggtggtgcgcacgcccttattgacccgcacaactacggtcgctacaacggcgagatcatttcttcgacttctgacttccaaaccttctggcaaaatctcgccggtcagtataaggacaacgacctttcatgtttgacactaacaacgagtattatgacatggatcaggaccttgtcctcaaccttaaccaggctgccatcaacggtatccgcgctgctggtgcctcccagtacatcttcgtcgagggcaactcgtggactggtgcttggacctgggtcgatgtcaacgataacatgaagaatctcactgaccccgaagataagatcgtctacgagatgcaccagtacctcgactccgatggctcgggcacttcggagacgtgcgtttccggtaccattggtaaggagcgcatcaccgacgccactcagtggctcaaggacaacaagaaggttggtttcattggcgagtatgcgggtggctccaacgacgtttgccgttccgccgtttctggtatgttggagtatatggccaacaacaccgatgtttggaagggcgcgtcctggtgggctgccggcccctggtggggcgactacatcttctcgcttgaaccccccgacggtacgcttacaccggcatgttggatatcttggagacttacctctaa

SEQ ID NO: 14 - protein, 344 aa, synthetic construct
  SIGNAL 1..31
MKFIVVVTTFSAIASALVLPEGFKPEAATVAVPHGSGHKKRASVFEWFGSNESGAEFGTNIPGVWGTDYIFPDPSTISTLIGKGMNFFRVQFMMERLLPDSMTGSYDEEYLANLTTVVKAVTDGGAHALIDPHNYGRYNGEIISSTSDFQTFWQNLAGQYKDNDLVMFDTNNEYYDMDQDLVLNLNQAAINGIRAAGASQYIFVEGNSWTGAWTWVDVNDNMKNLTDPEDKIVYEMHQYLDSDGSGTSETCVSGTIGKERITDATQWLKDNKKVGFIGEYAGGSNDVCRSAVSGMLEYMANNTDVWKGASWWAAGPWWGDYIFSLEPPDGTAYTGMLDILETYL

SEQ ID NO: 15 - DNA, 1324 bp, synthetic construct
  source 1..1324: mol_type = other DNA
  sig_peptide 1..93
atgaaattcatcgttgtcgtcaccactttctcggcgattgcttcggctctcgtccttcctgagggcttcaaacctgaggccgccactgtggctgcctacggtcagtgcggtggtaacggttggaccggtgctacggactgtgtttccggctatcactgcgcttatcagaatgattggtactcccagtgcgtccctggtgccggctccggctccacgtcggccgccgctgccgctgccaccactaaggtcgttacttctgctaaggcttccactactcaagcgccatccaaggcccccgtctccacttcggccgcgtccacttccaaggctgtcgccgctacctccggtaaggttaagtatgctggcgtcaatatcgctggtttcgacttcggtatggacacgaacggcgcttcttccggttcctacgtcgatccgggtactacgggccagaatcagatgaaccacttcgttaaggacgataaactcaacgcctttgtcttcccgtcggctggcagtatcttgtcaactctcagttgggcggcacgttggactccaccttcttcgccaagtacgaccaacagatgacctactgtctcaactccggtgccgctctgtgcatcctcgatctccataattacgctcgttggaacggtcagattgttggcacctctggtggtcccactaatgctcagttcgcgtcggtctggtctcagctcgccaagaagtactcttccaagccaaaggttgccttcgccatcatgaacgaaccccacgatcttcaggacattaacgcttgggcgactaccgtccaggccgccgtcaccgccatccgccaggctggtgccactcaaaacatgattcttcttcccggcgacgactggacccacgctgcgaacttcgttgacaacggttcggccgctgcgctgaacaagatcaccaacctcgatggctcgaagaccaacctggtcttcgacgtccaccagtactccgctccgacggttctggcacctcctcgacttgcgtctcctccgcgtcgaatatcgctggcttcaccaagctcgctacctggctccgctctaacaatcgtcaggcgatgttgaccgaggcgggtggttctaacgatcaatcgtgcttgacggccgtctgcgatgtcctcaactatctcatgaccaactccgatgtctacctcggctggaccggttggtccgctggtatgtttgccactgactacgtcttgtccgaggttccgactggctccgccggctcctacaaggaccaggctattgtcactaagtgtatcgcgggcgttttcaactctaagtaa

SEQ ID NO: 16 - protein, 441 aa, synthetic construct
  SIGNAL 1..31
MKFIVVVTTFSAIASALVLPEGFKPEAATVAAYGQCGGNGWTGATDCVSGYHCAYQNDWYSQCVPGAGSGSTSAAAAAATTKVVTSAKASTTQAPSKAPVSTSAASTSKAVAATSGKVKYAGVNIAGFDFGMDTNGASSGSYVDPGTTGQNQMNHFVKDDKLNAFRLPVGWQYLVNSQLGGTLDSTFFAKYDQQMTYCLNSGAALCILDLHNYARWNGQIVGTSGGPTNAQFASVWSQLAKKYSSKPKVAFAIMNEPHDLQDINAWATTVQAAVTAIRQAGATQNMILLPGDDWTHAANFVDNGSAAALNKITNLDGSKTNLVFDVHQYSDSDGSGTSSTCVSSASNIAGFTKLATWLRSNNRQAMLTEAGGSNDQSCLTAVCDVLNYLMTNSDVYLGWTGWSAGMFATDYVLSEVPTGSAGSYKDQAIVTKCIAGVFNSK

SEQ ID NO: 17 - DNA, 1285 bp, synthetic construct
  source 1..1285: mol_type = other DNA
  sig_peptide 1..93
atgaaattcatcgttgtcgtcaccactttctcggcgattgcttcggctctcgtccttcctgagggcttcaaacctgaggccgccactgtggctcaacagactgtctggggccagtgtggtggcattggctggtccggtcctactaactgtgctccaggctccgcttgttccactttgaacccatattacgcccaatgcatcccgggtgctaccactatcaccacttctactcgtcccccctctggtcccacgacgactactcgtgccacttctacgtcctcctccaccccgcccacttcttcgggcgttcgttttgctggtgtcaacattgctggtttcgactttggctgcacgaccgacggtacttgcgtcacttccaaggtttatcctcccctcaagaatttcaccggctccaacaactaccccgacggcatcggtcagatgcaacacttcgtcaatgaggatggcatgaccatcttccgcttgcccgtcggtggcagtacctcgtcaataacaatctcggtggcaatctcgactcgacctcgatttctaagtatgaccagctcgtccagggttgcctctcgcttggcgcctactgcatcgttgatattcataactacgcccgatggaacggtggtatcattggccagggtggtcccaccaacgctcagttcacctctttgtggtcgcagctcgcgtcgaagtatgcctcccagtcccgtgtttggttcggcattatgaacgagccacacgacgtcaacatcaatacctgggccgccaccgtccaggaggtcgttaccgctatccgtaacgcgggcgccacttcccagttcatctcccttcctggcaatgactggcaatctgctggtgccttcatctcggacggctctgccgcggctctttcccaggtcacgaaccctgacggctccaccaccaacttgatcttcgatgtccacaagtacctggactccgacaactccggtacccacgtgaatgcaccactaacaacatcgatggcgctttctcgccgctcgccacctggctccgccaaaacaaccgccaggcgatcctcaccgagaccggtggtggtaacgtccagtcctgcatccaggacatgtgccagcagattcagtacctgaaccagaactctgatgtttaccttggttacgtcggctggggtgccggctctttcgattccacctacgttttgacggagactcctacgtcttccggtaactcgtggaccgatacctccctcgtctcttcgtgcctggcgcgcaagtaa

SEQ ID NO: 18 - protein, 428 aa, synthetic construct
  SIGNAL 1..31
MKFIVVVTTFSAIASALVLPEGFKPEAATVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTSSGNSWTDTSLVSSCLARK

SEQ ID NO: 19 - DNA, 990 bp, synthetic construct
  source 1..990: mol_type = other DNA
  sig_peptide 1..72
atggtcccctcaatcacacggattgtactggttaacattctgttggcgacgttggttttgggagctgcagtcgagccgcgccaggcgtccgtctctatcgacactaagttcaaggctcacggtaaaaaatacctcggcaacatcggtgatcaatacacccttactaagaattccaagacgccagcgattatcaaggctgactttggcgctctcaccccagagaactccatgaagcgtgacgctactgaaccctctcgtggccagttttcgttctctggttccgattaccttgtcaacttcgcccagtccaacaacaagctcatccgtggtcatacgcttgtttggcactcccagcttccctcctgggttcaagccatcactgataagaacactctcatcggcgtcatgaagaatcacatcaccactgtcatgcagcactacaagggcaagatctatgcctgggacgtcgtcaacgagatcttcaacgaggatggctccctccgtgactcggttttctataaggtcattggtgaggattatgtccgtatcgccttcgagaccgcccgcgctgctgaccctaacgccaagctctacatcaatgactataacttggattcggcttcttaccctaaattgaccggtatggtttcccacgtcaagaagtggattgcggcgggtattccgattgacggcatcggttcgcagacgcacttgtctgcgggtggcggtgccggcatttccggcgctcttaacgccctcgcgggcgctggtaccaaggagattgccgtcaccgaactcgacatcgccgacgcctcgtcgaccgattacgttgaggtcgttgaggcttgcctggaccagcctaagtgcattggtatcactgtctggggtgtcgctgatcctgactcctggcgctcctcgtctacccccctgctcttcgactctaattacaaccccaagcccgcctacttcgccgtcgctaacgctctctaa

SEQ ID NO: 20 - protein, 329 aa, synthetic construct
  SIGNAL 1..24
MVPSITRIVLVNILLATLVLGAAVEPRQASVSIDTKFKAHGKKYLGNIGDQYTLTKNSKTPAIIKADFGALTPENSMKRDATEPSRGQFSFSGSDYLVNFAQSNNKLIRGHTLVWHSQLPSWVQAITDKNTLIGVMKNHITTVMQHYKGKIYAWDVVNEIFNEDGSLRDSVFYKVIGEDYVRIAFETARAADPNAKLYINDYNLDSASYPKLTGMVSHVKKWIAAGIPIDGIGSQTHLSAGGGAGISGALNALAGAGTKEIAVTELDIADASSTDYVEVVEACLDQPKCIGITVWGVADPDSWRSSSTPLLFDSNYNPKPAYFAVANAL

SEQ ID NO: 21 - DNA, 681 bp, synthetic construct
  source 1..681: mol_type = other DNA
  sig_peptide 1..72
atggtcccctcaatcacacggattgtactggttaacattctgttggcgacgttggttttgggagctgcagtcgcgcccaccgagtcgtctgtcgaggttgagaagcgccagaccatcggcccaggtaccggctactccaacggctactattactcctactggaatgacggccacgctggcgtcacttataccaacggtggtggtggttccttcacggttaactggtccaactccggcaacttcgtcggcggtaaaggttggcagccgggcaccaagaacaaggtcatcaacttctctggttcttataaccccaatggcaactcctacctctctatctacggttggtcccgcaaccctttgattgagtactacatcgttgaaaactttggtacctacaacccttccactggtgccaccaagcttggtgaggtcacttctgatggttccgtctacgacatctaccgtactcaacgtgtcaatcagccctcgattattggcaccgctactttctatcagtactggtccgtccgccgaaatcaccgttcttcgggttcggttaacactgctaatcacttcaacgcttgggcctcgcatggcctcacgctgggcacgatggactatcagattgtcgccgttgagggctatttctcctccggttcggcgtcgatcactgtctcttaa

SEQ ID NO: 22 - protein, 226 aa, synthetic construct
  SIGNAL 1..24
MVPSITRIVLVNILLATLVLGAAVAPTESSVEVEKRQTIGPGTGYSNGYYYSYWNDGHAGVTYTNGGGGSFTVNWSNSGNFVGGKGWQPGTKNKVINFSGSYNPNGNSYLSIYGWSRNPLIEYYIVENFGTYNPSTGATKLGEVTSDGSVYDIYRTQRVNQPSIIGTATFYQYWSVRRNHRSSGSVNTANHFNAWASHGLTLGTMDYQIVAVEGYFSSGSASITVS

SEQ ID NO: 23 - DNA, 999 bp, synthetic construct
  source 1..999: mol_type = other DNA
  sig_peptide 1..72
atggtcccgtcgattacccgaatcgtcctcgttaacatcctcctcgccactcttgtcctgggtgcggctgttatcgagccacgccaggctcaggagtctatcaacaagctcatcaaggccaagggtaaactctactacggtacgattacggatcctaatcttcttcagtcccagcaaaacaacgcggttatcaaggctgatttcggtcaagtcaccccggaaaattctatgaagtgggacgctaccgagcctcagcagggcaagttcaacttcggtggcggtgatcaggttgttaacttcgccgcgcagaacggcctcaaggtccgtggccacgctttggtttggcattcccagctcccccagtgggttcataatatcaaggacaagactcaaatgaagaatgctattgagaaccacatcaagaacgtcgctggccacttcaagggtaaggtttatgcttgggacgtcttgaacgagattttcgactgggacggctccctgcgaaaagactctccctttactcaagtccttggcgaggaattcgtcggtattgcttttcgcgccgctcgtgcggcggatcctaacgccaagttgtacatcaatgattactcgatcgacgatcccaacgccgctaagctcaaggcgggcatggttgctcacgtcaaaaagtgggtttcccaaggtattccgatcgatggtatcggttcccagactcaccttgatcccggtgccgctaacggcgtccaggctgccttgcagcagatggcttccactggcgtcaaggaggtcgctatcacggagttggacattcgctctgcgcctgccgccgactatgccaccgtcaccaaggcctgtctcaacgtcccaaagtgcgtcggcattactgtctggggtgtctcggacaaggactcctggcgtaaggaaaaggactcgcttctgttcaacgcccagtaccaggccaaacccgcctataccgcggtcgtcaatgccctgcgctaa

SEQ ID NO: 24 - protein, 332 aa, synthetic construct
  SIGNAL 1..24
MVPSITRIVLVNILLATLVLGAAVIEPRQAQESINKLIKAKGKLYYGTITDPNLLQSQQNNAVIKADFGQVTPENSMKWDATEPQQGKFNFGGGDQVVNFAAQNGLKVRGHALVWHSQLPQWVHNIKDKTQMKNAIENHIKNVAGHFKGKVYAWDVLNEIFDWDGSLRKDSPFTQVLGEEFVGIAFRAARAADPNAKLYINDYSIDDPNAAKLKAGMVAHVKKWVSQGIPIDGIGSQTHLDPGAANGVQAALQQMASTGVKEVAITELDIRSAPAADYATVTKACLNVPKCVGITVWGVSDKDSWRKEKDSLLFNAQYQAKPAYTAVVNALR

SEQ ID NO: 25 - DNA, 804 bp, synthetic construct
  source 1..804: mol_type = other DNA
  sig_peptide 1..72
atggtcccgtcgatcacccgtattgttttggtcaacattctccttgccaccctggtcctcggtgccgctgttgctgaggctattaactacaaccaggattacatcgcctctggtgcgaacgtccagtactccccaaatatggcggccggttctttctccatcaactacaacactcagggcgactttgtcgtcggtcttggttggcagccaggcgacgccaacccgatcacgtactccggctctttctctgcctccggcgttggtattttggcggtctacggttggtcgactaatcctctcgtcgaatattacgtcatggaagtccacgatggttaccagactgctggcacccataagggcactgttactactgacggcggcacctatgacatctgggagcatcaacaggttaaccagccctccatcctgggtacgtccactttcaaccaatacatctctatccgccaatcgccgcgtacgtccggtaccgttaccgtccagaaccacttcaacgcgtgggctcaggccggcatgaatctcggcaccctcaattaccaggtcatggccgtcgagtcgtggtctggttcgggctccggtcagatttcgctttccaagggtaccggcggtggttctactacgacgacccctactggccccacttcgacttctaccgctccttcctccggcggcacgggtgcggcccagtggggccaatgcggtggcattggttggaccggccccaccacctgcgtcgctccctacacctgtaagtatgagaacgcttattattcgcagtgccagtaa

SEQ ID NO: 26 - protein, 267 aa, synthetic construct
  SIGNAL 1..24
MVPSITRIVLVNILLATLVLGAAVAEAINYNQDYIASGANVQYSPNMAAGSFSINYNTQGDFVVGLGWQPGDANPITYSGSFSASGVGILAVYGWSTNPLVEYYVMEVHDGYQTAGTHKGTVTTDGGTYDIWEHQQVNQPSILGTSTFNQYISIRQSPRTSGTVTVQNHFNAWAQAGMNLGTLNYQVMAVESWSGSGSGQISLSKGTGGGSTTTTPTGPTSTSTAPSSGGTGAAQWGQCGGIGWTGPTTCVAPYTCKYENAYYSQCQ

SEQ ID NO: 27 - DNA, 1239 bp, synthetic construct
  source 1..1239: mol_type = other DNA
  sig_peptide 1..72
atggttccatccatcacccgaatcgtcttggtcaacattcttctcgctacccttgtcctcggtgcggccgtccagcttaataccgctgccaaggcggccggcctgctttatttcggtaccgctgtcgacaaccccgacttgtctgactccaagtacctggtcaacctcgaaacggctgacttcggccagatcacgcccgctaacgctatgaagtggcaaccgactgagccatcccaaggttcgtatactttcacgcagggcgatcagatcgcttccctcgcgaagtccaacaacgactatctccgttgccacaatcttgtttggtataatcagctgccctcctacattacgtccggttcgtggactaacgcgactttgatcgcggccctgaaggagcacatcaacggtgtcgtcactcactacaagggtcagtgttacgcttgggacgtcgtcaatgaggccctcaatgaggacggtacttatcgccagaacgttttctaccagcacatcggtgaggcttacattcccatcgcttttgctgcggccgctgccgccgaccctaacgcgaagctttactacaacgattacaacatcgagtacgcgggcgcgaaagccactggcgcccaaggcatcgtcaagctcatccaggccgccggtggtcgcattgatggtgtcggcttgcagtcccactttatcgtcggccaaaccccgtccttggccacccaaaaggctaacatggccgctttcactgctcttggcgtcgatgttgctattactgaactcgatattcgtatgaccctccctgacacctccgctttgcagacccaacagtctactgattatcagacgaccaccactgcctgcgttcagaccaagggctgcgttggtatcaccctgtgggattataccgacaagtattcgtgggttccaggcactttctctggtcagggcgacgcttgcccctgggattccaactacaacaagaaaccggcctactacggcattctcgcgggtctccagtcgggttcgggttcgtcttcctctacctcctcgactactttgattaccacgactactcccactgcctcgtcgtctactacgtctgccaccaccacctccgctacgtctggtgctgcccattggggtcagtgtggcggcattggttggtctggccccaccatctgtgtctccccttacacctgccaggtcctcaatccttactactctcagtgcctctaa

SEQ ID NO: 28 - protein, 412 aa, synthetic construct
  SIGNAL 1..24
MVPSITRIVLVNILLATLVLGAAVQLNTAAKAAGLLYFGTAVDNPDLSDSKYLVNLETADFGQITPANAMKWQPTEPSQGSYTFTQGDQIASLAKSNNDYLRCHNLVWYNQLPSYITSGSWTNATLIAALKEHINGVVTHYKGQCYAWDVVNEALNEDGTYRQNVFYQHIGEAYIPIAFAAAAAADPNAKLYYNDYNIEYAGAKATGAQGIVKLIQAAGGRIDGVGLQSHFIVGQTPSLATQKANMAAFTALGVDVAITELDIRMTLPDTSALQTQQSTDYQTTTTACVQTKGCVGITLWDYTDKYSWVPGTFSGQGDACPWDSNYNKKPAYYGILAGLQSGSGSSSSTSSTTLITTTTPTASSSTTSATTTSATSGAAHWGQCGGIGWSGPTICVSPYTCQVLNPYYSQCL

SEQ ID NO: 29 - DNA, 744 bp, synthetic construct
  source 1..744: mol_type = other DNA
  sig_peptide 1..54
atgaagctcgccactgctttcaccatccttaccgctgtcttggctgcgcccctcggtcaggttcagaacttcaccatcaacggtcagtacaaccagggctttatccttgactattactaccaaaaacagaacaccggtcacttcccgaacgttgctggttggtacgccgaggacctggaccttggctttatttcgccagatcagtacactaccccggacatcgtctgtcataagaatgccgcgcctggtgctatctccgctactgccgccgcgggctccaacatcgtcttccagtggggtcctggcgtctggcctcacccctatggtcctattgttacgtatgtcgtcgagtgctctggctcctgcacgactgtcaataagaataaccttcgttgggttaaaatccaggaagctggcattaattacaacactcaggtctgggcgcagcaagacctcattaaccaaggcaacaagtggaccgtcaagattccgtcgtcgttgcgccccggcaactacgttttccgtcatgagctgttggctgcccacggtgcctcttccgctaacggtatgcagaattaccctcaatgcgtcaacattgcggtcaccggttcgggcacgaaggccctgcccgccggcactcctgcgactcagctttataagccaaccgatcccggcattctcttcaacccctatactacgatcacctcttacactatccccggtccggctctctggcagggctaa

SEQ ID NO: 30 - protein, 247 aa, synthetic construct
  SIGNAL 1..18
MKLATAFTILTAVLAAPLGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 31 - DNA, 1023 bp, synthetic construct
  source 1..1023: mol_type = other DNA
  sig_peptide 1..54
atgaagctcgccactgctttcaccatccttaccgctgtcttggctgcgcccctcggccatattaacgatatcgtcattaacggtgtttggtaccaggcttatgacccgactaccttcccctacgaatctaacccccccatcgtcgtcggttggactgccgctgatctcgacaatggtttcgtctcgcctgatgcttaccagaatcccgacattatctgccataagaatgccaccaatgctaaaggccacgcttccgtcaaggctggtgacaccatcctttttcaatgggtccctgtcccctggccgcacccaggccctatcgtcgattatttggcgaactgcaacggcgactgcgagaccgtcgataagaccaccctcgagttcttcaagatcgatggtgtcggtctcttgtccggcggcgaccccggtacttgggcctctgacgtccttatctctaacaacaatacctgggttgtcaagatccccgacaacctggcccctggtaactacgtcctccgtcacgagatcatcgccctccactcggcgggtcaggctaacggtgcgcagaactacccccagtgcttcaacatcgctgtttcgggctccggctcgctccagccgtctggcgttttgggcactgacttgtatcatgcgacggaccccggtgttctgattaacatttacacctcccccctcaactacattattcccggtccgacggttgtctccggtcttcctacctccgtcgcccagggctcctccgctgctactgccactgcctccgccaccgtcccgggtggtggttctggtcctacttcgcgtactacgactacggcccgcaccactcaagcctcctcgcgtccatcctccacgcctcccgccactacctctgcccctgccggtggtcctacccagaccctttatggccaatgcggcggttccggctactcgggccccacgcgatgtgcgcctccagctacttgttccacccttaatccgtactacgctcagtgtctgaactaa

SEQ ID NO: 32 - protein, 340 aa, synthetic construct
  SIGNAL 1..18
MKLATAFTILTAVLAAPLGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 33 - DNA, 999 bp, Aspergillus niger
  source 1..999: mol_type = genomic DNA
  sig_peptide 1..78
atgaagttcctccaagccaaaggtagcttgctgtcgtctggcatttacctcattgcattagccccctttgtcaacgccaaatgcgctcttccgtcgacatatagttggacttcgaccgatgctctcgcaaccccaaagtctgggtggaccgcactcaaggacttcaccgatgtcgtctctaacggcaaacatattgtctatgcgtccactaccgacacacagggaaattacggctccatgggctttggcgccttttcggactggtcggacatggcatccgctagtcaaacggccacaagcttcagcgctgtagctccaaccttgttctacttccagccgaagagtatctgggttctggcctaccaatggggctccagcactttcacctaccgcacctcccaagatcccaccaatgtcaacggctggtcatccgagcaagctcttttcacgggcaaaatcagcggctcaagtaccggtgccattgatcagactgtgattggtgatgatacgaatatgtatcttttctttgccggcgacaatggcaagatctaccgatccagcatgtctatcaatgacttccccggaagcttcggcagccagtacgaggagatcctcagcggcgcgaccaacgatttgttcgaggcggtccaagtgtacaccgtcgacggcggcgagggtgacagcaagtacctcatgatcgtcgaggcgatcgggtccaccggacatcgttatttccgctccttcacggccagcagtctcggcggagagtggacagcccaggcggcaagtgaagatcaacccttcgcgggcaaagccaacagtggcgccacctggaccgacgacatcagtcatggtgacttggtccgcaacaaccctgatcaaaccatgacggtcgatccttgcaacctccagcttctctaccagggccatgaccccaacagcaatagcgaatacaacctcttgccctggaagccaggagttctcaccttgaagcagtga

SEQ ID NO: 34 - DNA, 999 bp, synthetic construct
  source 1..999: mol_type = other DNA
  sig_peptide 1..78
atgaagttcttacaagccaagggttctttgttgtcttctggtatttacttaattgccttagccccatttgtcaacgccaagtgtgctttaccatctacctatagttggacttctaccgatgctttagccaccccaaagtctggttggaccgccttaaaggacttcaccgatgtcgtctctaacggtaagcatattgtctatgcttccactaccgacacccaaggtaattacggttccatgggttttggtgccttttctgactggtctgacatggcctccgctagtcaaactgccacctctttctctgctgttgctccaaccttgttctacttccaaccaaagagtatctgggttttggcctaccaatggggttcctctactttcacctaccgtacctcccaagatccaaccaatgtcaacggttggtcatccgaacaagctttattcactggtaagatctctggttcaagtaccggtgccattgatcaaactgtcattggtgatgatactaatatgtatttattctttgccggtgacaatggtaagatctacagatcctctatgtctatcaatgacttcccaggttctttcggttctcaatacgaagaaatcttatctggtgctaccaacgatttgttcgaagctgtccaagtctacaccgtcgacggtggtgaaggtgactctaagtacttaatgatcgtcgaagctatcggttccaccggtcatagatatttccgttccttcactgcctctagtttaggtggtgaatggaccgcccaagctgccagtgaagatcaaccattcgctggtaaggccaacagtggtgccacctggaccgacgacatcagtcatggtgacttggtccgtaacaaccctgatcaaaccatgactgtcgatccttgtaacttacaattattataccaaggtcatgacccaaactctaattctgaatacaacttattgccatggaagccaggtgttttaaccttgaagcaatga

SEQ ID NO: 35 - protein, 332 aa, Aspergillus niger
  SIGNAL 1..26
MKFLQAKGSLLSSGIYLIALAPFVNAKCALPSTYSWTSTDALATPKSGWTALKDFTDVVSNGKHIVYASTTDTQGNYGSMGFGAFSDWSDMASASQTATSFSAVAPTLFYFQPKSIWVLAYQWGSSTFTYRTSQDPTNVNGWSSEQALFTGKISGSSTGAIDQTVIGDDTNMYLFFAGDNGKIYRSSMSINDFPGSFGSQYEEILSGATNDLFEAVQVYTVDGGEGDSKYLMIVEAIGSTGHRYFRSFTASSLGGEWTAQAASEDQPFAGKANSGATWTDDISHGDLVRNNPDQTMTVDPCNLQLLYQGHDPNSNSEYNLLPWKPGVLTLKQ

SEQ ID NO: 36 - DNA, 1500 bp, Aspergillus phoenicis
  source 1..1500: mol_type = genomic DNA
  sig_peptide 1..54
atgttctcccgccgaaacctcgtcgccctagggctggcagccaccgtcagcgccggcccctgtgacatctacgaagccggcgacacgccctgcgtagccgcgcacagcaccacccgcgccctatacagctccttcagcggcgccctctaccagctccaacgtggctccgacgacaccaccaccaccatctccccgctcacagccggcggcgtcgccgacgcctccgcccaagacaccttctgtgccaacaccacctgcttgatcaccatcatctacgaccaatccggcaacggcaaccacctgacccaagcacccccgggaggcttcgacggccccgacgtcgacggctacgacaacctcgccagcgccatcggcgcccccgtcaccctcaatggccaaaaggcctacggcgtgttcatgtcccccggcacgggctaccgtaacaacgaagccaccggcaccgccaccggcgacgaacccgaaggcatgtacgccgtcctggacggcacgcactacaacgacgcctgctgcttcgactacggcaacgccgagaccagcagcaccgacactggcgccggccacatggaggccatctacctcgggaacagcaccacctggggctacggcgccggtgacgggccctggatcatggtcgatatggagaacaacctcttctccggtgctgatgagggatataactccggagatccctcgatcagctacagctttgtcactgccgcggtcaagggcggggctgataagtgggctattcgcggtggtaatgctgcctctgggtccctctctacttactacagcggcgctcgcccggattactccggctataaccccatgagcaaggagggcgctatcatcctgggtatcggcggtgacaacagcaacggcgcccagggtaccttctacgagggtgtcatgacctccggctacccgtcggacgatgtcgagaactccgtccaggagaacatcgtggctgcgaaatacgtctccggctcgctggtcagcggcccgtcgttcacctccggagaggtggtctcgctgcgtgtcactacgcccggttacacgacgcggtatattgcgcacactgacaccactgtgaacacgcaggtcgtggacgacgatagttccaccacgctgaaggaggaggctagctggaccgtggtgaccggtctggctaatagtcagtgcttctcgttcgagtcggttgatacccctggtagctatatccggcattacaactttgagttgctgcttaatgccaatgatggcacgaagcagttccatgaggatgctactttctgtcctcaggcgccgttgaatggagagggtacttcgttgagatcgtggagttatccgacgaggtatttcagacattatgataatgtcctgtatgctgctagtaatggcggtgtgcagacgtttgattccaagacatcgttcaataatgatgttagctttgagattgagacggcgtttgcttcgtaa

SEQ ID NO: 37 - DNA, 1500 bp, synthetic construct
  source 1..1500: mol_type = other DNA
  sig_peptide 1..54
atgttctcccgtagaaacttagtcgccttaggtttggccgccaccgtctctgccggtccatgtgacatctacgaagccggtgacactccatgtgttgccgctcactctaccacccgtgccttatactcttccttctctggtgccttataccaattacaaagaggttccgacgacaccaccaccaccatctccccattaaccgccggtggtgtcgccgacgcctccgcccaagacaccttctgtgccaacaccacctgtttgatcaccatcatctacgaccaatccggtaacggtaaccacttgacccaagccccaccaggtggtttcgacggtccagacgtcgacggttacgacaacttagcctctgccatcggtgccccagtcaccttaaatggtcaaaaggcctacggtgtcttcatgtccccaggtactggttacagaaacaacgaagccaccggtaccgccaccggtgacgaaccagaaggtatgtacgccgtcttggacggtactcactacaacgacgcctgttgtttcgactacggtaacgccgaaacctcttctaccgacactggtgccggtcacatggaagccatctacttaggtaactctaccacctggggttacggtgccggtgacggtccatggatcatggtcgatatggaaaacaacttattctccggtgctgatgaaggttataactccggtgatccatctatctcttactcttttgtcactgccgctgtcaagggtggtgctgataagtgggctattcgtggtggtaatgctgcctctggttccttatctacttactactctggtgctcgtccagattactccggttataacccaatgtctaaggaaggtgctatcatcttgggtatcggtggtgacaactctaacggtgcccaaggtaccttctacgaaggtgtcatgacctccggttacccatctgacgatgtcgaaaactccgtccaagaaaacatcgtcgctgctaagtacgtctccggttctttggtctctggtccatctttcacctccggtgaagtcgtctctttgagagtcactactccaggttacactactagatatattgctcacactgacaccactgtcaacactcaagtcgtcgacgacgatagttccaccactttgaaggaagaagcttcttggaccgtcgtcaccggtttggctaatagtcaatgtttctctttcgaatctgttgatacccctggttcttatatcagacattacaactttgaattgttgttaaatgccaatgatggtactaagcaattccatgaagatgctactttctgtcctcaagctccattgaatggtgaaggtacttctttgagatcttggagttatccaactagatatttcagacattatgataatgtcttgtatgctgctagtaatggtggtgtccaaacttttgattccaagacctctttcaataatgatgtttcttttgaaattgaaactgcttttgcttcttaa

SEQ ID NO: 38 - protein, 499 aa, Aspergillus phoenicis
  SIGNAL 1..18
MFSRRNLVALGLAATVSAGPCDIYEAGDTPCVAAHSTTRALYSSFSGALYQLQRGSDDTTTTISPLTAGGVADASAQDTFCANTTCLITIIYDQSGNGNHLTQAPPGGFDGPDVDGYDNLASAIGAPVTLNGQKAYGVFMSPGTGYRNNEATGTATGDEPEGMYAVLDGTHYNDACCFDYGNAETSSTDTGAGHMEAIYLGNSTTWGYGAGDGPWIMVDMENNLFSGADEGYNSGDPSISYSFVTAAVKGGADKWAIRGGNAASGSLSTYYSGARPDYSGYNPMSKEGAIILGIGGDNSNGAQGTFYEGVMTSGYPSDDVENSVQENIVAAKYVSGSLVSGPSFTSGEVVSLRVTTPGYTTRYIAHTDTTVNTQVVDDDSSTTLKEEASWTVVTGLANSQCFSFESVDTPGSYIRHYNFELLLNANDGTKQFHEDATFCPQAPLNGEGTSLRSWSYPTRYFRHYDNVLYAASNGGVQTFDSKTSFNNDVSFEIETAFAS

SEQ ID NO: 39 - DNA, 1500 bp, Aspergillus niger
  source 1..1500: mol_type = genomic DNA
  sig_peptide 1..54
atgttctcccgccgaaacctcgtcgccctagggctggcagccaccgtcagcgccggcccctgtgacatctacgaagccggcgacacgccctgcgtagccgcgcacagcaccacccgcgccctatacagctccttcagcggcgccctctatcagctccaacgtggctccgacgacaccaccaccaccatctccccgctcacagccggtggcgtcgccgacgcctccgcccaagacaccttctgtgccaacaccacctgcctgatcaccatcatctacgaccaatccggcaacggcaaccacctgacccaagcacccccgggaggcttcgacggccccgacgtcgacggctacgacaacctcgccagcgccatcggcgcccccgtcaccctcaatggccaaaaggcctacggcgtgttcatgtcccccggcacgggctaccgtaacaacgaagccaccggcactgccaccggcgacgagcccgaaggcatgtacgccgtcctggacggcacgcactacaacgacgcctgctgcttcgactacggcaacgccgagaccagcagcaccgacactggcgccggccacatggaggccatctacctcgggaacagcaccacctggggctacggcgccggcgacgggccctggatcatggtcgatatggagaacaacctcttctccggtgctgatgagggatataactccggtgatccctcgatcagctacagctttgtcactgccgcggtcaagggcggggctgataagtgggctattcgcggtggtaatgctgcctctgggtccctctctacttactacagcggcgctcgcccggattactccggctataaccccatgagcaaggagggcgctatcatcctgggtatcggcggtgacaacagcaacggcgcccagggtaccttctacgagggtgtcatgacctccggctacccgtcggacgatgtcgagaactccgtccaggagaacatcgtggctgcgaaatacgtctccggctcgctggtcagcggcccgtcgttcacctccggagaggtggtctcgctgcgtgtcactacgcccggttacacgacgcggtatattgcgcacactgacaccactgtgaacacgcaggtcgtggacgacgatagttccaccacgctgaaggaggaggctagctggaccgtggtgaccggtctggctaatagtcagtgcttctcgttcgagtcggttgatacccctggtagctatatccggcattacaactttgagttgctgcttaatgccaatgatggcacgaagcagttccatgaggatgctactttctgtcctcaggcgccgttgaatggagagggtacttcgttgagatcgtggagttatccgacgaggtatttcagacattatgataatgtcctgtatgctgctagtaatggcggtgtgcagacgtttgattccaagacatcgtttaataatgatgttagctttgagattgagacggcgtttgcttcgtaa

SEQ ID NO: 40 - protein, 499 aa, Aspergillus niger; identical to SEQ ID NO: 38
  SIGNAL 1..18

SEQ ID NO: 41 - DNA, 1485 bp, Thermobacillus xylanilyticus
  source 1..1485: mol_type = genomic DNA
gtggcaagccgggtagtcgtaaacgccgacagggtgaagggcacgatcaaccgcaacatttacggccatttctcggaacatctcgggcgatgcatctacgaagggctgtgggtcggagaagattcgccgattccgaatacgaacgggatccgaaacgacgtgctcgaggcgctgaagcagatgaaaattccggtcctccgctggccgggcggctgcttcgcggacgaataccattggaaagacggcgtcggcccgcgggagaagcggaagcggatggtgaacacgcactggggcggcgtcatcgagaacaaccatttcggcacccatgagttcatgatgctgtgcgagctgctcggctgcgaaccgtacatcagcggcaacgtgggcagcggcaccgtgcaggagatgtccgaatgggtcgagtacattacgttcgacggcgaatcgccgatggcgaactggcgcagggagaacggacgggagaagccgtggcgcatcaagtactggggcgtcggcaacgagaactggggctgcggcggcaacatgcgcgcggaatattacgccgatctgtaccgccagttccagacgtatctgcgcaactacggcgacaacaagctgcacaagattgcgtgcggcgcgaacacggccgactaccattggacggaagtgctgatgaagcaggccgcgccgttcatgcacgggctgtcgctgcactattacaccgttccgggcccctgggagaagaaaggacccgcgaccggcttcacgacggatgaatggtgggtcacgctgaagaaggcgctgttcatggacgagctcgtcacgaagcattcggccatcatggacgtctacgatccggacaagcggatcgatctgatcgtggacgaatggggcacgtggtatgacgtcgaaccgggcacgaatccgggcttcctgtatcagcagaactcgatccgcgacgcgcttgtcgcgggcgcgacgctgcacattttccaccgccattgcgaccgcgtccggatggcgaacatcgcccagctcgtcaacgtgctgcaatccgtcatcctgacggagggcgagcggatgctgctgacgccgacctaccatgtgttcaacatgttcaaggtgcatcaggatgcggaactgctcgatacatgggagtctgtggagcgcacgggtccggaaggcgagctgccgaaggtatcggtctccgcttcccgcgcggcggacggcaagatccacatcagtctgtgcaatctggacttcgagaccggagcaagcgtggacatcgagctgcgcggcctgaacggcggcgtgagcgcaaccggcacgacgctgacgtcggaccgcatcgacgggcacaacacgttcgacgagcccgagcgggtgaaaccggccccgttccgcgacttcaaactcgagggcgggcacctgaacgcatcgctgccgccgatgtccgtgacggtgcttgagctgacggcggggtga

SEQ ID NO: 42 - protein, 494 aa, Thermobacillus xylanilyticus
MASRVVVNADRVKGTINRNIYGHFSEHLGRCIYEGLWVGEDSPIPNTNGIRNDVLEALKQMKIPVLRWPGGCFADEYHWKDGVGPREKRKRMVNTHWGGVIENNHFGTHEFMMLCELLGCEPYISGNVGSGTVQEMSEWVEYITFDGESPMANWRRENGREKPWRIKYWGVGNENWGCGGNMRAEYYADLYRQFQTYLRNYGDNKLHKIACGANTADYHWTEVLMKQAAPFMHGLSLHYYTVPGPWEKKGPATGFTTDEWWVTLKKALFMDELVTKHSAIMDVYDPDKRIDLIVDEWGTWYDVEPGTNPGFLYQQNSIRDALVAGATLHIFHRHCDRVRMANIAQLVNVLQSVILTEGERMLLTPTYHVFNMFKVHQDAELLDTWESVERTGPEGELPKVSVSASRAADGKIHISLCNLDFETGASVDIELRGLNGGVSATGTTLTSDRIDGHNTFDEPERVKPAPFRDFKLEGGHLNASLPPMSVTVLELTAG

SEQ ID NO: 43 - DNA, 1431 bp, Talaromyces cellulolyticus
  source 1..1431: mol_type = genomic DNA
atgagaatggtggtcaacaaagtcgccgtcgctgcttcagcgctatccttgggggcttatgccattgatgcccagatcagcgttgacgtgcaggcccgatatcagtctgttgatggctttggctgctcacaagcattccaacgagcggaggatatctttggcaagtacggcttgtcacccaagaatcaaagctatgttctcgacctcatgtacagcgaagagcgtggcgctggatttacgattttgcgcaacggcatcggttcgagtaactcctcaacaagcaacttgatgaattcgatcgagccgttcagccctggatcgccaagcagtacgccgaactatacctgggaccactacaacagcggccaatttcccttgtctcaacaggcaagagcacggggccttccttacatctacgctgacgcatggtctgcacctggatatatgaaaacaaaccaagacgaaaattggtcgggattcttgtgtggaattgaaggtgaaacatgcccgtctggagattggagacaagcatatgccgattacctggttcaatatgttaagttttacgccgagtccggagtcccagtgacacacttgggcttcttgaatgaacctcaggaagtggtttcatacgccagcatgggttcaaacggtacccaagccgccgagttcgtgaaaatcctcggccagactctggagcgcgaaggaattgacatcgagctcacttgctgtgatggcgttggatggagtgagcaagaagccatgatcccaggcttgcaagttgtcggtcccgatggaaaaagtgctgaagactatctctccgttgtgactggacatggctattcgtccgccccgacattccctctttcgaccaaacgacgcacctggttgactgagtggacagatctttccggagctttcactccatatacctttttcgctgacggtggcgcaggcgagggtatgacctgggcgaatcacattcaaaccgctttcgtcaacgccaacgttagtgctttcatctactggattggcgccgagaactcaaccaccaactccggaatgatcaacctgatcaacgacgaggtcatcccttccaagcgattctggtctatggcttccttcagcaagtttgtacgtcctaacgcgcaacgcgtcaaagccaccagctccgatgcatccgtgaccgtcagtgcatttgaaaacacaaacggtgtcgttgctatccaagtaatcaacaacggcacttcggctgcttccttgaccatcgatctgggaaaaacgcataaacaagtgaaaaaggttgtgccttgggtcactagcaacgattatgatttggaggaaatgagcgaaattgacgtcaagcacaactctttcctggcaagtgtacccgccaggtccttgacaagctttgttacagagtgcgagtga

SEQ ID NO: 44 - protein, 474 aa, Talaromyces cellulolyticus
MVFSKVAVAASALSLGAYAIDAQINVDLQARYQSVDGFGCSQAFQRAEDIFGKYGLSPKNQSYVLDLMYSEERGAGFTILRNGIGSSNSSTSNLMNSIEPFSPGSPSSTPNYTWDHYNSGQFPLSQQARARGLPYIYADAWSAPGYMKTNQDENWSGFLCGIEGETCPSGDWRQAYADYLVQYVKFYAESGVPVTHLGFLNEPQEVVSYASMGSNGTQAAEFVKILGQTLEREGIDIELTCCDGVGWSEQEAMIPGLQVVGPDGKSAEDYLSVVTGHGYSSAPTFPLSTKRRTWLTEWTDLSGAFTPYTFFADGGAGEGMTWANHIQTAFVNANVSAFIYWIGAENSTTNSGMINLINDEVIPSKRFWSMASFSKFVRPNAQRVKATSSDASVTVSAFENTNGVVAIQVINNGTSAASLTIDLGKTHKEVKKVVPWVTSNDYDLEEMSEIDVKHNSFLASVPARSLTSFVTECE

SEQ ID NO: 45 - DNA, 1431 bp, Talaromyces cellulolyticus; identical to SEQ ID NO: 43
  source 1..1431: mol_type = genomic DNA

SEQ ID NO: 46 - protein, 474 aa, Talaromyces cellulolyticus; identical to SEQ ID NO: 44

SEQ ID NO: 47 - DNA, 1575 bp, Talaromyces cellulolyticus
  source 1..1575: mol_type = genomic DNA
atgtggagtctgaaatcgaattccactgcactggcagttgcactgaatggtatcgtggctcttggtcagaccattaccgtcaacccaagcaccacctaccaaaccatcgatggattcggtttctctgaagccttcggatttggagcgcctatcgctagtgcatcggcttcaattcagacgcaagtgaccaactacttgtttagcactaccactggcgctggcttaaccattctgcgtaatcgcattgctgcaggctccggttcgatcgagccaaatgcgccctcaggcccaaatgctcagcctacctatacctgggatggtaatgatgccggacaggtttggtggtccaaacaagcccgcgcaaaaggggtaaaatacatctacgctgatgcttggagcgccccagcattcatgaaaacgaatgataatgtggcaaatgggggctatctctgcggcacgaccggagaaacttgttcatctggtgactggagacaagcgtatgcaaactaccttgtccagtacatcaaagactatgcaaacgagggtattactattgactttgtcggttggctgaatgagccggattattcgcccaattacgattcgatgctaatcacgagcggcacgcaagccgcctcttttatcccgactttgtataacactatcaagagcgccggactatcaactggaattgcttgctgtgatcctttcggctggagtgacgccgttacctggacagcacaacttgcatcagcgggtgccactcagtacctcgcccgtatcacctctcactggtatgcttccaagggaacctcgcccattaataccagtctcagggtctgggagactgaatacgccgacctcgacgacgcctttactacgacctggtactcaagtggagctgccaatgaaggtttaacttgggcaaaccttatctggcagggtgttgttgaggctgatctgagcgccttcttgtattggattggcgcacaatcaaacagcaatgctgctggtctcgtcacactgaacggatctacggtgcaagcatcagggactctttgggcgtttgccatgttttcgcgttttattcgacctgatgccgtgcgaattagcacttctggttcaccgagcaacgtcaatgtgggagctttcaagaacgctgacggctcgattgtggttgtcgccatcaacaacaatggcaacagtgaaaccatttctctcagtggaatcacggcctctaaagtgtctgcatactacatggacagtgcagtctcctcgccatcgactttctctgcaacattgaacggcggcacagtgggaggctcccttccagcacgctccatggtcaccttcgtgatcaccacaggctcaagtggctcagcgacaacgacaacgacaaagacgtctagcacatttaccacgtccacctcatctaccacaacgagtacctcctcgggtgccgcacaagaatggggccagtgcggtggtcagggctggacaggaccgaccacttgtgtcagtccctatgtctgcacgtactcgaacgcgtactattcgcagtgtcttcctgcttag

SEQ ID NO: 48 - protein, 455 aa, Talaromyces cellulolyticus
MWSLKSNSTALAVALNGIVALGXTITVNPSTTYQTIDGFGFSEAFGFGAPIASASASIQTQVTNYLFSTTTGAGLTILRNRIAAGSGSIEPNAPSGPNAQPTYTWDGNDAGQVWWSKQARAKGVKYIYADAWSAPAFMKTNDNVANGGYLCGTTGETCSSGDWRQAYANYLVQYIKDYANEGITIDFVGWLNEPDYSPNYDSMLITSGTQAASFIPTLYNTIKSAGLSTGIACCDPFGWSDAVTWTAQLASAGATQYLARITSHWYASKGTSPINTSLRVWETEYADLDDAFTTTWYSSGAANEGLTWANLIWQGVVEADLSAFLYWIGAQSNSNAAGLVTLNGSTVQASGTLWAFAMFSRFIRPDAVRISTSGSPSNVNVGAFKNADGSIVVVAINNNGNSETISLSGITASKVSAYYMDSAVSSPSTFSATLNGGTVGGSLPARSMVTFVITT

SEQ ID NO: 49 - DNA, 1575 bp, Talaromyces cellulolyticus; identical to SEQ ID NO: 47
  source 1..1575: mol_type = genomic DNA

SEQ ID NO: 50 - protein, 455 aa, Talaromyces cellulolyticus; identical to SEQ ID NO: 48

SEQ ID NO: 51 - DNA, 1887 bp, Ruminiclostridium papyrosolvens
  source 1..1887: mol_type = genomic DNA
atgtttaagaatatgaaaaaaacaatcagtaaagtattggtttcttcaataattatgtctgcattgttcatggtgtctgcaccagcaggagtttcggcggcaagtgatgtaacagtgaatttaggaagcacaaaacaagaaataagagggtttggtgcatctagtgcatggtgtggaacaattagcgattatgttatgaattctctctatggagatctggggtacagtattttaagattgcgtattgaagagggaataggtgatgcatggaagactggtaactttagtaaatggtcaccagagcttgctaatgctaaaaaggcaagtgcaaagggagctattgtttttgcttcaccatggaatcccccggcttcaatgcaagaaaacttttctaaaagtggtgattcaagtgcgcaacgattgagatacgataagtacaccgaatatgcgcagtatcttaacgcttatgttaaatacatgaaggataatggtgttgatttgtatgctatttctgtccaaaatgagcctgattatgcacaagattggacttggtggactccacaagaaatgcttaactttatgaaaaataatgctggatcaattaattgcagggtaatggcaccggagtcatttcaatttctgaaaaatatgtcagatccaattttaaacgatgctacggctcttgataatatggatgttcttggatgccacttttatggtacaagtgttaataatatggcgtatcctctataccaacaaaaatcagcaggaaaagagctctggatgacagagaaatattttgatgatgatactacaggaaatattatgaatatgtctaaggaaattcatgacagcatggtaactggaaacatgaatgcctatatatattggtggataacttggccaaatggtttagctacttcttcaggtacaatctacaaaagagcatatgtactcggtcaattcgcaaaattcatccgtcccggctataaaagagttgatgcaacagctactccaaatacaaatgtttatgtttcggcatatacaggcgacaataaagcggttattgttgcaataaatacaggtaccgctgcggtaagtcaaaagtttaactttcagaatggctcagcatcaagtgtagtttcatatgtaactgacagcagcagaaatatggcagctggagccaatatcgcagtaacaaatggttcctttacagctcaacttcctgctcaaagtattacaacctttgtaggaaacacagctccagttgtagtagaaccgatagatgctttcaataaaatagaagcggagaattactatgaccaatcaggaactcagactgaagcaaatagtgacggtaatggaaagaatgtaggctatatagaaaacgaagattatcttgttttcaaaaatgtagatttcggaagcggtgctgcaagttttgaggctagtgcaggcagtgcaaccaatggcggaaatatagagcttcgtctagatagtctgacaggaacattaatagggaattgcgccgttccaggtaccgggggatggcagacttggactaatgcaacctgcaatgtcagccaagtgaccggaaaacatgacgtatatctcaaatttaccggagaaagcggatacttaatgaatttagattggtttaagtttaatacaaaagtaattcctgtaggtaagttgggagatataaatggtgatgcgtcaatagattcgttagacttaatgttgataaagaagcatctgcttggtgaagcaattgaaaatacggcattggctgatttagacggaagcggtgcggtggatgcaattgatttagcacaaatgaaacagtatttactaggtataatatctgctttccccggaaaagcatag

SEQ ID NO: 52 - protein, 390 aa, Ruminiclostridium papyrosolvens
MASDVTVNLGSTKQEIRGFGASSAWCGTISDYVMNSLYGDLGYSILRLRIEEGIGDAWKTGNFSKWSPELANAKKASAKGAIVFASPWNPPASMQENFSKSGDSSAQRLRYDKYTEYAQYLNAYVKYMKDNGVDLYAISVQNEPDYAQDWTWWTPQEMLNFMKNNAGSINCRVMAPESFQFLKNMSDPILNDATALDNMDVLGCHFYGTSVNNMAYPLYQQKSAGKELWMTEKYFDDDTTGNIMNMSKEIHDSMVTGNMNAYIYWWITWPNGLATSSGTIYKRAYVLGQFAKFIRPGYKRVDATATPNTNVYVSAYTGDNKAVIVAINTGTAAVSQKFNFQNGSASSVVSYVTDSSRNMAAGANIAVTNGSFTAQLPAQSITTFVGNALE

SEQ ID NO: 53 - DNA, 912 bp, Aspergillus niger
  source 1..912: mol_type = genomic DNA
atgctatcaacccacctcctcttcctcgccaccaccctcctcacatccctcttccatcccattgccgcccatgtcgccaagcgcagtggtagcctccaacaaatcaccgatttcggtgataaccccacaggtgtaggcatgtacatctacgtgcctaataacctggcctcgaatcctggtatcgtggttgcaatccactactgcaccggcactggacccggctactacagcaactccttctacgccaccctctccgagcaatacggcttcatcgtgatctacccgtctagcccgtactccggaggctgctgggacgtgagttcgcaggcaacgctgacacataatggaggtggaaacagtaactcgattgccaacatggtcacctggacgattagcgagtacggggcggatagcaagaaggtgtttgtgacgggttcgagttcgggggctatgatgacgaacgtaatggcagcaacctaccccgaactcttcgccgctggcaccgtctactccggcgtctcagccggctgcttctactcggacactaaccaagtggacggatggaattccacctgcgcacaaggagacgtcatcaccaccccggaacactgggctagtattgccgaggcaatgtatccagggtactcgggaagccggccaaagatgcagatctaccacggcagtgtggatacgacgctgtatccgcagaattattatgagacgtgcaagcaatgggctggagtgtttgggtacgattacagtgcaccggaatcgacggaggcgaatactccgcagacgaattatgagacgacgatttggggagataatctgcaggggatctttgcgacgggcgtgggtcatacggtgccaattcatggggataaggatatggagtggtttgggtttgcttga

SEQ ID NO: 54 - protein, 303 aa, Aspergillus niger
MLSTHLLFLATTLLTSLFHPIAAHVAKRSGSLQQITDFGDNPTGVGMYIYVPNNLASNPGIVVAIHYCTGTGPGYYSNSFYATLSEQYGFIVIYPSSPYSGGCWDVSSQATLTHNGGGNSNSIANMVTWTISEYGADSKKVFVTGSSSGAMMTNVMAATYPELFAAGTVYSGVSAGCFYSDTNQVDGWNSTCAQGDVITTPEHWASIAEAMYPGYSGSRPKMQIYHGSVDTTLYPQNYYETCKQWAGVFGYDYSAPESTEANTPQTNYETTIWGDNLQGIFATGVGHTVPIHGDKDMEWFGFA

SEQ ID NO: 55 - DNA, 54 bp, synthetic construct
  source 1..54: mol_type = other DNA
atgaagctcgccactgctttcaccatccttaccgctgtcttggctgcgcccctc

SEQ ID NO: 56 - protein, 18 aa, synthetic construct
MKLATAFTILTAVLAAPL

SEQ ID NO: 57 - DNA, 72 bp, synthetic construct
  source 1..72: mol_type = other DNA
atggtcccctcaatcacacggattgtactggttaacattctgttggcgacgttggttttgggagctgcagtc

SEQ ID NO: 58 - DNA, 72 bp, synthetic construct
  source 1..72: mol_type = other DNA
atggtcccgtcgattacccgaatcgtcctcgttaacatcctcctcgccactcttgtcctgggtgcggctgtt

SEQ ID NO: 59 - DNA, 72 bp, synthetic construct
  source 1..72: mol_type = other DNA
atggtcccgtcgatcacccgtattgttttggtcaacattctccttgccaccctggtcctcggtgccgctgtt

SEQ ID NO: 60 - DNA, 72 bp, synthetic construct
  source 1..72: mol_type = other DNA
atggttccatccatcacccgaatcgtcttggtcaacattcttctcgctacccttgtcctcggtgcggccgtc

SEQ ID NO: 61 - protein, 24 aa, synthetic construct
MVPSITRIVLVNILLATLVLGAAV

SEQ ID NO: 62 - DNA, 93 bp, synthetic construct
  source 1..93: mol_type = other DNA
atgaaattcatcgttgtcgtcaccactttctcggcgattgcttcggctctcgtccttcctgagggcttcaaacctgaggccgccactgtggct

SEQ ID NO: 63 - protein, 31 aa, synthetic construct
MKFIVVVTTFSAIASALVLPEGFKPEAATVA

SEQ ID NO: 64 - DNA, 93 bp, synthetic construct
  source 1..93: mol_type = other DNA
atgttgctgatcaactttttcatcgctgttctgggagtgatatcactgtctcctattgtggttgctcgttatattcttcgacgagattgcact

SEQ ID NO: 65 - protein, 31 aa, synthetic construct
MLLINFFIAVLGVISLSPIVVARYILRRDCT

SEQ ID NO: 66 - DNA, 96 bp, synthetic construct
  source 1..96: mol_type = other DNA
atgaccatttgctgtggacggtcgaagacatgtcttttgatttttttggtgcttggttacctttgtgggcaatctcgcggccaatatgtcgacgac

SEQ ID NO: 67 - protein, 32 aa, synthetic construct
MTICCGRSKTCLLIFLVLGYLCGQSRGQYVDD

SEQ ID NO: 68 - DNA, 949 bp, synthetic construct
  source 1..949: mol_type = other DNA
tcttacggatacagtccggtcgcgacaataatatgtacatgttctctaattttgatttggaatatgcgtttttgtgactttgacaagtttagacagtcaaagtgtgtcatggattactttagaatttggaattggtatcatagtcgttggactcctataggtggcgtgacggcgtatatttggttcaacgggcttatttgttttgtttggttgttgcctttagatatggatgttgctgtatcacatggtaggcgcatgagattatatgtatcttatttatcattctattactgtattatttaatttatagtgcattataccaagtggactttccttcatgtgtcagtcacaacgaccatcaatcaaggtttttccacaatatgaagcgcgtattattcagacaacaccgtacacatgccagtccccctcgtcatttttcacgattcacattaccgtctaccgctgacgttttgcgcgggttgaagtagatctgtatgacgcgtctcttcgatcgggattgcgcggattgcgcggatgttgtcgcgtggaggtggcaaagcagctcggaatatttggagaaaatgtggctgtgcggtgtagttgtgcggctggtgcttgctggcgtggcgaagttcgcgcagcatctggccagcttatcccggcgcacaactatagttggcgcaatcttgtggctgatcaatggctggggcctaagatagtaacagaattttacagcacgaggccgcgtaagtgtcggcacgtgcggcagtgcaataagcacgctgctcaagctgcgccggccagtatcgcgttgccctctcgcccgctaaacttctgtcaccattccttcttcctcttaaagccccttgcaacctattttccgatccaatcagttccctcaatccatcccatttttgtctttctacagttaattgtataatatttcaca

SEQ ID NO: 69 - DNA, 987 bp, synthetic construct
  source 1..987: mol_type = other DNA
gtatagtatgagttaaatgtgtttaggggtaccggtgttacgggttgcagcttgttaacattattattttttcttctgctattattgttttattatcttcctagtttgaaattgtgttattatagtataggtatgatttcgtaaatagcgtgtagtcgatgggaaaggttatttgtcagtaatactcaccttatgtacttgtaagttattattgtttgcgagatatgttgtatatgccgaataaagagctatcggaaggcagagctgcgatactatgatggcaaaataaatttaggaataaggtggaattgtcagtgatactactgtaacctcagttaacagcatcgctatatttatattgccgaaggctgctggcaataaacagccaacagcgagataatattattcatctgtcagattttacgttttgaagaggttgatattgcggatagaatgtcaatgccggaattaaatgaacatgatgaggctggtcctttattattagggtgccgcagaacagccacgacacgcaaaatcataagtggagaacggccgagatcatgttactgaatatcgtacgaggcggctgcacttctgcaagaaaaaaaaccgaagtatttttgggatgcgggagatgcaagccgtttacaagtagctaattgttgtaagccgcggactgaggactaagtgctcgtgattggccggaggattccgggaagaaaagacggattttacccggcgtctaagccgggagtggcggccaatgggagcgccgagaattctgtacgaccaagcagttctcggaaaggtcggagggttagggccccgtcaggcatgccgcaacgctgctcggtaactcgagtccggggacactttatattcgatcatcttctgatcctatattttccttcactgtcttttcggtcgcttttgttgcatcttacaccctcatttctctgtcaatttacttctacatactaattcgca

SEQ ID NO: 70 - DNA, 692 bp, synthetic construct
  source 1..692: mol_type = other DNA
gatgcaaatgggtcttggtggtgaagtgagaatgttgagaggtgcggtggaagagaccattcggaaccggggatgcttgggctcgcgtgtattaggaacgatggctgctagtccccatcggtacctgctgtacgcatgcgtagcaggaagtacgcttgatggcacagtacagactgctgggtttggggtgccacaacgcatatacgtttgacgcatatgccgatttgcagcctataagacaacagaccaatacccttacctcgagaactcggttgttagcggcttttaaagttatacaagctgctctgcgcactaacgactgtatacaaattagtggaatgtgcgggtgtaggcgggaactgggcgtagaaccctctttcggcacgttgtgttgaggtttaatctcaacacattctcgaaaaactggactcatttatcgtacatctaaagacctatcatccccacacaattccctatgtcctggaataatacaactacatagccaaacgaaaacatctagtcatacaatgggcgtattcaaaaaattcgatgtcatcctacgtcggactaactttgggcattcacggcaccgttgcgaccatatgtgttattggtgctaatcatagtggcagctgatacagaaggatttggaatgattttagcggtttaggcttatgctaaacttggccagc

SEQ ID NO: 71 - DNA, 1000 bp, synthetic construct
  source 1..1000: mol_type = other DNA
acctattgctattatcgcagaacctgctattatcgtgaggtggggtcgctgagcagacaggcagtagagcaccttggatccgccctgcaatatgccctgcggctccgatagaggttccgtcggaagcggagtttccctggcaattaattgttgattccttccgtcccacgcgagcaaccttccccaccttcctttcatgatctactcgaccagcagtttcaagaccgttcagcatgcggtttagtgaatcgcgtgggtcgcacattctgcaggcggacctcagagcgtgtcggtttactgagagtccaaggatcagtttatgtaatcaagctatagatcagatatgataatcgcgggcatccatgttgcagtctagattttagatttgagtcaagagggcaaccgcgacacgacgacaacatcgcctctaagttgcgcagcaacgcactgtatgatcgctattactattattgtagtatgtagaatagatactgctttgtgtaatcgtcgaatgctgtagcttcgtgcagcgtaaagcagaatgtgaacgagacggctgacaatgggatgcgctctgattcgctcggcggagctgtcgggcgggggttagggcagatcccaggcgagtgcgggcaagggtttgctctgcaggcttgtgcatttttctcacgcgctctgattggccgcttaagagctgaacagcacaagtgtcacgagcacatggctttatttgacctgcagacctctgaaaagccacgctagtctggaaccagtttagcgcgaaagtgattcctctaccatggcgaagatgctcagccggctcaccgcgaataaataacacccgccgatccagcgccgtcacgagcaagacgaatattttgccgacgacattgaagtttcacctcttcttctcctcctcttcttctcatcctctttttcattgcgcgtctgtgtcgcttagtcgcgccaaagcatgctgctaaacgctcgtttgccctca

SEQ ID NO: 72 - DNA, 666 bp, synthetic construct
  source 1..666: mol_type = other DNA
gatatatctatttgtcacgagattattcagtaatatactataacgtattccgatagcaagtcagtggcgatggtaaatcgcagcatatcccatacgtgtcaatgtgcagcaaaaatgcattttatgcatggtctataggtaaacaagtagggtgggggtaaataaacaaggatatcaaaacgatactcgtgaccaattcactgtcagcgggatgattaatcgtattaaccgaagtgtctgtcggctcgagtggaactaaatgtctacacctatccgacagacaatcggatgactgcccgcattcatcaaagaagaaatcagaatttagcgagtaacgacaagcggtcgacagtatctaccaatggtgcatgtttgaccagagcattagctgtattagttgcaacttgatagagtaagacgcctaatacaagcacataacgccgtctgcaatggggcgccgaataatgcttggtctggggccggtctacagaatacgcgcaccgcggccgatccggaagcctgccagtgagaaacgccgctttatttgccctaaccaccacatctctgcccaaccatcgagccgccaaagtcttcactacctttccgacgagttatattcgtcgcttcgccatccctcccaaccactacttcaattcattcgaggaa

SEQ ID NO: 73 - DNA, 500 bp, synthetic construct
  source 1..500: mol_type = other DNA
gcggacgttgttccggatgaatgacttctgcgctgtgattttgtcttttgtgaacattatgtgtacgtgatgtcgagacggagggcatgtgttatgatagatagcccgttgtttgaaggaagatatgaaagcttttttatgtcaatgttatttgtctacgtttgttgtccttgatctaacgagcaatcgatatattcttaaaaatttgtattgaagtgcgaacagtggatgacagttcaacgtcctttgtggagtaggtctcggtcttacgcgtacgtcagtacccataagcgccgttcaagtaacagagtaaatttaaggaagtcaaagcaggttcaactgtatatgcgatgcagactaaatgctgctcaggtattggttgtcatagctatcattgtctcaaagttaccaatttgttactttgtgagcttgtaagtcggcttgcagtttgcacttatatattagacaacaacgtccattaaattcaactctatactcct

SEQ ID NO: 74 - DNA, 327 bp, synthetic construct
  source 1..327: mol_type = other DNA
atgtagtgatggagtcgagattagtggcgggtccgctagcctacattagctgatttgggaataagccgatggtgaatggtgtaatacagattagtattatagatagcctatgtgtggagatgcaacaatataaaattaaagaattgggttgcttaagtgactatctcgtcatcatggcggaacaatctaatcataacggtttgcatacagtaacaaaggaatgttgatacggcggcttagtacgcaatggatcgtatttgttacccggctgcgaagagtcaccagtccaaaaaatattctgtggccgtctagcatcgtccatttagt

SEQ ID NO: 75 - DNA, 514 bp, synthetic construct
  source 1..514: mol_type = other DNA
gcatatggtgttcatttggcggcaaatgaagagactcatatgcgtaacccgttacagcgaaaaccgaactaatgtgaatacaaatcgattaatagtgatgtttgaatagtggcgtagatagtgaatcgaatagagttttttgtaatttcctctgcgtatgtcaactcattttcatttattatcagtatggaatagagcgcatagtttcaaaagtaagaccctgccatcgctttttgataacatgactctacactgcaataaataataaaaccttcgaatgcatggaagaatcggatgtatgaatggaactttcgctcgaactgttggtgcctgaacctaagtttactggaattcaatcgatagaataagcaacacaaacggaacatgaacacatagacgtagtagcatttaagacaatattgtacaaatacattaaatccgatcgcaacttcagaacgcaattgtttatctaaattgtatccacaaactcccatccaaaccaaaacgtatccat

SEQ ID NO: 76 - DNA, 375 bp, synthetic construct
  source 1..375: mol_type = other DNA
atggctaagcttacctccgctgtccccgtcctcactgctcgtgacgtcgccggcgctgttgagttttggactgatcgtctcggtttttcgcgcgatttcgtcgaggacgacttcgccggtgttgtccgtgacgacgtcactctcttcatctctgccgttcaagaccaggtcgtccccgataacaccttggcctgggtttgggtccgtggcttggacgaactctatgccgagtggtctgaggttgtttctacgaacttccgcgacgcgtctggtcctgctatgactgaaatcggcgagcagccctggggtcgtgagtttgcgttgcgtgaccctgcgggtaactgtgtccacttcgtcgctgaggagcaggactag

SEQ ID NO: 77 - protein, 124 aa, synthetic construct
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 78 - DNA, 1026 bp, synthetic construct
  source 1..1026: mol_type = other DNA
atgaaaaagccagagttgactgctacttctgttgaaaagttcttgattgaaaagttcgactctgtttcggacttgatgcaattgagcgaaggcgaagaatctcgtgcattctcgttcgacgttggcgggagaggatacgttttgcgtgttaactcatgcgcagacggcttctacaaggacagatacgtttaccgtcacttcgcttcagctgctcttcctattccagaagttttggacattggcgaattctctgaatctcttacttactgcatttcacgtcgggctcaaggggtgactcttcaagaccttccagaaactgaacttcctgctgttcttcagcctgttgctgaggctatggacgctattgcagctgctgacttgtctcaaacttctgggttcgggccattcggcccacaggggattggccagtacacaacgtggagagacttcatttgcgctattgctgaccctcacgtttaccactggcagacagttatggacgacactgtgagcgcttctgtggctcaggctttggacgaattaatgttgtgggctgaagactgcccagaagttaggcaccttgttcacgctgacttcggatcaaacaacgttcttacggacaacggcagaattacggcagtgattgactggtcagaagctatgttcggcgacagccagtacgaagtggctaacattttcttctggcgtccttggttggcttgcatggaacagcagactcgttacttcgagcgtcggcacccagagttagctggctctccaagacttcgtgcttacatgcttagaattgggcttgaccaactttaccaatctttggttgacggcaacttcgacgacgctgcttgggctcagggcaggtgcgacgctattgttcgttctggcgctggcacggttggcagaactcaaattgcacgtcggtctgctgctgtttggacagacgggtgcgttgaagttttggcagactctggcaacaggagaccatcaactagaccacgtgctaaggaatag

SEQ ID NO: 79 - protein, 341 aa, synthetic construct
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 80 - DNA, 570 bp, synthetic construct
  source 1..570: mol_type = other DNA
atgactactttggatgacactgcatacagatataggacttctgtccctggtgacgctgaagctatagaggcattagatggctcattcacaacagatacagtttttcgtgtaactgctactggtgacggattcacattgagagaagtcccagtcgatcctccattgaccaaagtgttcccagatgacgaatcagacgatgaatctgatgctggtgaggatggagatccagactcaagaacattcgttgcatacggagatgatggagatttagctggatttgtcgttgtcagttacagtggttggaacaggcgtttaaccgtggaagatattgaagttgcacccgaacataggggacacggtgttggtcgtgccttaatgggcttagccaccgaatttgcaagagaaaggggagctggtcatttgtggttagaagttactaatgttaatgcccctgcaattcatgcctacaggaggatgggattcacattatgcggcttggatactgccttgtatgacggtacagcttccgacggcgagcaagctttatacatgtccatgccatgtccataa

SEQ ID NO: 81 - protein, 189 aa, synthetic construct
MTTLDDTAYRYRTSVPGDAEAIEALDGSFTTDTVFRVTATGDGFTLREVPVDPPLTKVFPDDESDDESDAGEDGDPDSRTFVAYGDDGDLAGFVVVSYSGWNRRLTVEDIEVAPEHRGHGVGRALMGLATEFARERGAGHLWLEVTNVNAPAIHAYRRMGFTLCGLDTALYDGTASDGEQALYMSMPCP

SEQ ID NO: 82 - DNA, 1233 bp, Lipomyces starkeyi
  source 1..1233: mol_type = genomic DNA
atgagtgagaaggcagagatcgaggttccgccgcaaaaatcgacattccctcgcagtgtgcacttcgctccacttcatattccactggagagacgcctacagactttggcagtcttattccacactgtcgcgctaccatactgcatcggtctgttctttctcatgctcgcgttccctcctttttggccattattggtaatgtatgtcatatacgcatacgggttcgaccactcgagctcgaacggagagatctcccgccggcgatcgccgctgtttcgaagactcccgttgttcaggctgtattgtgattacttccccatccacattcaccgggaggttccgctcgagccgacgtttcctggtcgccttcgcgaaccgagtggccttgtcgagcggtggattgcgaagatgttcggcgtgcaggacgctgttgtcgagggaaatgaatctgacgttaaggccacggccaacggcaatgggacgacgaaagaaatcggaccgacgtatgttttcggctatcatccgcatggaattgttagcttgggtgcgtttggtgctattggtacggaaggcgctggatgggagaagctctttcctgggatcccggtgtcactgctgactctcgaaacaaatttcagccttccattttacagagagtatttgctgtcacttgggattgcttcagtatctcgacggtcttgtaccaatctcctcaaacacgaccaatccatctgcatcgttatcggcggcgcccaagagtcgctcttagcggaaccaggcactctagatctgatcctcgttaaacgtcgcggttttgtcaaacttgcaatgtcaacggcgcgggtatctgaccaaccgatttgtcttgttccgatcctcagtttcggcgagaacgacgtgtacgaccaagtccgcggggaccgatcgtcgaagttgtataagatccagacttttatcaagaaagcggccgggtttacgctaccattgatgtatgcgcgcggtatatttaattacgactttgggctgatgccgtaccgcaggcaaatgacgctcgtggtcggcaagccgattgcagtgccgtacgtggcccagcctacggaggctgaaatcgaagtgtatcacaagcagtacatggatgaattgaggaggttatgggacacgtataaggacgactattttgtagaccacaagggcaagggggtcaagaattccgagatgcgttttgtggagtaa

SEQ ID NO: 83 - protein, 410 aa, Lipomyces starkeyi
MSEKAEIEVPPQKSTFPRSVHFAPLHIPLERRLQTLAVLFHTVALPYCIGLFFLMLAFPPFWPLLVMYVIYAYGFDHSSSNGEISRRRSPLFRRLPLFRLYCDYFPIHIHREVPLEPTFPGRLREPSGLVERWIAKMFGVQDAVVEGNESDVKATANGNGTTKEIGPTYVFGYHPHGIVSLGAFGAIGTEGAGWEKLFPGIPVSLLTLETNFSLPFYREYLLSLGIASVSRRSCTNLLKHDQSICIVIGGAQESLLAEPGTLDLILVKRRGFVKLAMSTARVSDQPICLVPILSFGENDVYDQVRGDRSSKLYKIQTFIKKAAGFTLPLMYARGIFNYDFGLMPYRRQMTLVVGKPIAVPYVAQPTEAEIEVYHKQYMDELRRLWDTYKDDYFVDHKGKGVKNSEMRFVE

SEQ ID NO: 84 - DNA, 1668 bp, Lipomyces starkeyi
  source 1..1668: mol_type = genomic DNA
atgtcgaccgctgcacaatctgatacagacaacgaggatatatcgactgtcgatttggttgactctcgtgcagatactcacacatcttcaaatgttatgttgcaacagcaaaaatcgcgtcggagactaatcgggaaagacgccgagccaagaacacagcatccgtctggaggcaaatcggagaaggaggagttgacgaagccggatgactcaaagggacccataaaattaagtcacatatacccgatacatgccgttagccgaggcagtattctgtcacgagagtcgacaactcctacaccgagttttgttgggtttcgaaacttagccatgatagtgctagggaagttacagtattcattattcttttggtgcgatcgggctaacattccgacagccgtcagcaatcttcgattggtgattgaaaattactcaaagtacggcgttctgatccgattcgcccgactcggtatttcacaaaaggacattctgtattgcatattcttgaccgctaccatcccgctgcacctatttattgctattgtcattgaaagactagttgcgattccgacggtaaactacgtcgcttcgctcagcgagagcgaggataaaaaacgctccaaccccaaaatgggacggaaggggggcagtatatcgattttgcgtcctaagccaaaatatatgtggcgcctgatcgtcctattgcattcaataaacgcaatggcttgcttgtgggttacgactgttgttgtttacaattctatttatcatccccttattgggacagcttgtgaatttcatgcagtgattgtgtgtcttaaggtcgcatcgtttgcgcttaccaatcgcgatcttcgggagtcgatgctgaactctcaacctgtgccagccatatacaacttggccccttatccaaaaaacttaaccctcaagaacttgtcatacttttggtgggcgccgactcttgtttatcaacctgtctatccgcgatcgccttcattccggcctttgttttttgtcaagcggattctggagatggtgggcctatcatttttaatatggttcttgtcagctcaatatgctgtgccgacgctagaaaatagtttggtgcattttcacagtttgcaattcatgggaattatggagcgactcatgaagcttgctagcattagcatggctatttggcttgctggttttttctgcatttttcagtctggactcaatgcgcttgcggaggtaatgcggtttggtgacagagccttttacgacgactggtggaacagcaaatctgtgggagagtattggcgtctgtggaataagccggttacgaattacttccggcgtcatatttacgtaccgcttgtgcgccgcgggtggaattctgcgacagccagtgtcatggtatttttcgtcagcgcggtgttgcatgagctagttgttggagttccgacgcataacgtaattggagttgcattctcgtcgatgattctacaaatcccactcatacaagtaaccgcgcctctggagaagatgcatggacctacatctggaataatagggaactgtatcttttggtttagcttcttcatcggtcagcctctgggcgtgctactttactattttgcgtggaacgttagtatgagcaaagtaaagatggtcgagagctag

SEQ ID NO: 85 - protein, 555 aa, Lipomyces starkeyi
MSTAAQSDTDNEDISTVDLVDSRADTHTSSNVMLQQQKSRRRLIGKDAEPRTQHPSGGKSEKEELTKPDDSKGPIKLSHIYPIHAVSRGSILSRESTTPTPSFVGFRNLAMIVLGKLQYSLFFWCDRANIPTAVSNLRLVIENYSKYGVLIRFARLGISQKDILYCIFLTATIPLHLFIAIVIERLVAIPTVNYVASLSESEDKKRSNPKMGRKGGSISILRPKPKYMWRLIVLLHSINAMACLWVTTVVVYNSIYHPLIGTACEFHAVIVCLKVASFALTNRDLRESMLNSQPVPAIYNLAPYPKNLTLKNLSYFWWAPTLVYQPVYPRSPSFRPLFFVKRILEMVGLSFLIWFLSAQYAVPTLENSLVHFHSLQFMGIMERLMKLASISMAIWLAGFFCIFQSGLNALAEVMRFGDRAFYDDWWNSKSVGEYWRLWNKPVTNYFRRHIYVPLVRRGWNSATASVMVFFVSAVLHELVVGVPTHNVIGVAFSSMILQIPLIQVTAPLEKMHGPTSGIIGNCIFWFSFFIGQPLGVLLYYFAWNVSMSKVKMVES

SEQ ID NO: 86 - DNA, 1719 bp, Lipomyces starkeyi
  source 1..1719: mol_type = genomic DNA
atggctcctaaatcgtcgactcgtgtgccattgtcggttaaagggcccattgattgtccatatgagggcaaagaaatgctcaacctgccgcagttcaaccgaggaacagctttcacagcagaggaacgtgacctgtttaatcttgtggggaatcttcccgctgccctgcagactctgcagaatcaagtcgacagagcgtacgatcagtattcctcgatctcgacggctttggggaagaatacctttttgatgagtttaaaggtgcaaaatgaggtcctgtattttaagttgttgcaggatcatttgaaggagatgtttagtataatttacacgccgactgagagtgaagctattgagcattattcgagactgtttagacgcccggagggctgctttctgaacatcaaccaccctgagtatatcgaacggtctctggcggcgtggggtacagaggaggacattgactatatcatcgttagtgacggcgaggagatcctcggaattggcgatcaaggagtcggagctatcggaatctcaagtgcaaaagctgtgcttatgactctatgcgccggcgtccatccatcgagatgcattccagtcgcgcttgatgttggcacggataacgagcagttgctcgaggatgagctataccttggcaataggcacaacagagtccgcggcgggcgatatgataaatttgtggacgattttgtgcaatgtgtcaagaaactgtatcctcgtgcggttctccactttgaggatttcggactacctaatgccagaagactactcgacacctacagaccacgactagcgtgctttaatgacgatgttcagggtactggcgctgtcactttggccgcactctcatcagccgtccgggtggccggaattgacttccgagacctcagaacggtgatctttggagcgggaacagcaggaactggcatcgcggatcagctgcgcgactttctcaacacacaagggatttccaagcaacaagttatcgatcatatttggcttgttgacaagccaggattgcttcttaaatcaatgcatgataaacttacatcagcgcaacgtccgtacgctgcgtcggacgatcgttggaaagagatcgacaccaagtcattgtcagaaatcgtgaagaaagtgaagccgcatgtgttgattgggtgctccacgaagccaaaagcattcaacgaagcagttcttcgtgagatggccaaacacgttgaacggccgattgtctttcccctgagcaatcccacgcgactacacgaagcgacgccggcggagatttttaagtacacggacggtaaggcgctggtggccaccggttcgccgtttgatcctgtcgatggcaaggaaattgccgagaacaataactgtttcgtgtaccctggtattggcatggggtcgatcttgagcagggccgatagagtcacagagacgatgattgcggcagtcgtgaaggagcttgcgtcgttggcgccgtcggagaaagatcctactggcgcactcttgcctgatgtggcagatattagagatatttctgcgaagatcgcgactgcagtagtgttgcaagcgttagaggagggaactgcgagagtggaagagattgaaggtatcaaagttccacgagacagagatcactgcctggaatgggtcaaagagcagatgtggaaacctgagtatagaccattgagaaaggtgtga

SEQ ID NO: 87 - protein, 572 aa, Lipomyces starkeyi
MAPKSSTRVPLSVKGPIDCPYEGKEMLNLPQFNRGTAFTAEERDLFNLVGNLPAALQTLQNQVDRAYDQYSSISTALGKNTFLMSLKVQNEVLYFKLLQDHLKEMFSIIYTPTESEAIEHYSRLFRRPEGCFLNINHPEYIERSLAAWGTEEDIDYIIVSDGEEILGIGDQGVGAIGISSAKAVLMTLCAGVHPSRCIPVALDVGTDNEQLLEDELYLGNRHNRVRGGRYDKFVDDFVQCVKKLYPRAVLHFEDFGLPNARRLLDTYRPRLACFNDDVQGTGAVTLAALSSAVRVAGIDFRDLRTVIFGAGTAGTGIADQLRDFLNTQGISKQQVIDHIWLVDKPGLLLKSMHDKLTSAQRPYAASDDRWKEIDTKSLSEIVKKVKPHVLIGCSTKPKAFNEAVLREMAKHVERPIVFPLSNPTRLHEATPAEIFKYTDGKALVATGSPFDPVDGKEIAENNNCFVYPGIGMGSILSRADRVTETMIAAVVKELASLAPSEKDPTGALLPDVADIRDISAKIATAVVLQALEEGTARVEEIEGIKVPRDRDHCLEWVKEQMWKPEYRPLRKV

SEQ ID NO: 88 - DNA, 1818 bp, Lipomyces starkeyi
  source 1..1818: mol_type = genomic DNA
atggcttcctcagttcctcaagctcccgtcacttccctactctcggaggctcgcggtaagaccgtacagcgtgataataatgaattacccggtgctcgttccattgacaccgaggctacgttccagcattggatctacgacatggttatctggagtttatcagtcgtgttcgatctatttttccgtgaaatccgcccgaggggcgcataccgaatcccgagacatggtccgattatctttgttgccgctccgcacgccaatcagtttattgaccccatcatgctcatgcgtcaaattcgaattgaagcaggtcgcagaatatcgtttctggcggccgaaagttcaatgcaccgcaaattcgtcggcactgtcgctcgatcagtttcttcaattccggttgctagagctcaggacttggcgtctcacggaactggtttgatctatattgaagataaggagaagccgttggttatcaagggccagggaaccaagtttatgaaggaatgcagtcaaggcgggttgataatgttggcgaagtcacttggcagcgccgagattgatagcatcgtgtctgatgtcgagttgattttgcgccggcctttcaaggaggagaaagccatcgagtatttattttccggtccgtccaagtttaaaaaagcgccaaaggtcgaccagtcacagatgtaccagaaggtcttcgaaagactgaatgacggtggatgtatcggtatctttcccgaaggcggttcacatgaccgaccagacttattaccactaaaagccggtgtcgcggttatggctcttggtgccctagagcagaacccagagtgcgacatcagaattgtcccttgcggtatgaactacttccaccctcacaaattccgatcacgagcagttattgagtttggtcctccacttaacgttcccaaggagcttgtcaagacgtacagtgaaggaaataagagagattctatacaccagctcttggaaatgattcatagcgcgttgttggctgttactgttacctcgccggattacgacacattgatggttattcaagcggctcgacgactatacaaacccgcacacaagaaaatcccgctatcgttggttatcgagatgaatcggcggttagtcatagggtatacacattacaaggacgatccacggattattcatcttcgggaagccgttgcgaactataacaagcagttgagacatttgggaatcctggatcatcaggttgagtacgcaacattgccaataccggagattgtcggaaaattagtctaccggtcgctgaaactatttattctggcattgggcgctctgcccggagctattcttttcgcaccggtattcatcgcgaccaagatgatttccaagaagaaggcagccgaagcgttgaaggcgtcgaccgtgaagatcgctgccagggatgttgtcgcgacatggaagattctcgtagcgctgggtctcgcgccgacgctatactggttctacgcacttttggcgacatgggcgacgtggaagtacgatcttgttcctcaagtgcgaccggtgtggctcgtgactcttgcggctatgattattttcccggcgatcacgtacgctgcacttagaattggagagatcggaatggatattttcaagtcattaaagccgcttgtgacatgcttgaacccgaataatttgaacacgattgcgaagttgcggattacgagagaggaattgtcgaaggaagtcaccgagatgattaattcattaggcccggatgtctttccagagttcgactctcaccggttaatgcagagttag

SEQ ID NO: 89 - protein, 605 aa, Lipomyces starkeyi
MASSVPQAPVTSLLSEARGKTVQRDNNELPGARSIDTEATFQHWIYDMVIWSLSVVFDLFFREIRPRGAYRIPRHGPIIFVAAPHANQFIDPIMLMRQIRIEAGRRISFLAAESSMHRKFVGTVARSVSSIPVARAQDLASHGTGLIYIEDKEKPLVIKGQGTKFMKECSQGGLIMLAKSLGSAEIDSIVSDVELILRRPFKEEKAIEYLFSGPSKFKKAPKVDQSQMYQKVFERLNDGGCIGIFPEGGSHDRPDLLPLKAGVAVMALGALEQNPECDIRIVPCGMNYFHPHKFRSRAVIEFGPPLNVPKELVKTYSEGNKRDSIHQLLEMIHSALLAVTVTSPDYDTLMVIQAARRLYKPAHKKIPLSLVIEMNRRLVIGYTHYKDDPRIIHLREAVANYNKQLRHLGILDHQVEYATLPIPEIVGKLVYRSLKLFILALGALPGAILFAPVFIATKMISKKKAAEALKASTVKIAARDVVATWKILVALGLAPTLYWFYALLATWATWKYDLVPQVRPVWLVTLAAMIIFPAITYAALRIGEIGMDIFKSLKPLVTCLNPNNLNTIAKLRITREELSKEVTEMINSLGPDVFPEFDSHRLMQS

SEQ ID NO: 90 - DNA, 1602 bp, Lipomyces starkeyi
  source 1..1602: mol_type = genomic DNA
atgccaagggtcttcatcggtgcactggatgccggcaccacgtctacaagattcattatatttgacgatgctggtaggccttacgctacacaccagattgaattcgagcagcattatccacatgctggctggcacgagcaatacccttacgagattatcaagtgcgcgaatgactgtattgagggcgctgtcaagaagtttgtcgcagatggatacaatgtctccgacataaaggcggttggaattacaaaccagcgtgaatccactgtcgtctgggattctgagacggggaagccactgtataatgcaattgcttggcctgacacgcgtactgcgcgactagtacatcacttcaagaacaagaacggcgccgaagacctagtgcgaatctgcggtctgccactgtctacctatccgtccgcgctcaagctggtgtggctgttagaaaacgttcacggcgtccaggaggcacgaaagcgtggaactttgatgttcggtaccgttgacacctggttagtgtacaatctgaccggtggcggtaagactggaaccgacaccaggtttgtgacagataccaccaatgcctcgcgaactatgttcctaaatattaactctctcaagtacgatgatttcctccttgatttctttggagtttctcaaggagtgagactccctgacgttgtgtgctccgcagacgacaaagcttacggtaatatcgcttccggtgtcctcgctggtgtaccaattgcgtcgtgtctcggtgaccagtcagccgcgttggtcggacagcgggcgttcagtgtgggcatgggcaagaacacgtacggaactggtttgttcttgctgtacaacactggtgaggagccagtcttctcgaagaacggtctcttaaccactgtcggatatcatttcaagggaaagaagcctatatacgctctggaagggtccattgctgtcggtggcgctgcagttaagttcttgcgagataatttacggttgatttcgttctccgatgaggtcgggcaattggcagcgaaggtccccgacgctggtggggtcatttttgtcacggccttctcgggactgttcgcgccttactggattgacgatacccagggaaccatctacggtatcacgaactacacaaccaaagaacacattgctcgagcaaccattgaggccacctgttatcagactagagcagttttggaagctatggctaaggattctggctacgagttgaagacgctcaaggtcgacggtggcatgagtaactcggatatatgcatgcaaatccaatccgacattattggcattgacgtcgttcggccggagtatagggagactaccgcattgggggcagccattgcagcaggttttgcagttggagtctatagtagctttgaggagttgaagcgagtgaacacggatggagagaccacattcaagccttccatcactgagaagaagcgcgagaagttgtataacctctggcagcgtgctgtatcgcgatgtggtggttggttgcgggatgaggatgacgactctgagattgaggaggagacaagagttggggatgggacccaaaagcagtttgcatag

SEQ ID NO: 91 - protein, 533 aa, Lipomyces starkeyi
MPRVFIGALDAGTTSTRFIIFDDAGRPYATHQIEFEQHYPHAGWHEQYPYEIIKCANDCIEGAVKKFVADGYNVSDIKAVGITNQRESTVVWDSETGKPLYNAIAWPDTRTARLVHHFKNKNGAEDLVRICGLPLSTYPSALKLVWLLENVHGVQEARKRGTLMFGTVDTWLVYNLTGGGKTGTDTRFVTDTTNASRTMFLNINSLKYDDFLLDFFGVSQGVRLPDVVCSADDKAYGNIASGVLAGVPIASCLGDQSAALVGQRAFSVGMGKNTYGTGLFLLYNTGEEPVFSKNGLLTTVGYHFKGKKPIYALEGSIAVGGAAVKFLRDNLRLISFSDEVGQLAAKVPDAGGVIFVTAFSGLFAPYWIDDTQGTIYGITNYTTKEHIARATIEATCYQTRAVLEAMAKDSGYELKTLKVDGGMSNSDICMQIQSDIIGIDVVRPEYRETTALGAAIAAGFAVGVYSSFEELKRVNTDGETTFKPSITEKKREKLYNLWQRAVSRCGGWLRDEDDDSEIEEETRVGDGTQKQFA

SEQ ID NO: 92 - DNA, 1896 bp, Lipomyces starkeyi
  source 1..1896: mol_type = genomic DNA
atgtcatttcgccaggcattgttctcagctacccggactcatcgtgtgctgcttcgctccgttgccgcgggcgggatcctgagcaccacagcgttcttcctctcgaacacagactcgttcggctcaatacaccaccaggctgagcaggacgtgccattgaactatcctgtcccggccccctttgccttgcccccgtcccgagaagaacagatcaagaagttggaaaccgaacagttcgacttgctgatcataggcggtggagctaccggtgccggctgcgcattggatgccgtcagcagaggattaaaggtcgcgttggttgagcgtgatgactttgcatgtggtacaagtagtaggagtacgaagcttgtacacggtggtgtgagatacttggagaaagcattctggaacctcgactatgagcagtacaaattggtcaaggaggcacttgctgagcgtgctactttcttgaaaattgcgcctcatttgagtttcccgctcccaatcatggtccctgtctacaagtggtggcaagtcccatattactgggctggaaccaagatgtacgatcttattgctggaaaggagaacatggaatcaagttacttcatgggccgtggtaagaccctcgagaacttcccaatgcttaagcctcagaacttgaagggtgccattgtctactacgacggtagtcacaatgactcgcgaatgaacactgctatcgctctcactgctgctcagaaaggtgccgttatcctgaaccacatggaggttacagagttgtccaaggacgcgtctggtcgcgtcaatggtgctgttgtccgcgacaatgacggtactgctgggaagatccaggttgatgctaagggtgttattaacgcgaccggtccgttcgcggaccggatccgtcagctcgatactcctgttgcgatcgacatcgtcgctccatcatccggtgtgcatgtgattcttcctgactactactgctctcccagcatgggtttgatcgacccagctacttctgatggacgagtagttttctttcttccatggcaaggccatactcttgccggtacaaccgactctcctaccacagtcacgaaggatcctattccttccgaggacgagataagctggatcttgaacgaaattcaacactacgttgccgatgatattaccgttcgccgggaagatgttcttgccgcttggagcggtatccgtccattagtccgcgacccacgtgctaagaacacagagtcgctggtccgcaaccatttgatcaccctttccgacagtggtctcttgactgtcgctggcggcaagtggactacctatcgagaaatggcagaagacactgtcaacacctctgtcaaagagttcggtcttgagccgaccgccccatgtggaaccaaggatatcaagcttgtcggggctgagggatacagaaaactcatgttcattcacttgatccagacatttggcattgagactggcatagccaagcatcttgctgacaactatggagatcgtgcgtacgacgtcgtcaggctttcagccactacgggcgagagatggcccacaagaggtgtcaagctgtcgccagcttatccgtacattgatggcgagatccgttacgcagttcagcacgagtatgctcgaacggctgtcgacgtgctgtcgcgacgcatccgtctcgcgttcttgaactccaaggctgcgttggagagcttgcccaaggtcatcgatatcatggccgaagagttgaagtgggacaaggcgcgtcaggacaaggagtgggatgacaccatcaggttcttgtacagcatgggtctccagaacgggaagtacttctcgagagcggacgttgagagcggaaaaacaaaaatgttgggttaa

SEQ ID NO: 93 - protein, 631 aa, Lipomyces starkeyi
MSFRQALFSATRTHRVLLRSVAAGGILSTTAFFLSNTDSFGSIHHQAEQDVPLNYPVPAPFALPPSREEQIKKLETEQFDLLIIGGGATGAGCALDAVSRGLKVALVERDDFACGTSSRSTKLVHGGVRYLEKAFWNLDYEQYKLVKEALAERATFLKIAPHLSFPLPIMVPVYKWWQVPYYWAGTKMYDLIAGKENMESSYFMGRGKTLENFPMLKPQNLKGAIVYYDGSHNDSRMNTAIALTAAQKGAVILNHMEVTELSKDASGRVNGAVVRDNDGTAGKIQVDAKGVINATGPFADRIRQLDTPVAIDIVAPSSGVHVILPDYYCSPSMGLIDPATSDGRVVFFLPWQGHTLAGTTDSPTTVTKDPIPSEDEISWILNEIQHYVADDITVRREDVLAAWSGIRPLVRDPRAKNTESLVRNHLITLSDSGLLTVAGGKWTTYREMAEDTVNTSVKEFGLEPTAPCGTKDIKLVGAEGYRKLMFIHLIQTFGIETGIAKHLADNYGDRAYDVVRLSATTGERWPTRGVKLSPAYPYIDGEIRYAVQHEYARTAVDVLSRRIRLAFLNSKAALESLPKVIDIMAEELKWDKARQDKEWDDTIRFLYSMGLQNGKYFSRADVESGKTKMLG

SEQ ID NO: 94 - DNA, 1068 bp, Lipomyces starkeyi
  source 1..1068: mol_type = genomic DNA
atgcttaacctcaaagtatctgttatcggttctggcaactggggcagcgccattgcgaaaatagtggccgagaacgcggctgagaacagcgacattttcgagaccgacgttcgcatgtgggttttcgaggagcttgttgctggccagaagctcaccgaaattatcaatacccagcacgagaacgtcaagtacttgccaggcatcaaactgccgtccaacgtcgtggctgtgccagaccttttggacgccgtcaaggatgccaacctcttagtcttcaacgtgcctcatcagtttcttccacggatatgtagccagctgaaaggcaaagtgccgtccaccgtcagagctgtgtcttgcatcaaaggtgtcgaggtttctggcgatggcatcaccatcatcgcagactatatctctcaggaactcggcatttactgcggtgccttgtctggcgccaatcttgctcccgaagttgcgcaagagaaattttccgagacgacaatcgcgtacaaagttcccgatccaagcgactccattgacgcacgcgtcgtaaggacgctcttccacagaccttacttccacgtaaacgtcgttggtgatgtcgccggtgtgtctctttgtggtgctttaaaaaacattgtggcattggcatcaggatttgtagacggaatggcatggggcgataatgccaaggctgccattattcgaagaggattgttagagatgaccaagtttggccgtgaattcttcccggaatgcaatgccagtaccttcaccgaagaatcatgtggtgttgccgatgtcattacttcctgctctggtgggcgcaataacaagcttggtgcggcgatgattaagaccaggcgacccatttttgagcttgaggaagagttgctcaagggtcagaaggctcaaggagttacgactgcccaggaggtgcacgaatttttggagcggcggggcaagttggaagacttccctcttttcaccgcagtccacgacattattttcaacggactcgacagctatgctctgccgcagattctcgaagatgtcgagcagaagttctaa

SEQ ID NO: 95 - protein, 355 aa, Lipomyces starkeyi
MLNLKVSVIGSGNWGSAIAKIVAENAAENSDIFETDVRMWVFEELVAGQKLTEIINTQHENVKYLPGIKLPSNVVAVPDLLDAVKDANLLVFNVPHQFLPRICSQLKGKVPSTVRAVSCIKGVEVSGDGITIIADYISQELGIYCGALSGANLAPEVAQEKFSETTIAYKVPDPSDSIDARVVRTLFHRPYFHVNVVGDVAGVSLCGALKNIVALASGFVDGMAWGDNAKAAIIRRGLLEMTKFGREFFPECNASTFTEESCGVADVITSCSGGRNNKLGAAMIKTRRPIFELEEELLKGQKAQGVTTAQEVHEFLERRGKLEDFPLFTAVHDIIFNGLDSYALPQILEDVEQKF

SEQ ID NO: 96 - DNA, 999 bp, synthetic construct
  source 1..999: mol_type = other DNA
  sig_peptide 1..78
atgaagttcctccaggccaagggttccttgctctcgtctggcatttacctcattgccctcgcccccttcgtcaacgccaagtgcgctcttccttcgacctattcctggacttcgaccgatgctctcgccacccccaagtctggttggaccgccctcaaggacttcaccgatgtcgtctctaacggcaagcacattgtctatgcttccactaccgacacccagggtaattacggctccatgggcttcggcgccttctcggactggtcggacatggcctccgcttcccagactgccacctccttctccgctgtcgctcccaccttgttctacttccagcctaagtccatctgggttctcgcctaccagtggggctcctccactttcacctaccgcacctcccaggatcccaccaatgtcaacggctggtcctccgagcaggctcttttcactggcaagatctccggctcctccaccggtgccattgatcagactgtcattggtgatgatactaatatgtatcttttcttcgccggcgacaatggcaagatctaccgttcctccatgtctatcaatgacttccccggttccttcggctcccagtacgaggagatcctctccggcgctaccaacgatttgttcgaggctgtccaggtctacaccgtcgacggcggcgagggtgactccaagtacctcatgatcgtcgaggctatcggttccaccggtcaccgttatttccgctccttcactgcctcctccctcggcggtgagtggaccgcccaggctgcctccgaggatcagcccttcgctggcaaggccaactccggcgccacctggaccgacgacatctcccacggtgacttggtccgcaacaaccctgatcagaccatgactgtcgatccttgcaacctccagcttctctaccagggccacgaccccaactccaattccgagtacaacctcttgccctggaagcccggtgttctcaccttgaagcagtaa

SEQ ID NO: 97 - protein, 325 aa, synthetic construct
  SIGNAL 1..19
MKIRTSLLTAVVLVNAVLAKCALPSTYSWTSTDALATPKSGWTALKDFTDVVSNGKHIVYASTTDTQGNYGSMGFGAFSDWSDMASASQTATSFSAVAPTLFYFQPKSIWVLAYQWGSSTFTYRTSQDPTNVNGWSSEQALFTGKISGSSTGAIDQTVIGDDTNMYLFFAGDNGKIYRSSMSINDFPGSFGSQYEEILSGATNDLFEAVQVYTVDGGEGDSKYLMIVEAIGSTGHRYFRSFTASSLGGEWTAQAASEDQPFAGKANSGATWTDDISHGDLVRNNPDQTMTVDPCNLQLLYQGHDPNSNSEYNLLPWKPGVLTLKQ

SEQ ID NO: 98 - DNA, 978 bp, synthetic construct
  source 1..978: mol_type = other DNA
  sig_peptide 1..57
atgaagatacggacttctcttcttacggccgtggtattggtcaatgccgtcctcgccaagtgcgctcttccttcgacctattcctggacttcgaccgatgctctcgccacccccaagtctggttggaccgccctcaaggacttcaccgatgtcgtctctaacggcaagcacattgtctatgcttccactaccgacacccagggtaattacggctccatgggcttcggcgccttctcggactggtcggacatggcctccgcttcccagactgccacctccttctccgctgtcgctcccaccttgttctacttccagcctaagtccatctgggttctcgcctaccagtggggctcctccactttcacctaccgcacctcccaggatcccaccaatgtcaacggctggtcctccgagcaggctcttttcactggcaagatctccggctcctccaccggtgccattgatcagactgtcattggtgatgatactaatatgtatcttttcttcgccggcgacaatggcaagatctaccgttcctccatgtctatcaatgacttccccggttccttcggctcccagtacgaggagatcctctccggcgctaccaacgatttgttcgaggctgtccaggtctacaccgtcgacggcggcgagggtgactccaagtacctcatgatcgtcgaggctatcggttccaccggtcaccgttatttccgctccttcactgcctcctccctcggcggtgagtggaccgcccaggctgcctccgaggatcagcccttcgctggcaaggccaactccggcgccacctggaccgacgacatctcccacggtgacttggtccgcaacaaccctgatcagaccatgactgtcgatccttgcaacctccagcttctctaccagggccacgaccccaactccaattccgagtacaacctcttgccctggaagcccggtgttctcaccttgaagcagtaa

SEQ ID NO: 99 - DNA, 1500 bp, synthetic construct
  source 1..1500: mol_type = other DNA
  sig_peptide 1..54
atgttctcccgccgtaacctcgtcgccctcggtctcgccgccaccgtctccgccggcccctgcgacatctacgaggccggcgacactccctgcgtcgccgctcactccaccacccgcgccctctactcctccttctccggcgccctctaccagctccagcgtggctccgacgacaccaccaccaccatctcccctctcaccgccggcggcgtcgccgacgcctccgcccaggacaccttctgcgccaacaccacctgcttgatcaccatcatctacgaccagtccggcaacggcaaccacctcacccaggccccccctggtggcttcgacggccccgacgtcgacggctacgacaacctcgcctccgccatcggcgcccccgtcaccctcaatggccagaaggcctacggcgtcttcatgtcccccggcactggctaccgtaacaacgaggccaccggcaccgccaccggcgacgagcccgagggcatgtacgccgtcctcgacggcactcactacaacgacgcctgctgcttcgactacggcaacgccgagacctcctccaccgacactggcgccggccacatggaggccatctacctcggtaactccaccacctggggctacggcgccggtgacggtccctggatcatggtcgatatggagaacaacctcttctccggtgctgatgagggttataactccggtgatccctcgatctcctactccttcgtcactgccgctgtcaagggcggtgctgataagtgggctattcgcggtggtaatgctgcctctggttccctctctacttactactccggcgctcgccctgattactccggctataaccccatgtccaaggagggcgctatcatcctcggtatcggcggtgacaactccaacggcgcccagggtaccttctacgagggtgtcatgacctccggctacccttcggacgatgtcgagaactccgtccaggagaacatcgtcgctgctaagtacgtctccggctcgctcgtctccggcccttcgttcacctccggtgaggtcgtctcgctccgtgtcactactcccggttacactactcgttatattgctcacactgacaccactgtcaacactcaggtcgtcgacgacgattcctccaccactctcaaggaggaggcttcctggaccgtcgtcaccggtctcgctaattcccagtgcttctcgttcgagtcggttgatacccctggttcctatatccgtcactacaacttcgagttgctccttaatgccaatgatggcactaagcagttccacgaggatgctactttctgccctcaggctcctttgaatggtgagggtacttcgttgcgttcgtggtcctatcctactcgttatttccgtcactatgataatgtcctctatgctgcttccaatggcggtgtccagactttcgattccaagacctcgttcaataatgatgtttccttcgagattgagactgctttcgcttcgtaa

SEQ ID NO: 100 - protein, 500 aa, synthetic construct
  SIGNAL 1..19
MKIRTSLLTAVVLVNAVLAGPCDIYEAGDTPCVAAHSTTRALYSSFSGALYQLQRGSDDTTTTISPLTAGGVADASAQDTFCANTTCLITIIYDQSGNGNHLTQAPPGGFDGPDVDGYDNLASAIGAPVTLNGQKAYGVFMSPGTGYRNNEATGTATGDEPEGMYAVLDGTHYNDACCFDYGNAETSSTDTGAGHMEAIYLGNSTTWGYGAGDGPWIMVDMENNLFSGADEGYNSGDPSISYSFVTAAVKGGADKWAIRGGNAASGSLSTYYSGARPDYSGYNPMSKEGAIILGIGGDNSNGAQGTFYEGVMTSGYPSDDVENSVQENIVAAKYVSGSLVSGPSFTSGEVVSLRVTTPGYTTRYIAHTDTTVNTQVVDDDSSTTLKEEASWTVVTGLANSQCFSFESVDTPGSYIRHYNFELLLNANDGTKQFHEDATFCPQAPLNGEGTSLRSWSYPTRYFRHYDNVLYAASNGGVQTFDSKTSFNNDVSFEIETAFAS

SEQ ID NO: 101 - DNA, 1503 bp, synthetic construct
  source 1..1503: mol_type = other DNA
  sig_peptide 1..57
atgaagatacggacttctcttcttacggccgtggtattggtcaatgccgtcctcgccggcccctgcgacatctacgaggccggcgacactccctgcgtcgccgctcactccaccacccgcgccctctactcctccttctccggcgccctctaccagctccagcgtggctccgacgacaccaccaccaccatctcccctctcaccgccggcggcgtcgccgacgcctccgcccaggacaccttctgcgccaacaccacctgcttgatcaccatcatctacgaccagtccggcaacggcaaccacctcacccaggccccccctggtggcttcgacggccccgacgtcgacggctacgacaacctcgcctccgccatcggcgcccccgtcaccctcaatggccagaaggcctacggcgtcttcatgtcccccggcactggctaccgtaacaacgaggccaccggcaccgccaccggcgacgagcccgagggcatgtacgccgtcctcgacggcactcactacaacgacgcctgctgcttcgactacggcaacgccgagacctcctccaccgacactggcgccggccacatggaggccatctacctcggtaactccaccacctggggctacggcgccggtgacggtccctggatcatggtcgatatggagaacaacctcttctccggtgctgatgagggttataactccggtgatccctcgatctcctactccttcgtcactgccgctgtcaagggcggtgctgataagtgggctattcgcggtggtaatgctgcctctggttccctctctacttactactccggcgctcgccctgattactccggctataaccccatgtccaaggagggcgctatcatcctcggtatcggcggtgacaactccaacggcgcccagggtaccttctacgagggtgtcatgacctccggctacccttcggacgatgtcgagaactccgtccaggagaacatcgtcgctgctaagtacgtctccggctcgctcgtctccggcccttcgttcacctccggtgaggtcgtctcgctccgtgtcactactcccggttacactactcgttatattgctcacactgacaccactgtcaacactcaggtcgtcgacgacgattcctccaccactctcaaggaggaggcttcctggaccgtcgtcaccggtctcgctaattcccagtgcttctcgttcgagtcggttgatacccctggttcctatatccgtcactacaacttcgagttgctccttaatgccaatgatggcactaagcagttccacgaggatgctactttctgccctcaggctcctttgaatggtgagggtacttcgttgcgttcgtggtcctatcctactcgttatttccgtcactatgataatgtcctctatgctgcttccaatggcggtgtccagactttcgattccaagacctcgttcaataatgatgtttccttcgagattgagactgctttcgcttcgtaa

SEQ ID NO: 102 - DNA, 1503 bp, synthetic construct; identical to SEQ ID NO: 101
  source 1..1503: mol_type = other DNA
  sig_peptide 1..57

SEQ ID NO: 103 - protein, 500 aa, synthetic construct; identical to SEQ ID NO: 100
  SIGNAL 1..19

SEQ ID NO: 104 - DNA, 57 bp, Lipomyces starkeyi
  source 1..57: mol_type = genomic DNA
atgaagatacggacttctcttcttacggccgtggtattggtcaatgccgtcctcgcc

SEQ ID NO: 105 - protein, 19 aa, Lipomyces starkeyi
MKIRTSLLTAVVLVNAVLA

SEQ ID NO: 106 - DNA, 78 bp, Aspergillus niger
  source 1..78: mol_type = genomic DNA
atgaagttcctccaggccaagggttccttgctctcgtctggcatttacctcattgccctcgcccccttcgtcaacgcc

SEQ ID NO: 107 - protein, 26 aa, Aspergillus niger
MKFLQAKGSLLSSGIYLIALAPFVNA

SEQ ID NO: 108 - DNA, 54 bp, Aspergillus phoenicis
  source 1..54: mol_type = genomic DNA
atgttctcccgccgtaacctcgtcgccctcggtctcgccgccaccgtctccgcc

SEQ ID NO: 109 - protein, 18 aa, Aspergillus phoenicis
MFSRRNLVALGLAATVSA

SEQ ID NO: 110 - DNA, 495 bp, synthetic construct
  source 1..495: mol_type = other DNA
gcaaattgatgtggtcaagttgtgagtgtgcggaggcgaaaggttgagaaatgaatacgaacaagtgggagtggaaaaggataggcgacgtgggcggggacagctttataggaaaatgatgcgcgagtgatgggctgaaggtaaaccctagcccggagctggcgggaatgcggttgggtggaattgtggttttagcgtgggacgcggcggcatgcgcaaaataaaaaatgtggtgtgcaaatcgtggaaattgcggtctgttttttgggagttttgcaagtgtgcgctgttgtgttgttcagtatgtgggccgtcgtgcgagtctgcggcgttcgttctgcaccgtgattttgtgtttccaccgtttgatgaaattcccctagccctcatcagtcgcgcccttttatatccgtgctcccccctccggctagtttatttcttaacctgtttaatcttccatctcaactctactttcactcattatcttcaagcaac

SEQ ID NO: 111 - DNA, 1431 bp, synthetic construct
  source 1..1431: mol_type = other DNA
atgcgtatggtcgtcaacaaggtcgccgtcgctgcttccgctctctccttgggtgcttatgccattgatgcccagatctccgttgacgtccaggcccgttatcagtctgttgatggcttcggctgctcccaggccttccagcgtgctgaggatatcttcggcaagtacggcttgtcccccaagaatcagtcctatgttctcgacctcatgtactccgaggagcgtggcgctggtttcactattttgcgcaacggcatcggttcgtccaactcctccacctccaacttgatgaattcgatcgagcctttctcccctggttcgccctcctccactcctaactatacctgggaccactacaactccggccagttccccttgtctcagcaggcccgtgcccgtggccttccttacatctacgctgacgcctggtctgcccctggttatatgaagaccaaccaggacgagaattggtcgggtttcttgtgcggtattgagggtgagacctgcccttctggtgattggcgtcaggcctatgccgattacctcgttcagtatgttaagttctacgccgagtccggtgtccccgtcacccacttgggcttcttgaatgagcctcaggaggtcgtttcctacgcctccatgggttccaacggtacccaggccgccgagttcgtcaagatcctcggccagactctcgagcgcgagggtattgacatcgagctcacttgctgcgatggcgttggttggtccgagcaggaggccatgatccccggcttgcaggttgtcggtcccgatggtaagtccgctgaggactatctctccgttgtcactggtcacggctattcgtccgcccctaccttccctctttcgaccaagcgtcgcacctggttgactgagtggaccgatctttccggtgctttcactccctataccttcttcgctgacggtggcgccggcgagggtatgacctgggctaatcacattcagaccgctttcgtcaacgccaacgtttccgctttcatctactggattggcgccgagaactccaccaccaactccggtatgatcaacctcatcaacgacgaggtcatcccttccaagcgtttctggtctatggcttccttctccaagttcgtccgtcctaacgctcagcgcgtcaaggccacctcctccgatgcctccgtcaccgtctccgccttcgagaacaccaacggtgtcgttgctatccaggtcatcaacaacggcacttcggctgcttccttgaccatcgatctcggtaagactcacaagcaggtcaagaaggttgtcccttgggtcacttccaacgattatgatttggaggagatgtccgagattgacgtcaagcacaactctttcctcgcctccgtccccgcccgttccttgacctccttcgttaccgagtgcgagtaa

SEQ ID NO: 112 - DNA, 1575 bp, synthetic construct
  source 1..1575: mol_type = other DNA
atgtggtccctcaagtcgaattccactgccctcgccgttgccctcaatggtatcgtcgctcttggtcagaccattaccgtcaacccctccaccacctaccagaccatcgatggtttcggtttctctgaggccttcggtttcggtgctcctatcgcttccgcctcggcttccattcagactcaggtcaccaactacttgttctccactaccactggcgctggcctcaccattctccgtaatcgcattgctgccggctccggttcgatcgagcccaatgctccctccggccccaatgctcagcctacctatacctgggatggtaatgatgccggtcaggtttggtggtccaagcaggcccgcgccaagggtgtcaagtacatctacgctgatgcttggtccgcccccgccttcatgaagactaatgataatgtcgccaatggtggctatctctgcggcactaccggtgagacttgctcctctggtgactggcgtcaggcttatgccaactaccttgtccagtacatcaaggactatgccaacgagggtattactattgacttcgtcggttggctcaatgagcctgattattcgcccaattacgattcgatgctcatcacttccggcactcaggccgcctctttcatccctactttgtataacactatcaagtccgccggtctctccactggtattgcttgctgcgatcctttcggctggtccgacgccgttacctggaccgcccagcttgcctccgctggtgccactcagtacctcgcccgtatcacctctcactggtatgcttccaagggtacctcgcccattaatacctccctccgtgtctgggagactgagtacgccgacctcgacgacgccttcactactacctggtactcctccggtgctgccaatgagggtctcacttgggccaaccttatctggcagggtgttgttgaggctgatctctccgccttcttgtattggattggcgcccagtccaactccaatgctgctggtctcgtcaccctcaacggttctactgtccaggcctccggtactctttgggctttcgccatgttctcgcgtttcattcgtcctgatgccgtccgtatttccacttctggttccccttccaacgtcaatgtcggtgctttcaagaacgctgacggctcgattgtcgttgtcgccatcaacaacaatggcaactccgagaccatttctctctccggtatcactgcctctaaggtctctgcctactacatggactccgccgtctcctcgccctcgactttctctgccaccttgaacggcggcaccgtcggtggctcccttcccgcccgctccatggtcaccttcgtcatcaccaccggctcctccggctccgctaccactaccactaccaagacttcttccaccttcaccacttccacctcctctaccaccacttccacctcctcgggtgccgcccaggagtggggccagtgcggtggtcagggctggaccggtcctaccacttgcgtctccccctatgtctgcacttactcgaacgcttactattcgcagtgccttcctgcttaa

SEQ ID NO: 113 - DNA, 1887 bp, synthetic construct
  source 1..1887: mol_type = other DNA
atgttcaagaatatgaagaagaccatctccaaggtcttggtttcttccatcattatgtctgccttgttcatggtctctgcccccgccggtgtttcggctgcctccgatgtcaccgtcaatctcggttccaccaagcaggagatccgtggtttcggtgcctcttccgcctggtgcggtaccatttccgattatgttatgaattctctctatggtgatctcggttactccattctccgtttgcgtattgaggagggtatcggtgatgcctggaagactggtaacttctccaagtggtcccccgagcttgctaatgctaagaaggcctccgccaagggtgctattgttttcgcttccccctggaatccccctgcttccatgcaggagaacttctctaagtccggtgattcctccgctcagcgtttgcgttacgataagtacaccgagtatgctcagtatcttaacgcttatgttaagtacatgaaggataatggtgttgatttgtatgctatttctgtccagaatgagcctgattatgcccaggattggacttggtggactccccaggagatgcttaacttcatgaagaataatgctggttccattaattgccgtgtcatggcccctgagtccttccagttcctcaagaatatgtccgatcccattctcaacgatgctactgctcttgataatatggatgttcttggttgccacttctatggtacctccgttaataatatggcttatcctctctaccagcagaagtccgccggtaaggagctctggatgaccgagaagtatttcgatgatgatactaccggtaatattatgaatatgtctaaggagattcacgactccatggtcactggtaacatgaatgcctatatctattggtggatcacttggcccaatggtctcgctacttcttccggtaccatctacaagcgtgcctatgtcctcggtcagttcgccaagttcatccgtcccggctataagcgtgttgatgccaccgctactcccaataccaatgtttatgtttcggcctataccggcgacaataaggctgttattgttgccatcaataccggtaccgctgctgtctcccagaagttcaacttccagaatggctccgcctcctccgtcgtttcctatgtcactgactcctcccgtaatatggccgctggtgccaatatcgccgtcaccaatggttccttcaccgctcagcttcctgctcagtccattaccaccttcgtcggtaacaccgctcccgttgtcgtcgagcctatcgatgctttcaataagatcgaggctgagaattactatgaccagtccggtactcagactgaggccaattccgacggtaatggtaagaatgtcggctatatcgagaacgaggattatcttgttttcaagaatgtcgatttcggttccggtgctgcctccttcgaggcttccgccggctccgccaccaatggcggtaatatcgagcttcgtctcgattccctcaccggtaccctcatcggtaattgcgccgttcccggtaccggtggttggcagacttggactaatgccacctgcaatgtctcccaggtcaccggtaagcacgacgtctatctcaagttcaccggtgagtccggttacctcatgaatctcgattggttcaagttcaataccaaggtcattcctgtcggtaagttgggtgatatcaatggtgatgcttccatcgattcgctcgacctcatgttgatcaagaagcacctccttggtgaggccattgagaatactgccttggctgatctcgacggttccggtgctgtcgatgccattgatctcgcccagatgaagcagtatctcctcggtatcatctctgctttccccggtaaggcctaa

SEQ ID NO: 114 - DNA, 57 bp, Lipomyces starkeyi
  source 1..57: mol_type = genomic DNA
atgaagtcgagcattatcctatcatccttcctttcggcgctggctgtcactgccgac

SEQ ID NO: 115 - protein, 19 aa, Lipomyces starkeyi
MKSSIILSSFLSALAVTAD

SEQ ID NO: 116 - DNA, 84 bp, Lipomyces starkeyi
  source 1..84: mol_type = other DNA
atgacgacaaaaacagcagtgatgtctttctgccgcctccttataggactggctttagtgctgccatcgattgtgaacggctac

SEQ ID NO: 117 - protein, 28 aa, Lipomyces starkeyi
MTTKTAVMSFCRLLIGLALVLPSIVNGY

SEQ ID NO: 118 - DNA, 72 bp, Lipomyces starkeyi
  source 1..72: mol_type = genomic DNA
atgcgatgccgaccgccaattatttccggcctattgacgctggtagcattatctacattagtctcatgcctc

SEQ ID NO: 119 - protein, 24 aa, Lipomyces starkeyi
MRCRPPIISGLLTLVALSTLVSCL

SEQ ID NO: 120 - DNA, 84 bp, Lipomyces starkeyi
  source 1..84: mol_type = genomic DNA
atgaccatttgctgtggacggtcgaagacatgtcttttgatttttttggtgcttggttacctttgtgggcaatctcgcggccaa

SEQ ID NO: 121 - protein, 28 aa, Lipomyces starkeyi
MTICCGRSKTCLLIFLVLGYLCGQSRGQ

SEQ ID NO: 122 - DNA, 63 bp, Lipomyces starkeyi
  source 1..63: mol_type = genomic DNA
atgaagttcttcaccatcatctccgctactctcatcctcctgctacggcaagtcattgctgac

SEQ ID NO: 123 - protein, 21 aa, Lipomyces starkeyi
MKFFTIISATLILLLRQVIAD

SEQ ID NO: 124 - DNA, 60 bp, Lipomyces starkeyi
  source 1..60: mol_type = genomic DNA
atgcggttcccctctgcgattgctgcacttgcgtcggtcatcggctgcgtgtcagccatt

SEQ ID NO: 125 - protein, 20 aa, Lipomyces starkeyi
MRFPSAIAALASVIGCVSAI

SEQ ID NO: 126 - DNA, 51 bp, Lipomyces starkeyi
  source 1..51: mol_type = genomic DNA
atgaaattcatcgttgtcgtcaccactttctcggcgattgcttcggctctc

SEQ ID NO: 127 - protein, 17 aa, Lipomyces starkeyi
MKFIVVVTTFSAIASAL

SEQ ID NO: 128 - DNA, 60 bp, Lipomyces starkeyi
  source 1..60: mol_type = genomic DNA
atgcgactgatactttcgactctcgcactattactcgcccacgtcgctgtctccgcagcg

SEQ ID NO: 129 - protein, 20 aa, Lipomyces starkeyi
MRLILSTLALLLAHVAVSAA

SEQ ID NO: 130 - DNA, 66 bp, Lipomyces starkeyi
  source 1..66: mol_type = genomic DNA
atggtttccgtaacgacttgcatcctcacagtactgattggatttggcagttttgtcaggtcctac

SEQ ID NO: 131 - protein, 22 aa, Lipomyces starkeyi
MVSVTTCILTVLIGFGSFVRSY

SEQ ID NO: 132 - DNA, 69 bp, Lipomyces starkeyi
  source 1..69: mol_type = genomic DNA
atgtttttgctaggctcttggatttaccttctggctgtatgcatactctttgatatttccgattgcgcg

SEQ ID NO: 133 - protein, 23 aa, Lipomyces starkeyi
MFLLGSWIYLLAVCILFDISDCA

SEQ ID NO: 134 - DNA, 75 bp, Lipomyces starkeyi
  source 1..75: mol_type = genomic DNA
atgggtcacgtcttcggcgcattcacagtgcttgtgtgcgccctgtttggagtcgtcatcacagcaaacggaatc

SEQ ID NO: 135 - protein, 25 aa, Lipomyces starkeyi
MGHVFGAFTVLVCALFGVVITANGI

SEQ ID NO: 136 - DNA, 75 bp, Lipomyces starkeyi
  source 1..75: mol_type = genomic DNA
atgcgatccacttttctttttctgtttgcctttctagtcgtcttactcgtgctggaggactccaccgaggcgcaa

SEQ ID NO: 137 - protein, 25 aa, Lipomyces starkeyi
MRSTFLFLFAFLVVLLVLEDSTEAQ

SEQ ID NO: 138 - DNA, 66 bp, Lipomyces starkeyi
  source 1..66: mol_type = genomic DNA
atgctttccacacctgttatactagcttgtcagctatttctgctgttttctccagcttcggctggc

SEQ ID NO: 139 - protein, 22 aa, Lipomyces starkeyi
MLSTPVILACQLFLLFSPASAG

SEQ ID NO: 140 - DNA, 60 bp, Lipomyces starkeyi
  source 1..60: mol_type = genomic DNA
atgattgcactgattatatttctcgtcttgggatcgctaacccttcgatcaaatgccgtg

SEQ ID NO: 141 - protein, 20 aa, Lipomyces starkeyi
MIALIIFLVLGSLTLRSNAV

SEQ ID NO: 142 - DNA, 72 bp, Lipomyces starkeyi
  source 1..72: mol_type = genomic DNA
atgcgccggatatcgccctggtcctggtgcgggctggtggtcttattactcgcctggccatcactcgcagca

SEQ ID NO: 143 - protein, 24 aa, Lipomyces starkeyi
MRRISPWSWCGLVVLLLAWPSLAA

SEQ ID NO: 144 - DNA, 63 bp, Lipomyces starkeyi
  source 1..63: mol_type = genomic DNA
atgatgttattgcactttctggtagtagcacttacgacgtgggcgtcgctcggtagtgcagcg

SEQ ID NO: 145 - protein, 21 aa, Lipomyces starkeyi
MMLLHFLVVALTTWASLGSAA

SEQ ID NO: 146 - DNA, 57 bp, Lipomyces starkeyi; identical to SEQ ID NO: 114
  source 1..57: mol_type = genomic DNA

SEQ ID NO: 147 - protein, 19 aa, Lipomyces starkeyi
MKSSIILSSFLSALAVTAD

SEQ ID NO: 148 - DNA, 93 bp, Lipomyces starkeyi
  source 1..93: mol_type = genomic DNA
atgctcgcgttgtcattgccacggcaggctcttcacctcgcctttccatgcatcctgcttattatggcactttcagcttgtgtgcatgctgaa

SEQ ID NO: 149 - protein, 31 aa, Lipomyces starkeyi
MLALSLPRQALHLAFPCILLIMALSACVHAE

SEQ ID NO: 150 - DNA, 90 bp, Lipomyces starkeyi
  source 1..90: mol_type = genomic DNA
atgcgaggtgtcctagtcagagcagggacagtgatactgtttgcctcctccatcgtcacggctatcgagccttctgcagcggagccggct

SEQ ID NO: 151 - protein, 30 aa, Lipomyces starkeyi
MRGVLVRAGTVILFASSIVTAIEPSAAEPA

SEQ ID NO: 152 - DNA, 69 bp, Lipomyces starkeyi
  source 1..69: mol_type = genomic DNA
atgctgatatcgcagtgtattttcatgtgcgttgtgtttttcgccacaatcgcgttcgcgggagcagat

SEQ ID NO: 153 - protein, 23 aa, Lipomyces starkeyi
MLISQCIFMCVVFFATIAFAGAD

SEQ ID NO: 154 - DNA, 87 bp, Lipomyces starkeyi
  source 1..87: mol_type = genomic DNA
atgctgacgaagtcgctctccttacgggccgccgtcgtcaccattctcctcagtcttctgttcacagtcgcgcccgcaaacgcgttc

SEQ ID NO: 155 - protein, 29 aa, Lipomyces starkeyi
MLTKSLSLRAAVVTILLSLLFTVAPANAF

SEQ ID NO: 156 - DNA, 63 bp, Lipomyces starkeyi
  source 1..63: mol_type = genomic DNA
atggcactccttgctcgcgttacctgtctggtcttgaccaccttactggctattgctgggtgc

SEQ ID NO: 157 - protein, 21 aa, Lipomyces starkeyi
MALLARVTCLVLTTLLAIAGC

SEQ ID NO: 158 - DNA, 72 bp, Lipomyces starkeyi
  source 1..72: mol_type = genomic DNA
atgcgcgcgacagtgatccctacaatcacgattttagtgattttgatggttacgaagggagttgaggggttg

SEQ ID NO: 159 - protein, 24 aa, Lipomyces starkeyi
MRATVIPTITILVILMVTKGVEGL

SEQ ID NO: 160 - DNA, 72 bp, Lipomyces starkeyi
  source 1..72: mol_type = genomic DNA
atgaataggatactttcgttattaggcacatttgtagttctgctgagcgttctgcatgtggcagtggcacat

SEQ ID NO: 161 - protein, 24 aa, Lipomyces starkeyi
MNRILSLLGTFVVLLSVLHVAVAH

SEQ ID NO: 162 - DNA, 51 bp, Lipomyces starkeyi
  source 1..51: mol_type = genomic DNA
atggccgccttgggcgccgtcgcgactctcctttctgcagtcagcgcggcc

SEQ ID NO: 163 - protein, 17 aa, Lipomyces starkeyi
MAALGAVATLLSAVSAA

SEQ ID NO: 164 - DNA, 63 bp, Lipomyces starkeyi
  source 1..63: mol_type = genomic DNA
atgctacggtggaatctcatccttaccgcactgttcctccttgcacacgcagtctccgcctat

SEQ ID NO: 165 - protein, 21 aa, Lipomyces starkeyi
MLRWNLILTALFLLAHAVSAY

(19) United States
(12) Patent Application Publication
(10) Pub. No.: US 2026/0176661 A1
(43) Pub. Date: Jun. 25, 2026

(54) CARBOHYDROLYTIC AND LIPOGENIC RECOMBINANT YEASTS

(71) Applicant: Xylome Corporation, Madison, WI (US)

(72) Inventors: Thomas W. Jeffries, Madison, WI (US); Thomas J. Kelleher, Thousand Oaks, CA (US); David Z. Mokry, Madison, WI (US); Joanna D. Bundus, Madison, WI (US); Joshua J. Dietrich, Madison, WI (US)

(73) Assignee: Xylome Corporation, Madison, WI (US)

(21) Appl. No.: 19/126,014

(22) PCT Filed: Nov. 3, 2023
(86) PCT No.: PCT/US2023/078682

§ 371 Date: Apr. 30, 2025

Related U.S. Application Data
(60) Provisional application No. 63/422,265, filed on Nov. 3, 2022.

(51) Int. Cl. C12P 7/64; C12N 9/02; C12N 9/18; C12N 9/24; C12N 9/42; C12N 15/52; C12N 15/81

(52) U.S. Cl. CPC C12P 7/64; C12N 9/0083; C12N 9/18; C12N 9/2402; C12N 9/2437; C12N 9/248; C12N 15/52; C12N 15/815; C12Y 301/01071; C12Y 302/01055; C12Y 302/01091; C12N 2800/102

(57) ABSTRACT

Recombinant yeasts bioengineered to overexpress genes for utilization of cellulosic and hemicellulosic fibers and/or produce lipid, and methods of use thereof. The yeasts are modified to express, constitutively express, or overexpress an exo-cellobiohydrolase I, an exo-cellobiohydrolase II, an endoglucanase, an endoxylanase, or any combination thereof. The yeasts may also be modified to express, constitutively express, or overexpress a lytic polysaccharide monooxygenase, an arabionofuranosidase, a xylanases, an acetylxylan esterase, or any combination thereof. The methods include cultivating the recombinant yeasts to convert low value organic stillage byproducts such as cellulose and hemicellulose into lipids suitable for biodiesel production and other higher value uses.

### CROSS-REFERENCE TO RELATED APPLICATIONS

Priority is hereby claimed to U.S. Provisional Application 63/422,265, filed Nov. 3, 2022, which is incorporated herein by reference.

### STATEMENT REGARDING FEDERALLY SPONSORED RESEARCH

This invention was made with government support under DE-EE0008497 awarded by the U.S. Department of Energy. The government has certain rights in the invention.

### SEQUENCE LISTING

The instant application contains a Sequence Listing which has been submitted in XML format and is hereby incorporated by reference in its entirety. The XML copy, created on Nov. 1, 2023, is named PCT-231103-SEQ_LIST.xml and is 235,160 bytes in size.

### BACKGROUND

Grain ethanol plants are a potential source of unused, soluble and insoluble organic materials suitable for lipid and/or biodiesel production. In wet and dry-mill ethanol operations, cornstarch is enzymatically converted into sugar then fermented to ethanol. The process leaves behind significant amounts of com fiber and generates soluble organics as byproducts of ethanol production.

Grain ethanol plants are becoming less economical to operate due to lack of demand for ethanol and to low profit margins when grain prices are high and petroleum prices are low. Ethanol derived from grain is also criticized for having poor compatibility with fuel distribution systems, reducing the food supply, contributing to soil erosion, and releasing net CO₂ emissions that are only marginally better than gasoline. Reduced operating costs, increased process efficiency, better fuel compatibility, and higher product value and diversity could significantly improve the economics and environmental acceptability of this process.

In a conventional dry mill process, corn is milled, steamed, liquified and fermented using enzymes and yeast to produce CO₂ and ethanol. The ethanol is removed from the mash by distillation, and the remaining suspension (whole stillage) is separated by centrifugation into a solids fraction called distiller's wet grain (DWG) and a mostly liquid fraction called thin stillage (TS). The distiller's wet grain is dried to create distiller's dried grain (DDG), which can be sold directly as a feed for ruminants, such as cattle. The thin stillage is concentrated into a syrup and sprayed onto distiller's dried grain to create distiller's dried grain with solubles (DDGS), which is also suitable as a ruminant feed, but has a lower value than distiller's dried grain due to the presence of the solubles from the thin stillage. These solubles, largely glycerol and oligosaccharides, make water removal difficult, dilute the protein content and reduce the distiller's dried grain value. [10] (FIG. 1A).

Corn starch, protein, and oil constitute about 66.5%, 13.5%, 4.4% of the corn kernel, respectively on a dry weight basis. [2-4] Non-fermentable cellulosic and hemicellulosic fiber make up much of the balance. Following fermentation, sieving can recover 55 to 58% of the solid fraction, which contains about 56% neutral detergent fiber (NDF), 31% protein and 9.5% oil. [5] Composition of the sieved fraction is somewhat different from that of distiller's wet grain, which is recovered from whole stillage by centrifugation in a horizontal decanter. Distiller's wet grain is relatively high in crude protein (35%), but the sugar anhydride (i.e. fiber) component is even larger (≈40%). The ether extractives represent corn oil while water extractives represent residual thin stillage solubles (TSS) that are not removed by decantation. The polysaccharides in distiller's wet grain include 5% starch, 15% cellulose, 14% xylan and 9% arabinan for a total of about 41% of the dry weight. Microscopic cellulosic and hemicellulosic neutral detergent fiber constitute about 40% of the total organics in conventional thin stillage and distiller's wet grain; however, the fraction of corn fiber in thin stillage and distiller's wet grain can vary greatly with the process, and with the development of newer technologies to recover oil and protein, they are changing rapidly. [6] Conventional thin stillage obtained without clarification or fractionation contains about 35% protein and has a nitrogen content of ≈6 to 8% on a dry weight basis.

Likewise, thin stillage varies a great deal from one ethanol plant to another. The soluble organic content of thin stillage from three independent ethanol plants is listed in Table 1, with glycerol and oligosaccharides being the major components in all cases. [6, 22] Glycerol has been reported as the single largest component of thin stillage solubles (45%) with ethanol, arabinose and glucose making up about 5%. If lactobacillus contamination is present, lactic acid can also be a component. Glucan comprises both residual starch and cellulosic microfibrils or oligosaccharides. Together with xylan and arabinan, these comprise about 45% of thin stillage by dry weight. Corn oil and protein are also present but were not reported in this analysis. In a conventional dry mill operation, the thin stillage solubles are sprayed back onto the distiller's dried grain after the distiller's wet grain is dried. As can be seen from analyses, the addition of thin stillage solubles causes the water extractive content to go up along with ether extractives while the protein and sugar anhydride contents go down. Based on the overall composition of stillage solids, the sugar anhydrides are composed mainly of residual starch, hemicellulose, and cellulosic fibers. [5] The exact composition of the fiber component is not well studied or reported. Aside from the presence of glycerol, soluble mono and disaccharides, the profile of thin stillage insoluble components is similar to that found in distiller's wet grain.

TABLE 1
Soluble organic content of thin stillage
from three independent ethanol plants.

| Thin stillage | Plant 1 | Plant 2 | Plant 3 | Average | STDEV |
|---|---|---|---|---|---|
| Glucan (g/L) | 15.3 | 11.4 | 11.3 | 12.7 | 2.28 |
| Glucose (g/L) | 0.2 | 0.1 | 3.7 | 1.3 | 2.05 |
| Xylan (g/L) | 3.1 | 1.8 | 3.2 | 2.7 | 0.78 |
| Xylose (g/L) | 0.9 | 1 | 0 | 0.6 | 0.55 |
| Arabinan (g/L) | 1.3 | 1.1 | 1 | 1.1 | 0.15 |
| Arabinose (g/L) | 0.2 | 0.2 | 0.2 | 0.2 | 0.00 |
| Glycerol (g/L) | 15.6 | 16.7 | 17.9 | 16.7 | 1.15 |
| Acetic Acid (g/L) | 0.9 | 0.9 | 0.9 | 0.9 | 0.00 |
| Ethanol (g/L) | 0.6 | 0.5 | 0.8 | 0.6 | 0.15 |

The soluble organics in stillage constitute a highly suitable substrate for lipid production. For example, yeasts have been engineered for converting the soluble organics into lipids and other biomaterials. However, methods and tools for converting fiber byproducts from ethanol production into lipids and/or biodiesels are needed to increase fuel production and create higher value products from underutilized organic byproducts.

### SUMMARY OF THE INVENTION

The invention addresses the aforementioned needs by providing recombinant yeasts. The recombinant yeasts in preferred versions are carbohydrolytic (celluloytic and/or hemicellulolytic) and lipogenic and can be used, for example, in methods for converting cellulosic and/or hemicellulosic fibers into lipids and other valuable biomaterials.

One aspect of the invention is directed to recombinant yeasts. The recombinant yeasts are modified to express, constitutively express, or overexpress any one or more of an exo-cellobiohydrolase I, an exo-cellobiohydrolase II, an endoglucanase, and an endoxylanase. In some cases, the recombinant yeasts are modified to further express, constitutively express, or overexpress any one or more additional enzymes selected from the group consisting of a lytic polysaccharide monooxygenase (LPMO), an arabinofuranosidase, a xylanase, and an acetylxylan esterase. In some cases, the recombinant yeasts are modified to further express, constitutively express, or overexpress any one or more additional enzymes selected from the group consisting of a diacylglycerol acyltransferase, a malic enzyme, and a glycerol-3-phosphate acyltransferase. In some cases, the recombinant yeasts are modified to further express, constitutively express, or overexpress any one or more additional enzymes selected from the group consisting of a glycerol kinase and a glycerol-3-phosphate dehydrogenase. The recombinant yeasts of the invention can be used for converting cellulosic and/or hemicellulosic fibers into fatty acids suitable for biodiesel, a palm oil biosimilar, or other metabolic products.

Also provided herein are methods of processing a substrate. Some versions comprise contacting the substrate comprising a first organic with the yeast of the invention, wherein the yeast consumes the first organic and produces a second organic. In some versions, first organic comprises one or more of cellulose, hemicellulose, and glycerol. In some versions, the second organic comprises lipid. Some methods comprise contacting a substrate comprising cellulose and/or hemicellulose by contacting the substrate with the recombinant yeasts of the invention. The recombinant yeasts convert the cellulose and/or hemicellulose into corresponding mono- or disaccharides and then to lipids. The method may find use for converting organics in grain ethanol distillation stillage into lipids and other metabolic products. In an exemplary process, the recombinant yeasts are first grown on glycerol and free sugars present in the stillage and then the pretreated cellulosic and hemicellulosic sugars are added to increase lipid content in the cells.

The technology described herein can increase profit margins for grain ethanol producers by making higher value and more diverse byproducts. The technology also has the added benefits of decreasing thin stillage viscosity, increasing protein production, reducing organic load from wastewaters, reducing natural gas-based processing expenses, and, in some cases, releasing a larger fraction of corn oil from the dried grain solids. Hence, the technologies would be of interest to several industries by providing a means to diversify products and increase the supply of high energy density renewable fuels while at the same time reducing environmental pressures.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1B. Simplified diagram of com dry milling. FIG. 1A. In traditional com dry milling, thin stillage is evaporated into a syrup that is sprayed onto distiller's dry grain (DDG) to form distiller's dry grain with solubles (DDGS), decreasing its value. FIG. 1B. In an exemplary process of the invention, the thin stillage is also concentrated, but is then used in a secondary fermentation with a recombinant yeast of the invention to allow for product diversification by yielding biomass, lipids, and enzymes. The DDG product is not converted to DDGS, allowing its value to be retained. TS, thin stillage; CTS, clarified TS; DWG, distiller's wet grain; DDG, distiller's dry grain; DDGS, DDG with solubles.

FIG. 2. Factory block diagram of an exemplary process of the invention integrated into an ethanol plant. The feedstock for the recombinant yeasts of the invention is derived from thin stillage and fiber from com stover, DDG, or other sources. The resulting lipid is extracted and converted to biodiesel, and waste glycerol from the conversion to biodiesel is recycled into the secondary fermenter. Enzymes produced during the fermentation are recovered for sale or reuse, and proteins without a specific end use are recycled and used as a nitrogen source. Water is recovered wherever possible.

FIG. 3. Accumulation of lipid in the lipogenic (XYL403) strain. Lipid titer (g/L) was monitored over the course of 160 hours in a bioreactor fed with either an enzymatic hydrolysate of com stover (from the National Renewable Energy Laboratory (NREL)) (black line), or a simulated hydrolysate that contained just the sugar components of the com stover hydrolysate (dashed line). The final lipid titers were similar in both cases. The XYL403 strain is engineered to be lipogenic, but it is not cellulolytic.

FIG. 4. Evaluation of secretion signals in Lipomyces starkeyi. Five sequences were identified that drive secretion of a reporter protein (GFP) into the culture media when adhered to the N-terminus, as deemed by an increase of fluorescence relative to the wild-type culture media supernatant. The sequences were derived from an α-amylase (AMY), a dextranase (DEX), an alkaline extracellular protease (PRO), a subtilisin like peptidase (PEP), and an α-glucosidase (GLU).

FIGS. 5A and 5B. Establishing a screening assay for exo-cellobiohydrolase specific activity. Two exo-cellobiohydrolases (I or II) with functional secretion signals were codon optimized and cloned into L. starkeyi under constitutive promoters. Following culture outgrowth, supernatants obtained by centrifugation underwent preliminary screening based on either cleavage of the fluorescent molecule 4-methylumbelliferyl-beta-D-cellotetraoside (4-MUC4) (FIG. 5A) or by absorbance at 405 nm of 2-chloro-4-nitrophenyl-beta-D-tetraoside (CPNPG4) (FIG. 5B). Both techniques were successful in detecting exo-cellobiohydrolase activity. Despite the lower sensitivity of the CPNPG4 method, it is more cost effective for larger (96 sample) screens.

FIGS. 6A and 6B. Establishing an assay for exo-cellobiohydrolase I specific activity. Three CBH1 type enzymes (TrCBH1, AnCBHB, and chimeric TeCD/TrCBM) with secretion signals were codon optimized and cloned into L. starkeyi under constitutive promoters. Following culture outgrowth, supernatants obtained by centrifugation were evaluated for their ability to cleave either 2-chloro-4-nitrophenyl-beta-D-tetraoside (CPNPG4) (FIG. 6A) or 4-nitrophenol β-lactoside (4-NPL) (FIG. 6B) colorimetric substrates based on absorbance at 405 nm. Large standard deviations result from different expression levels of the enzymes in each transformant due to integration within unique genetic loci. The CPNPG4 based assay allowed differential detection of activity between the CBH1 enzymes, with the highest activity derived from the chimeric TeCD/T/CBM construct, followed by the AnCBHB construct. Only marginal activity was detected against the 4NPL substrate from the chimeric TeCD/7/CBM enzyme. No activity was detected in either assay from supernatants derived from strains transformed with the TrCBH1 overexpression cassette.

FIG. 7. Competition screen between the top 4 strains secreting either AnCBHB or chimeric TeCD/TrCBM. Activity was detected based on the ability of culture supernatants to cleave 2-chloro-4-nitrophenyl-beta-D-tetraoside (CPNPG4). A higher intrinsic activity of the chimeric TeCD/TrCBM enzyme over AnCBHB was confirmed using this substrate.

FIG. 8. Establishing an assay for large-scale screening of endoglucanase activity. An endoglucanase from Aspergillus niger (AnEglA) was codon optimized and cloned into L. starkeyi under a constitutive promoter. Following culture outgrowth, the supernatant was obtained by centrifugation and evaluated for the ability to cleave 2-chloro-4-nitrophenyl-beta-D-cellotetraoside (CPNPG4). Activity is indicated by an increase in absorbance at 405 nm relative to the wild-type strain. These data support the use of this methodology for large scale screening, such as in a 96 well plate.

FIGS. 9A and 9B. Ranking comparison of AnEglA transformants by different screening methodologies. Supernatants of L. starkeyi AnEglA transformants and a 1:20,000 dilution of a CTec2 enzyme cocktail were screened by BPNPG5 (diamonds), CPNPG4 (crosses), and BzMUG3 (Xs), and assigned a numerical ranking based on performance by screening methodology, with lower numbers representing higher performance. The rankings of each transformant were then averaged and plotted, demonstrating that the relative activity of each transformant is similar across all screening methodologies, and each assay is capable of identifying identical superior transformants.

FIGS. 10A-10C. Variance distribution of sample replicates derived from top cellulase transformants by different screening methodologies. Top transformant superatants secreting cellulases AnEglA (far left), AnEglB (left), ApCel5A (right), and TrEGII (far right) were screened by BzMUG3 (FIG. 10A), BPNPG5 (FIG. 10B), and CPNPG4 (FIG. 10C) based assays and plotted. The variance of each replicate is very low, demonstrating the reliability of the assay to consistently segregate transformants based on activity.

FIG. 11. Top endoglucanase transformant activity evaluated by different substrates and normalized to wild-type. Supernatants of top transformant cellulases AnEglA (black, far left bar in each group), AnEglB (dark gray, middle left bar in each group), TrEGII (light gray middle right bar in each group), and ApCel5A (white far right bar in each group) were screened by BzMUG3, BPNPG5, and CPNPG4 and normalized to wild-type activity (Relative Activity=1). The relative activity of each cellulase against each substrate can be evaluated in this manner, providing insight into the overall intrinsic activity of each enzyme.

FIGS. 12A-12C. Comparison of endoxylanase activity from xylanase transformants based on cleavage of Xy1X6. FIG. 12A. Samples derived from (left to right) supernatants of L. starkeyi ThXYN2 transformants, AnXYNA transformants, wild-type cells, or a YPD medium blank, were screened by Xy1X6 and plotted according to their absorbance at 405 nm. FIG. 12B. Culture supernatants (shaded gray) of triplicate top transformants of ThXYN2, or wild-type L. starkeyi, or dilutions of the CTec2 or HTec2 commercial enzyme cocktails (shaded black) were screened by Xy1X6 as in (FIG. 12A), averaged, and plotted. The results demonstrate that the relative activity of each replicate is similar, and that the XylX6 based assay is capable of consistently identifying superior transformants. FIG. 12C. Comparison of endoxylanase activities of the wild-type (gray square), top ThXYN2 (gray diamond), T/XYND (black circles), 7/XYNB (black triangles), and FgXYNC (black squares) transformants. The ThXYN2 and 7/XYND enzymes presented the highest activity, followed by marginal activity observed in transformants expressing 7/XYNB and no activity in transformants of FgXYNC.

FIGS. 13A-13F. Stability evaluation of cellulases heterologously expressed and secreted in L. starkeyi. FIGS. 13A-13E. Cultures expressing the indicated cellulase AnEglA (FIG. 13A), AnEglB (FIG. 13B), ApCel5a (FIG. 13C), chimeric TeCD/TrCBM (FIG. 13D), or TrEGII (FIG. 13E) were grown to saturation in YPD for 3 days from which a supernatant sample was evaluated for activity (black circles). After 4 days at room temperature, the same supernatant was again evaluated for activity (gray diamonds). A second supernatant sample (gray triangles) was obtained from the original culture (now 7 days old) and also evaluated for activity. FIG. 13F. AnCBHB activity was evaluated from various transformant supernatants using the same assay, but in an end-point experiment without generating a second supernatant sample. All activity analyses were based on the ability to engage 2-chloro-4-nitrophenyl-beta-D-cellotetraoside (CPNPG4) as a substrate.

FIGS. 14A and 14B. Enhanced activity of cellulase and hemicellulase enzymes expressed and secreted in XYL403. The strains indicated were grown for 3 days in YPD from which the supernatants were evaluated by either CNPG4 (FIG. 14A) or XylX6 (FIG. 14B). Only the top 5 transformants were evaluated of each cassette, whereas all other strains were isogenic and evaluated in triplicate. FIG. 14A. With exception of the chimeric CBH1 enzyme cassette (TeCD/TrCBM), all other cellulases transformed into the XYL403 background (white rectangles) had higher activity, likely due to enhanced secretion of the enzymes, over those expressed in the wild-type (black rectangles) strain. FIG. 14B. The xylanase ThXYN2 cassette also displayed higher activity, likely due to enhanced secretion, in the XYL403 background (white rectangle) compared to the wild-type or cellulolytic CEL3008 background (black rectangles), indicating the effect is not an artifact of multiple transformations, but specific to XYL403.

FIGS. 15A-15B. Example of phenotypic and PCR genotyping confirmation of resistance marker removal and cellulolytic cassette retention in cured strain candidates. FIG. 15A. Two cured strain candidates of CEL3007 were plated onto Yeast Peptone+2% Dextrose solid media (YPD) or YPD plus the relevant antibiotic to the excised resistance marker (hygromycin, zeocin, or nourseothricin) and allowed to grow for several days at 30° C. None of the cured candidates grew on any of the antibiotic containing plates, indicating proper excision of the resistance marker cassette. FIG. 15B. PCR genotyping for the NAT resistance marker and the chimeric TeCD/TrCBM exo-cellobiohydrolase I cassette was performed on the wild-type, uncured CEL3007 and CEL3008 strains, and two cured candidates from each. Amplification of the NAT marker was only evident in the uncured CEL3007 and CEL3008 strains, indicating it was no longer present in the genome of the cured strains. The TeCD/TrCBM cassette was present in all but the wild-type samples, indicating it was retained in the cured strains.

FIG. 16. Ability of pooled transformant supernatants to deconstruct cellulose based filter paper. Culture superatants derived from wild-type cells (left tube) or transformants expressing single cellulases (right tube) were pooled and incubated at 30° C. for 4 days. A very mild vortex treatment for ˜1 second was then employed to resuspend any particulate material present for imaging purposes. The ability of the pooled cellulase samples to deconstruct filter paper is evident.

FIGS. 17A-17B. Filter paper deconstruction by supernatants of 3rd-generation cellulase-expressing strains. FIG. 17A. Strains secreting the indicated endoglucanase, exo-cellobiohydrolase II, and exo-cellobiohydrolase I were grown for 8 days in YP+4% Xylose medium at 28° C. Culture supernatants were then obtained by centrifugation (to remove the cells) and incubated with a 1×3.5 cm strip of Whatman No. 1 filter paper for an additional 6 days at 28° C., at which point the images were captured and scored for filter paper degradation. “TeTrCBH1” refers to the TeCD/TrCBM) enzyme. FIG. 17B. HPLC analysis using an HPX-87H column was performed on a subset of samples (wild-type and CEL3005-CEL3008) prepared in an identical or highly similar manner. The results demonstrate release of cellobiose from filter paper in the culture supernatants derived from the cellulolytic strains, as indicated by the peak at 7.15 minutes, which is absent in the wild-type derived sample. The amount of cellobiose released moderately correlates with the observed amount of deconstructed filter paper.

FIG. 18. Solubilization of DMR corn stover as measured by a Neutral Detergent Fiber based assay from selected strains' secretomes or a commercial cocktail. Quantitative degradation of DMR corn stover was measured after incubation for 8 days at 50° C. and corrected to an uninoculated control. CHTec is an equivolumetric suspension of Cellic™ CTec2 and HTec2. The commercial cocktail was capable of solubilizing more than 70% of the material, compared to 10% from the CEL4001 strain, the latter of which likely contains fewer enzyme classes in a lower concentration.

FIGS. 19A-19B. Degradation of various fiber containing substrates from enzymes secreted in the CEL4001 strain. FIG. 19A. Qualitative degradation of substrates (filter paper, top; DMR com stover, middle; stillage fiber, bottom) after incubation for 24 hours at 30° C. in wild-type (left) or CEL4001 (right) culture supernatants. FIG. 19B. Quantitative degradation of filter paper and DMR com stover after incubation for 5 days at 30° C. and corrected to the wild-type background and processing losses. Both substrates underwent a corrected mass loss between 12-15%.

FIGS. 20A-20C. Metrics of filter paper solubilization in different strain supernatants and temperatures. Supernatants derived from saturated wild-type, CEL3008, and CEL4002 cultures were incubated at 30° C. or 50° C. and evaluated by 3,5-dinitrosalicylic acid (DNS) absorbance (FIG. 20A), percent material loss based on insoluble mass remaining (FIG. 20B), and high-performance liquid chromatography (HPLC) (FIG. 20C) using an HPX-87H column for the presence of cellobiose and glucose. In all cases, the highest activity was observed in the CEL4002 derived sample at 50° C. Evidence of a native secreted β-glucosidase is also present.

FIGS. 21A-21B. Lipid titers of wild-type and engineered CEL4001 cultured on media containing stillage fiber derived from commercial ethanol manufacturing. FIG. 21A. Wild-type, CEL4001, and an uninoculated media blank (50 mL each) were assessed in 250 mL baffled shake flasks containing 5% of a stillage fiber-based substrate in triplicate. The wild-type and CEL4001 samples were inoculated from starter cultures at 50% final volumes. Cultures were incubated at 30° C. for 96 hours before undergoing gravimetric lipid analysis. The engineered strain, CEL4001, resulted in twice as much lipid compared to the wild-type sample, but considering some lipid is contributed by the substrate, the actual difference is much higher (closer to 3×). FIG. 21B. An enzyme rich culture broth derived from wild-type or CEL4001 cultures grown on nitrogen rich media was isolated and incubated with DMR com stover to release sugars. After 5 days of saccharification at 50° C., the solids portion of the slurry was removed, and the resulting suspension inoculated with wild-type or CEL4001 cells and cultured for two days. A parallel experiment was also performed in shake flasks using the clarified slurry as a culture broth. In both cases, the engineered CEL4001 strain accumulated 4.8 and 3.3 times more lipid than the wild-type strain.

FIG. 22. Arabinofuranosidase activity of the CEL3427 strain and three CEL4408 strains with AnAxhA (α-L-arabinofuranosidase) integrations. Arabinofuranosidase activity was measured using an azo-wheat arabinofuranoxylanase enzyme activity assay. Enzyme broth derived from strains containing AnAxhA were compared with control CEL3427 enzyme broth. All enzyme broths were incubated on the azo-wheat arabinoxylanase based substrate for 1 hour in a 30° C. shaker. Strains exhibiting arabinofuranosidase activity degrade the substrate and release a blue dye, increasing the OD at 590 nm.

FIG. 23. Exo-cellobiohydrolase activity of five CEL4001 strains with 7rCEL61B (LPMO) integrations.

FIGS. 24A-24E. Carbohydrolytic activities of extracellular enzyme preparations from the XYL403, CEL3427, CEL4001, CEL5001, and CEL5401 strains on various fiber sources or a dye-labeled substrate.

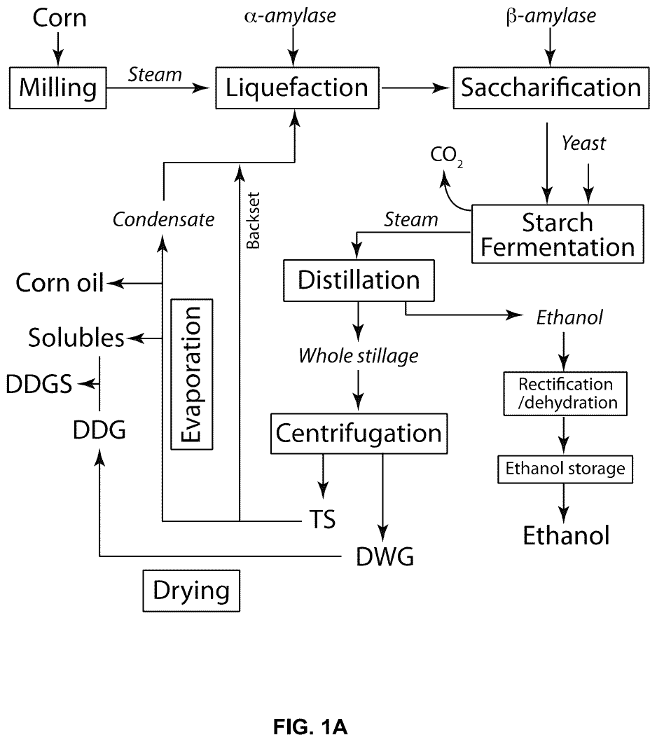

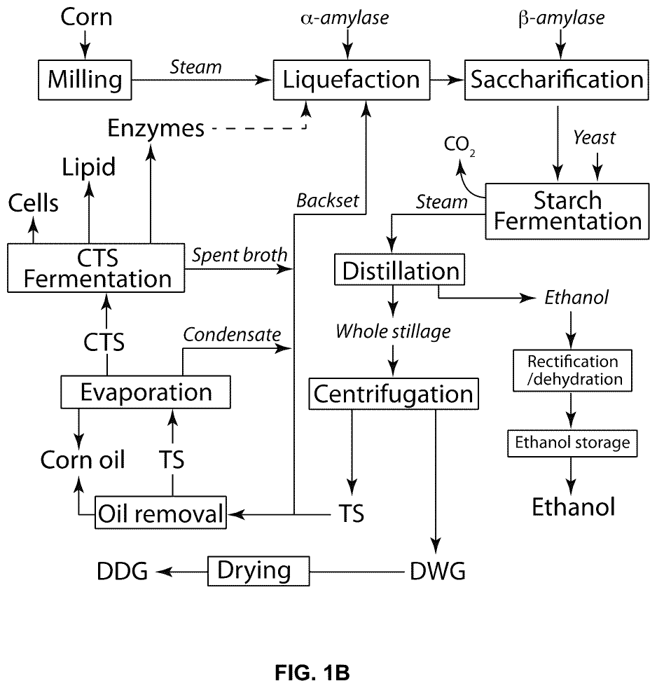

### DETAILED DESCRIPTION OF THE INVENTION

An aspect of the invention encompasses recombinant yeasts. The recombinant yeasts are preferably engineered to have enhanced carbohydrolytic and/or lipogenic activities with respect to their native counterparts. “Carbohydrolytic” as used herein refers to the ability to break down complex carbohydrates into simpler molecules. Two preferred types of carbohydrolytic activities for the recombinant yeasts of the invention include cellulolytic and hemicellulolytic activities. “Cellulolytic” refers to the ability to hydrolyze or otherwise break down cellulose. “Hemicelluloltyic” refers to the ability to hydrolyze or otherwise break down hemicellulose.

The recombinant yeasts are preferably derived from lipogenic yeasts. Lipogenic yeasts (also known as oleaginous yeasts) have been recognized for more than 50 years. They are defined as those that accumulate lipids in intracellular oil bodies to greater than 20% of their dry mass. In some yeasts, lipids have been reported to account for up to 71% of the cell's total biomass. [37] Out of the 1200 to 1500 known yeast species, only a fraction qualifies as lipogenic. Lipomyces starkeyi was among the earliest lipogenic yeasts studied. [38] Other known lipogenic yeasts include Yarrowia lipolytica. [39] and species in the genera of Rhodotorula, Cryptococcus, [40] Candida, Trichosporon. [41] Rhodosporidium, Sporidiobolus, Sporodobolomyces, and various other ascomyceteous and basidiomycete genera amounting for a total of about 100 species. [42]

Lipogenic yeasts belong to the larger taxonomic groups of filamentous ascomyceteous and basidiomycetous fungi. Exemplary lipogenic yeasts include yeasts from the genus Lipomyces, such as L. starkeyi, L. anomalus, L. arxii, L. chichibuensis, L. doorenjongii, L. japonicus, L. kockii, L. kononenkode, L. lipofer, L. mesembrius. L. oligophaga, L. orientalis, L. smithiae. L. spencermartinsiae, L. suomiensis. L. tetrasporus, L. yamadae, L. yarrowii, and L. Sp.; yeasts from the genus Yarrowia, such as Y. lipolytica, Y. bubula, Y. deformans, Y. divulgata, Y. keelungensis, Y. porcina, Y. yakushimensis, and Y. Sp.; yeasts from the genus Candida, such as C. Sp.; yeasts from the genus Hansenula, such as H. polymorpha, yeasts from the genus Cunninghamella, such as S. bigelovii sp nov CGMCC 8094, S. echinulate, S. blakeslecana JSK2, and S. Sp. Salicorn 5; yeasts from the genus Mortierella, such as M. alpina, M. isabellina, and M. Sp.; yeasts from the genus Rhodosporidium, such as R. toruloides, R. babjevae, R. diobovatum, R. fluviale, R. kratochvilovae, R. paludigenum, R. sphaerocarpum. R. araucariae. R. colostri, R. dairenensis, R. graminis. R. lusitamae, and R. mucilaginosa; yeasts from the genus Sporidiobolus, such as S. johnsonii, S. pararoseus, S. ruineniae, S. ruineniae, and S. salmonicolor; yeasts from the genus Sporobolomyces, such as S. bannaensis, S. beijingensis, S. carnicolor, S. metaroseus, S. odoratus, S. poonsookiae, S. singularis, and S. inositophilus; yeasts from the genus Occultifur, such as O. externus; yeasts from the genus Rhodotorula, such as R. bogoriensis, R. hylophila, R. glutinis, and R. rhodochrous; yeasts from the genus Trichosporon, such as T. fermentans, T. oleaginosus ATCC 20509, and T. cutaneum; and yeasts from the genus Cryptococcus, such as C. curvatus and C. Sp.

Certain filamentous fungi and unicellular algae can also be lipogenic. These include filamentous fungi from the genus Aspergillus, such as A. nidulans, and from the genus Mucor, such as M. circinelloides and M. rouxit. Lipogenic algae include species from the genus Scenedesmus, such as S. quadricauda.

Nontraditional lipogenic yeasts have an innate ability to convert poorly metabolized wastes from ethanol fermentation into lipids, protein, and enzymes. Some lipogenic yeasts naturally make large amounts of lipids from a wide variety of carbon sources, and this prodigious capacity renders them amenable to many bioprocessing applications.

Lipomyces starkeyi is a particularly preferred lipogenic yeast in this regard. L. starkeyi can utilize many different substrates, including the oligosaccharides and sugars found in both agricultural waste products and the hydrolysates of lignocellulosic material. [43, 16, 21]

L. starkeyi yeast maintains a basal lipid content that increases throughout fermentation, which already meets or exceeds that of any other native lipogenic yeast or alga. The lipid profile produced by L. starkeyi is remarkably similar to that of palm oil, one of the most common biodiesel feedstocks, which indicates that a biodiesel produced from this species naturally has desirable fuel properties. [12]

L. starkeyi is among the yeasts that can metabolize glucose, xylose and cellobiose, which are the main sugars released from the hydrolysis of lignocellulosic materials. [44] With L. starkeyi, optimal lipid production is attained when growing on a 2:1 mixture of glucose and xylose, the same ratio found in enzymatic hydrolysates. Some strains of L. starkeyi also produce lipid from glycerol.

The lipid profile of Lipomyces is similar to that of palm oil, [12] which is important for both food and fuel production. By developing technology that uses Lipomyces to produce biofuels from the hydrolysates of agricultural cellulosic residues as an alternative to seed-based oils, the present invention provides for generating fuel from a renewable, environmentally benign resource that does not compete with food production. Furthermore, biodiesel derived from non-food sources such as lignocellulosic hydrolysate is advantageous because it meets the criteria delineated under Renewable Fuel Standard 2 (RFS2), which mandates the increased use of advanced cellulosic biofuels.

Lipid accumulation typically occurs when a readily assimilated carbon source is present in excess and nitrogen is limiting. [45, 46] For example, when L. starkeyi transitions into a nitrogen-limited environment the biosynthetic pathways dependent on abundant nitrogen shut down and lipogenesis becomes the dominant metabolic feature of the cell. The cells continue to assimilate carbon, and in the absence of new cell growth, they store it as triacylglycerols. The most readily assimilated lipogenic carbon source is typically glucose. [40] and xylose has been reported to increase lipid accumulation even more. [16, 21] Other substrates include cellobiose, glycerol, oligosaccharides, various industrial organic byproducts and hydrolysate from non-edible cellulosic feedstocks. [47] The yeasts engineered herein are capable of producing lipid when glucose is limited, and carbon organics and nitrogen are in abundance.

The recombinant yeasts of the invention comprise one or more recombinant nucleic acids configured to express one or more enzymes. The one or more recombinant nucleic acids are preferably configured to constitutively express or to overexpress the one or more enzymes.

The one or more recombinant nucleic acids preferably comprise one or more recombinant genes configured to constitutively express or to overexpress the one or more enzymes. If a cell endogenously expresses a particular enzyme, the nucleic acid expressing that enzyme may be modified to exchange or optimize promoters, exchange or optimize enhancers, or exchange or optimize any other genetic element that results in increased or constitutive expression of the enzymes. Alternatively or additionally, one or more additional copies of a gene or coding sequence thereof may be introduced to the cell for enhanced expression of the enzymes. If a cell does not endogenously express a particular enzyme, one or more copies of a recombinant nucleic acid configured to express that enzyme may be introduced to the cell for expression of the enzyme. The recombinant nucleic acid may be incorporated into the genome of the cell or may be contained on an extra-chromosomal plasmid. Techniques for genetic manipulation are described in further detail below. The genetically modified yeasts of the invention are also referred to herein as “recombinant,” “engineered,” or “bioengineered” yeasts, or other designations.

The recombinant yeasts of the invention may comprise one or more recombinant genes configured to express any one or more of the following enzymes in any combination: an exo-cellobiohydrolase I, an exo-cellobiohydrolase II, an endoglucanase, and an endoxylanase. The one or more recombinant nucleic acids preferably comprise one or more recombinant genes configured to express the above-referenced enzymes.

For example, the recombinant yeasts of the invention may comprise one or more recombinant genes configured to express an exo-cellobiohydrolase I alone or with any one or more of an exo-cellobiohydrolase II, an endoglucanase, and an endoxylanase in any combination.

The recombinant yeasts of the invention may comprise one or more recombinant genes configured to express an exo-cellobiohydrolase II alone or with any one or more of an exo-cellobiohydrolase 1, an endoglucanase, and an endoxylanase in any combination.

The recombinant yeasts of the invention may comprise one or more recombinant genes configured to express an endoglucanase alone or with any one or more of an exo-cellobiohydrolase I, an exo-cellobiohydrolase II, and an endoxylanase in any combination.

The recombinant yeasts of the invention may comprise one or more recombinant genes configured to express an endoxylanase alone or with any one or more of an exo-cellobiohydrolase I, exo-cellobiohydrolase II, and an endoglucanase in any combination.

Exo-cellobiohydrolases 1 include enzymes falling under Enzyme Commission (EC) number 3.2.1.150. Exemplary exo-cellobiohydrolases 1 that may be expressed include 7/CBH1 (positions 19-514 of SEQ ID NO:2) encoded by TrCBH/(positions 55-1545 of SEQ ID NO:1) from Trichoderma reesei, and AnCBHB (positions 19-530 of SEQ ID NO:6) encoded by AnCBHB (positions 55-1593 of SEQ ID NO:5) from Aspergillus niger. Other exemplary exo-cellobiohydrolases 1 include TeCD/TrCBM (positions 19-516 of SEQ ID NO:4), a chimeric enzyme encoded by the corresponding nucleic acid (positions 55-1551 of SEQ ID NO:3) from Trichoderma reesei and Talaromyces emersonit.

Exo-cellobiohydrolases II include enzymes falling under EC number 3.2.1.91. Exemplary exo-cellobiohydrolases II that maybe expressed include TrCBH2 (positions 25-471 of SEQ ID NO:8) encoded by TrCBH2 (positions 73-1416 of SEQ ID NO:7) from Trichoderma reesei, and A/CBH2 (positions 25-425 of SEQ ID NO:10) encoded by A/CBH2 (positions 73-1278 of SEQ ID NO:9) from Aspergillus flavus.

Endoglucanases include enzymes falling under EC number 3.2.1.4. Exemplary endoglucanases that maybe expressed include AnEglA (positions 32-254 of SEQ ID NO:12) encoded by AnEglA (positions 94-764 of SEQ ID NO: 11) and AnEglB (positions 32-344 of SEQ ID NO:14) encoded by AnEglB (positions 94-1033 of SEQ ID NO:13), both from Aspergillus niger. Other exemplary endoglucanases include ApCel5A (positions 32-441 of SEQ ID NO:16) encoded by ApCel5A (positions 94-1324 of SEQ ID NO:15) from Aureobasidium pullulans, and TrEGII (positions 32-428 of SEQ ID NO:18) encoded by TrEGII (positions 94-1285 of SEQ ID NO: 17) from Trichoderma reesei.

Endoxylanases include enzymes falling under EC numbers 3.2.1.8, 3.2.1.32, and 3.2.1.136. Exemplary endoxylanases that maybe expressed include AnXYNA (positions 25-329 of SEQ ID NO:20) encoded by AnXYNA (positions 73-990 of SEQ ID NO:19) from Aspergillus niger, ThXYN2 (positions 25-226 of SEQ ID NO:22) encoded by ThXYN2 (positions 73-681 of SEQ ID NO:21) from Trichoderma harzianum, FgXYNC (positions 25-332 of SEQ ID NO:24) encoded by FgXYNC (positions 73-999 of SEQ ID NO:23) from Fusarium graminearum, TXYNB (positions 25-267 of SEQ ID NO:26) encoded by TAXYNB (positions 73-804 of SEQ ID NO:25) from Talaromyces funiculosus, and TAXYND (positions 25-412 of SEQ ID NO:28) encoded by T/XYND (positions 73-1239 of SEQ ID NO:27) from Talaromyces funiculosus.

The recombinant yeasts of the invention may further comprise one or more recombinant genes configured to express any one or more additional enzymes selected from the group consisting of a lytic polysaccharide monooxygenase, an arabinofuranosidase, a xylanase, and an acetylxylan esterase.

For example, the recombinant yeasts of the invention may further comprise one or more recombinant genes configured to express a lytic polysaccharide monooxygenase alone or with any one or more of an arabinofuranosidase, a xylanase, and an acetylxylan esterase in any combination.

The recombinant yeasts of the invention may further comprise one or more recombinant genes configured to express an arabinofuranosidase alone or with any one or more of a lytic polysaccharide monooxygenase, a xylanase, and an acetylxylan esterase in any combination.

The recombinant yeasts of the invention may further comprise one or more recombinant genes configured to express a xylanase alone or with any one or more of a lytic polysaccharide monooxygenase, an arabinofuranosidase, and an acetylxylan esterase in any combination.

The recombinant yeasts of the invention may further comprise one or more recombinant genes configured to express an acetylxylan esterase alone or with any one or more of a lytic polysaccharide monooxygenase, an arabinofuranosidase, and a xylanase in any combination.

Lytic polysaccharide monooxygenases include enzymes falling under EC numbers 1.14.99.53, 1.14.99.54, 1.14.99.55 and 1.14.99.56. Exemplary lytic polysaccharide monooxygenases that maybe expressed include 7rCEL61A (positions 19-247 of SEQ ID NO: 30) encoded by TrCEL61A (positions 55-744 of SEQ ID NO:29) and TrCEL61B (positions 19-340 of SEQ ID NO:32) encoded by TrCEL61B (positions 55-1023 of SEQ ID NO: 31), both from Trichoderma reesei.

Arabinofuranosidases include enzymes falling under BC number 3.2.1.55. Exemplary arabinofuranosidases that maybe expressed include AxhA (positions 27-332 of SEQ ID NO: 35; positions 20-325 of SEQ ID NO:97) encoded by AxhA (positions 79-999 of SEQ ID NO: 33-34 and 96; positions 58-978 of SEQ ID NO:98) from Aspergillus niger, AbfB (positions 19-499 of SEQ ID NO:38; positions 20-500 of SEQ ID NO:100) encoded by AbfB (positions 55-1500 of SEQ ID NOs: 36-37 and 99; positions 58-1503 of SEQ ID NO:101) from Aspergillus phoenicis, AbfB (positions 19-499 of SEQ ID NO:40; positions 20-500 of SEQ ID NO: 103) encoded by AbfB (positions 55-1500 of SEQ ID NO:39; positions 58-1503 of SEQ ID NO:102) from Aspergillus niger, and an α-L-arabinofuranosidase (SEQ ID NO:42) encoded by the corresponding nucleic acid (SEQ ID NO:41) from Thermobacillus xylanilyticus.

Xylanases include enzymes falling under EC numbers 3.2.1.8, 3.2.1.32, 3.2. 1.136, and 3.2.1.156. Exemplary xylanases that maybe expressed include TcXyn30B (SEQ ID NOs: 44 and 46) encoded by TcXyn30B (SEQ ID NOs: 43, 45 and 111) and ToXyn30C (SEQ ID NOs: 48 and 50) encoded by ToXyn30C (SEQ ID NOs: 47, 49 and 112), both from Talaromyces cellulolyticus, and a glucuronoarabinoxylan endo-1,4-beta-xylanase (SEQ ID NO:52) encoded by the corresponding nucleic acid (SEQ ID NOs: 51 and 113) from Ruminiclostridium papyrosolvens.

Acetylxylan esterases include enzymes falling under EC number 3.1.1.72. An exemplary acetylxylan esterase that maybe expressed includes AxeA (SEQ ID NO:54) encoded by AxeA (SEQ ID NO:53) from Aspergillus niger.

The recombinant yeasts of the invention may further comprise one or more recombinant genes configured to express one or more additional enzymes selected from the group consisting of a diacylglycerol acyltransferase, a malic enzyme, and a glycerol-3-phosphate acyltransferase.

For example, the recombinant yeasts of the invention may further comprise one or more recombinant genes configured to express a diacylglycerol acyltransferase alone or with any one or more of a malic enzyme and a glycerol-3-phosphate acyltransferase in any combination.

The recombinant yeasts of the invention may further comprise one or more recombinant genes configured to express a malic enzyme alone or with any one or more of a diacylglycerol acyltransferase and a glycerol-3-phosphate acyltransferase in any combination.

The recombinant yeasts of the invention may further comprise one or more recombinant genes configured to express a glycerol-3-phosphate acyltransferase alone or with any one or more of a diacylglycerol acyltransferase and a malic enzyme in any combination.

Diacylglycerol acyltransferases (DGAs) include enzymes falling under EC number 2.3.1.20. Exemplary diacylglycerol acyltransferases that maybe expressed include DGA1 (SEQ ID NO:83) encoded by DGA1 (SEQ ID NO:82), and DGA2 (SEQ ID NO:85) encoded by DGA2 (SEQ ID NO:84), all derived from L. starkeyi.

Malic enzymes include enzymes falling under EC numbers 1.1.1.38, 1.1.1.39, and 1.1.1.40. An exemplary malic enzyme that maybe expressed includes ME (SEQ ID NO:87) encoded by ME (SEQ ID NO:86) from L. starkeyi.

Glycerol-3-phosphate acyltransferases include enzymes falling under EC number 2.3.1.15. An exemplary glycerol-3-phosphate acyltransferase that may be expressed includes SCT1 (SEQ ID NO:89) encoded by SCT1 (SEQ ID NO:88) from L. starkeyi.

The recombinant yeasts of the invention may further comprise one or more recombinant genes configured to express one or more additional enzymes selected from the group consisting of a glycerol kinase and a glycerol-3-phosphate dehydrogenase.

For example, the recombinant yeasts of the invention may further comprise one or more recombinant genes configured to express a glycerol kinase alone or with a glycerol-3-phosphate dehydrogenase.

The recombinant yeasts of the invention may further comprise one or more recombinant genes configured to express a glycerol-3-phosphate dehydrogenase alone or with a glycerol kinase.

Glycerol kinases include enzymes falling under EC number 2.7.1.30. An exemplary glycerol kinase that may be expressed includes GUT1 (SEQ ID NO:91) encoded by GUT1 (SEQ ID NO:90) from L. starkeyi.

Glycerol-3-phosphate dehydrogenases include enzymes falling under EC numbers 1.1.1.8, 1.1.1.94, 1.1.5.3. Exemplary glycerol-3-phosphate dehydrogenases that may be expressed include the FAD-dependent glycerol-3-phosphate dehydrogenase GUT2 (SEQ ID NO: 93) encoded by GUT2 (SEQ ID NO:92), and GPDI (SEQ ID NO:95) encoded by GPDI (SEQ ID NO:94), both from L. starkeyi.

Other suitable enzymes that may be expressed include those comprising polypeptide sequences at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the any of sequences listed above or elsewhere herein. Other suitable enzymes that may be expressed include orthologs and paralogs of the enzymes listed above. Other suitable enzymes that may be expressed include those comprising polypeptide sequences at least about 80%, at least about 85%, at least about 90%, at least about 95%, at least about 97%, or at least about 99% identical to orthologs and paralogs of the enzymes listed above. The orthologs are preferably from lipogenic yeasts, such as any of the lipogenic yeasts described herein. The recombinant gene encoding the enzymes may include introns or be devoid of introns or any or all other non-coding regions in the native gene. Any nucleotide sequences capable of expressing the polypeptide sequences encompassed herein are acceptable. Tremendous variation from the exemplary nucleotide sequences described herein is possible due to the redundancy in the genetic code and codon optimization.

It is preferred that at least one or some of the enzymes encoded by the recombinant genes are secreted from the recombinant yeast. The recombinant genes for such enzymes can accordingly encode a secretion signal sequence operably linked to the enzyme. The secretion signal sequence is in some cases can be heterologous to the enzymes. Enzymes that are preferably secreted include exo-cellobiohydrolase I, chimeric exo-cellobiohydrolase I, exo-cellobiohydrolase II, endoglucanase, endoxylanase, lytic polysaccharide monooxygenase, α-L-arabinofuranosidases including α-L-arabinofuranosidases A and B, xylanase B chains A and B, xylanase C chains A and B, glucuranoarabinoxylan endo1,4 beta-xylanase and acetylxylan esterase A.

Exemplary secretion signals that may be operably linked to the enzymes include secretion signals derived from an α-amylase (AMY: SEQ ID NO:65) encoded by the corresponding nucleic acid (SEQ ID NO:64), a dextranase (DEX; SEQ ID NO:61) encoded by the corresponding nucleic acids (SEQ ID NOs: 57-60), an alkaline extracellular protease (PRO; SEQ ID NO:56) encoded by the corresponding nucleic acid (SEQ ID NO:55), a subtilisin like peptidase (PEP; SEQ ID NO:63) encoded by the corresponding nucleic acid (SEQ ID NO:62), an α-glucosidase (GLU; SEQ ID NO:67) encoded by the corresponding nucleic acid (SEQ ID NO:66), a protein of glycoside hydrolase family 3 (GH3.1; SEQ ID NO: 105) encoded by the corresponding nucleic acid (SEQ ID NO:104), an arabinofuranosidase (AxhA: SEQ ID NO:107) encoded by the corresponding nucleic acid (SEQ ID NO:106), an arabinofuranosidase B (AbfB; SEQ ID NO:109) encoded by the corresponding nucleic acid (SEQ ID NO:108), and secretion signals derived from Lipomyces starkeyi NRRL Y11557 Protein IDs 61256 (SEQ ID NO:115), 322386 (SEQ ID NO:117), 67146 (SEQ ID NO: 119), 7314 (SEQ ID NO:121), 5250 (SEQ ID NO:123), 74374 (SEQ ID NO: 125), 75319 (SEQ ID NO:127), 6714 (SEQ ID NO:129), 338499 (SEQ ID NO:131), 338374 (SEQ ID NO:133), 5459 (SEQ ID NO:135), 962 (SEQ ID NO:137), 235 (SEQ ID NO: 139), 49224 (SEQ ID NO: 141), 76563 (SEQ ID NO:143), 6909 (SEQ ID NO:145), 61256 (SEQ ID NO: 147), 84172 (SEQ ID NO:149), 120665 (SEQ ID NO:151), 75368 (SEQ ID NO: 153), 111843 (SEQ ID NO: 155), 71087 (SEQ ID NO:157), 72441 (SEQ ID NO:159), 55318 (SEQ ID NO:161), 1422 (SEQ ID NO:163), and 66955 (SEQ ID NO:165) encoded by the corresponding nucleic acids (SEQ ID NOs: 114, 116, 118, 120, 122, 124, 126, 128, 130, 132, 134, 136, 138, 140, 142, 144, 146, 148, 150, 152, 154, 156, 158, 160, 162, and 164).

Coding sequences of the above-mentioned enzymes in the recombinant genes are preferably operably linked to a promoter. The promoter may be a constitutive promoter or an inducible promoter. The promoter can be heterologous to the coding sequence. Exemplary promoters that may be operably linked to the coding sequences of the above-mentioned enzymes include the L. starkeyi ACTI promoter (SEQ ID NO:68), the L. starkeyi CYC1 promoter (SEQ ID NO:69), the L. starkeyi PDA1 promoter (SEQ ID NO:70), the L. starkeyi ACOI promoter (SEQ ID NO:71), the L. starkeyi PDE3 promoter (SEQ ID NO:72), the L. starkeyi histone bi-directional promoter (SEQ ID NO:110), or sequence variants at least about at least about 80%, at least about 85%, at least about 90%, at least about 95%, at least about 97%, or at least about 99% identical thereto.

Coding sequences of the above-mentioned enzymes in the recombinant genes are preferably operably linked to a terminator. The terminator can be heterologous to the coding sequence. Exemplary terminators that may be operably linked to the coding sequences of the above-mentioned enzymes include the L. starkeyi ACTI terminator (SEQ ID NO:73), the L. starkeyi PDA1 terminator (SEQ ID NO:74), the L. starkeyi PDE3 terminator (SEQ ID NO:75), or sequence variants at least about at least about 80%, at least about 85%, at least about 90%, at least about 95%, at least about 97%, or at least about 99% identical thereto.

The recombinant yeasts of the invention with the modifications described herein preferably exhibit a property selected from the group consisting of increased lipid production, increased lipid secretion, increased lipid production under nitrogen-rich conditions, increased lipid yield, increased lipid secretion under nitrogen-rich conditions, increased enzyme production, increased enzyme secretion, increased carbohydrase production, increased carbohydrase secretion, increased growth rates, and/or increased organic consumption, such as increased cellulosic fiber consumption, increased hemicellulosic fiber consumption, increased glycerol consumption, and/or increased disaccharide (cellobiose and/or trehalose) consumption relative to a non-recombinant control. “Carbohydrase” refers to any enzyme capable of breaking down a carbohydrate, such as amylases, cellulases, glucosidases, xylanases, etc.

The recombinant yeasts of the invention may be genetically altered to express or overexpress any of the specific genes or gene products explicitly described herein or homologs thereof. Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Nucleic acid or gene product (amino acid) sequences of any known gene, including the genes or gene products described herein, can be determined by searching any sequence databases known in the art using the gene name or accession number as a search term. Common sequence databases include GenBank (www.ncbi.nlm.nih.gov), ExPASy (expasy.org), KEGG (www.genome.jp), among others. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 35% 40%, 45% 50%, 55%, 60%, 65%, 70%, 75%, 80%, 85%, 90%, 95%, or 99% or more, can also be used to establish homology. Accordingly, homologs of the genes or gene products described herein include genes or gene products having at least about 30%, 35%, 40%, 45%, 50%, 55%, 60%, 65%, 70%, 75%, 80%, 85%, 90%, 95%, or 99% identity to the genes or gene products described herein. Methods for determining sequence similarity percentages (e.g., BLASTP and BLASTN using default parameters) are described herein and are generally available. The homologous proteins should demonstrate comparable activities and, if an enzyme, participate in the same or analogous pathways. Homologs include orthologs and paralogs. “Orthologs” are genes and products thereof in different species that evolved from a common ancestral gene by speciation. Normally, orthologs retain the same or similar function in the course of evolution. Paralogs are genes and products thereof related by duplication within a genome. As used herein, “orthologs” and “paralogs” are included in the term “homologs.”

For sequence comparison and homology determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is a nucleic acid or amino acid sequence corresponding to the genes or gene products described herein.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described above (or other algorithms available to persons of skill) or by visual inspection.

The phrase “substantially identical” in the context of two nucleic acids or polypeptides refers to two or more sequences or subsequences that have at least about 60%, about 65%, about 70%, about 75%, about 80%, about 85%, about 90, about 95%, about 98%, or about 99% or more nucleotide or amino acid residue identity, when compared and aligned for maximum correspondence, as measured using a sequence comparison algorithm or by visual inspection. Such “substantially identical” sequences are typically considered to be “homologous”, without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, at least about 250 residues, or over the full length of the two sequences to be compared.

Terms used herein pertaining to genetic manipulation are defined as follows.

Deletion: The removal of one or more nucleotides from a nucleic acid molecule or one or more amino acids from a protein, the regions on either side being joined together.

Derived: When used with reference to a nucleic acid or protein, “derived” means that the nucleic acid or polypeptide is isolated from a described source or is at least 70%, 80%, 90%, 95%, 99%, or more identical to a nucleic acid or polypeptide included in the described source.

Endogenous: As used herein with reference to a nucleic acid molecule, genetic element (e.g., gene, promoter, etc.), or polypeptide in a particular cell, “endogenous” refers to a nucleic acid molecule, genetic element, or polypeptide that is in the cell and was not introduced into the cell or transferred within the genome of the cell using recombinant engineering techniques. For example, an endogenous genetic element is a genetic element that was present in a cell in its particular locus in the genome when the cell was originally isolated from nature.

Exogenous: As used herein with reference to a nucleic acid molecule, genetic element (e.g., gene, promoter, etc.), or polypeptide in a particular cell, “exogenous” refers to any nucleic acid molecule, genetic element, or polypeptide that was introduced into the cell or transferred within the genome of the cell using recombinant engineering techniques. For example, an exogenous genetic element is a genetic element that was not present in its particular locus in the genome when the cell was originally isolated from nature.

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Introduce: When used with reference to genetic material, such as a nucleic acid, and a cell, “introduce” refers to the delivery of the genetic material to the cell in a manner such that the genetic material is capable of being expressed within the cell. Introduction of genetic material includes both transformation and transfection. Transformation encompasses techniques by which a nucleic acid molecule can be introduced into cells such as prokaryotic cells or non-animal eukaryotic cells. Transfection encompasses techniques by which a nucleic acid molecule can be introduced into cells such as animal cells. These techniques include but are not limited to introduction of a nucleic acid via conjugation, electroporation, lipofection, infection, and particle gun acceleration.

Isolated: An “isolated” biological component (such as a nucleic acid molecule, polypeptide, or cell) has been substantially separated or purified away from other biological components in which the component naturally occurs, such as other chromosomal and extrachromosomal DNA and RNA and proteins. Nucleic acid molecules and polypeptides that have been “isolated” include nucleic acid molecules and polypeptides purified by standard purification methods. The term also includes nucleic acid molecules and polypeptides prepared by recombinant expression in a cell as well as chemically synthesized nucleic acid molecules and polypeptides. In one example, “isolated” refers to a naturally-occurring nucleic acid molecule that is not immediately contiguous with both of the sequences with which it is immediately contiguous (one on the 5′ end and one on the 3′ end) in the naturally-occurring genome of the organism from which it is derived.

Gene: Genes minimally include a promoter operationally linked to a coding sequence, and can include other elements that facilitate or regulate the transcription and/or translation of the coding sequence.

Heterologous: The term “heterologous” refers to an element in an arrangement with another element that does not occur in nature. For example, a gene or protein that is heterologous to a given cell is a gene or protein that does not occur in the cell in nature. A promoter that is heterologous to a given coding sequence is a promoter that is not operably linked to the coding sequence in nature. A secretion signal sequence that is heterologous to a given protein (such as an enzyme) is a secretion signal sequence that is not operably linked with the protein in nature.

Nucleic acid: Encompasses both RNA and DNA molecules including, without limitation, cDNA, genomic DNA, and mRNA. Nucleic acids also include synthetic nucleic acid molecules, such as those that are chemically synthesized or recombinantly produced. The nucleic acid can be double-stranded or single-stranded. Where single-stranded, the nucleic acid molecule can be the sense strand, the antisense strand, or both. In addition, the nucleic acid can be circular or linear.

Operably linked: A first element is operably linked with a second element when the first element is placed in a functional relationship with the second element. For instance, a promoter is operably linked to a coding sequence if the promoter affects the transcription or expression of the coding sequence. A secretion signal sequence is operably linked to a protein (such as an enzyme) when the secretion signal sequence affects secretion of the protein from a cell.

Overexpress: When a gene is caused to be transcribed at an elevated rate compared to the endogenous or basal transcription rate for that gene. In some examples, overexpression additionally includes an elevated rate of translation of the gene compared to the endogenous translation rate for that gene. Methods of testing for overexpression are well known in the art, for example transcribed RNA levels can be assessed using RT-PCR and protein levels can be assessed using SDS-PAGE gel analysis.

Recombinant: A recombinant nucleic acid or polypeptide is one comprising a sequence that is not naturally occurring. A recombinant gene is a gene that comprises a recombinant nucleic acid sequence, is present within a cell in which it does not naturally occur, and/or is present in a different locus (e.g., genetic locus or on an extrachromosomal plasmid) within a particular cell than in a corresponding native cell. A recombinant cell (such as a recombinant yeast) is one that comprises a recombinant nucleic acid, a recombinant gene, or a recombinant polypeptide.

Vector or expression vector: An entity comprising a nucleic acid molecule that is capable of introducing the nucleic acid, or being introduced with the nucleic acid, into a cell for expression of the nucleic acid. A vector can include nucleic acid sequences that permit it to replicate in the cell, such as an origin of replication. A vector can also include one or more selectable marker genes and other genetic elements known in the art. Examples of suitable vectors are found below.

Carbohydrate active enzymes: Enzymes involved in the assembly, modification, and breakdown of complex carbohydrates and glycoconjugates. These broadly comprise the superfamilies of glycosyl transferases (GT), glycoside hydrolases (GH), carbohydrate esterases (CE), polysaccharide lyases (PL), auxiliary activities (AA), and carbohydrate-binding modules (CBMs).

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below.

Exogenous nucleic acids can be introduced stably or transiently into a cell using techniques well known in the art, including electroporation, lithium acetate transformation, calcium phosphate precipitation, DEAE-dextran mediated transfection, liposome-mediated transfection, conjugation, transduction, and the like. For stable transformation, a nucleic acid can further include a selectable marker. Suitable selectable markers include antibiotic resistance genes that confer, for example, resistance to phleomycin, nourseothricin, G418, hygromycin B, neomycin, tetracycline, chloramphenicol, or kanamycin, genes that complement auxotrophic deficiencies, and the like. (See below for more detail.)

Various embodiments of the invention use an expression vector that includes a recombinant nucleic acid encoding a protein involved in a metabolic or biosynthetic pathway. Suitable expression vectors include, but are not limited to viral vectors, phage vectors, bacteriophage vectors, plasmids, phagemids, cosmids, fosmids, bacterial artificial chromosomes, P1-based artificial chromosomes, yeast plasmids, yeast artificial chromosomes, and any other vectors specific for cells of interest.

Useful vectors can include one or more selectable marker genes to provide a phenotypic trait for selection of transformed cells. The selectable marker gene encodes a protein necessary for the survival or growth of transformed cells grown in a selective culture medium. Cells not transformed with the vector containing the selectable marker gene will not survive in the culture medium. Typical selection genes encode proteins that (a) confer resistance to antibiotics or other toxins, e.g., nourseothricin, G418, hygromycin B, ampicillin, neomycin, methotrexate, or tetracycline, (b) complement auxotrophic deficiencies, or (c) supply critical nutrients not available from complex media. In alternative embodiments, the selectable marker gene is one that encodes orotidine 5′-phosphate decarboxylase, dihydrofolate reductase or confers neomycin resistance (for use in eukaryotic cell culture).

The coding sequence in the expression vector is operably linked to an appropriate expression control sequence (promoters, enhancers, and the like) to direct synthesis of the encoded gene product. Such promoters can be derived from endogenous or exogenous sources. Thus, the recombinant genes of the invention can comprise a coding sequence operably linked to a heterologous genetic element, such as a promoter, enhancer, ribosome binding site, etc. “Heterologous” in this context refers to a genetic element that is not operably linked to the coding sequence in nature. Depending on the cell/vector system utilized, any of a number of suitable transcription and translation control elements, including constitutive and inducible promoters, transcription enhancer elements, transcription terminators, etc. can be used in the expression vector (see e.g., Bitter et al. (1987) Methods in Enzymology, 153:516-544).

Non-limiting examples of suitable promoters for use within a eukaryotic cell are typically viral in origin and include the promoter of the mouse metallothionein I gene (Hamer et al. (1982) J. Mol. Appl. Gen. 1:273); the TK promoter of Herpes virus (Mcknight (1982) Cell 31:355); the SV40 early promoter (Benoist et al. (1981) Nature (London) 290:304); the Rous sarcoma virus promoter; the cytomegalovirus promoter (Foecking et al. (1980) Gene 45:101); and the yeast gal4 gene promoter (Johnston et al. (1982) PNAS (USA) 79:6971; Silver et al. (1984) PNAS (USA) 81:5951.

Coding sequences can be operably linked to an inducible promoter. Inducible promoters are those wherein addition of an effector induces expression. Suitable effectors include proteins, metabolites, chemicals, or culture conditions capable of inducing expression. Alternatively, a coding sequence can be operably linked to a repressible promoter. Repressible promoters are those wherein addition of an effector represses expression.

In some versions, the cell is genetically modified with a recombinant nucleic acid encoding a biosynthetic pathway gene product that is operably linked to a constitutive promoter. Suitable constitutive promoters are known in the art.

Nucleic acids encoding proteins desired to be expressed in a cell may be codon-optimized for that particular type of cell. Codon optimization can be performed for any nucleic acid by “OPTIMUMGENE”-brand gene design system by GenScript (Piscataway, N.J.).

Methods for transforming yeast cells with recombinant DNA and producing polypeptides therefrom are disclosed by Clontech Laboratories, Inc., Palo Alto, Calif., USA (in the product protocol for the “YEASTMAKER”-brand yeast transformation system kit); Reeves et al. (1992) FEMS Microbiology Letters 99:193-198; Manivasakam and Schiestl (1993) Nucleic Acids Research 21(18): 4414-5; and Ganeva et al. (1994) FEMS Microbiology Letters 121:159-64. Expression and transformation vectors for transformation into many yeast strains are available. For example, expression vectors have been developed for the following yeasts: Candida albicans (Kurtz, et al. (1986) Mol. Cell. Biol. 6:142); Candida maltosa (Kunze et al. (1985) J. Basic Microbiol. 25:141); Hansenula polymorpha (Gleeson et al. (1986) J. Gen. Microbiol. 132:3459) and Roggenkamp et al. (1986) Mol. Gen. Genet. 202:302); Kluyveromyces fragilis (Das et al. (1984) J. Bacteriol. 158:1165), Kluyveromyces lactis (De Louvencourt et al. (1983). J. Bacteriol. 154:737) and Van den Berg et al. (1990) Bio/Technology 8:135); Pichia quillerimondii (Kunze et al. (1985) J. Basic Microbiol. 25:141); Pichia pastoris (Cregg et al. (1985) Mol. Cell. Biol. 5:3376; U.S. Pat. Nos. 4,837,148; and 4,929,555); Saccharomyces cerevisiae (Hinnen et al. (1978) Proc. Natl. Acad. Sci. USA 75:1929 and Ito et al. (1983) J. Bacteriol. 153:163); Schizosaccharomyces pombe (Beach et al. (1981) Nature 300:706); and Yarrowia lipolytica (Davidow et al. (1985) Curr. Genet. 10:380-471 and Gaillardin et al. (1985) Curr. Genet. 10:49), Genetic transformation systems for metabolic engineering have been developed specifically for a number of lipogenic yeasts including Mucor circinelloides (Zhang et al. (2007) Microbiology—Sgm 153, 2013-2025), Yarrowia lipolytica (Xuan et al. (1988) Current Genetics 14, 15-21), Rhodotorula glutinis (Li et al. (2012) Appl Microbiol Biotechnol 97(11): 4927-36), Rhodosporidium toruloides (Zhu et al. (2012) Nature Communications, Vol. 3), Lipomyces starkeyi (Calvey et al. (2014) Current Genetics 60, 223-23; Oguro et al. (2015) Bioscience Biotechnology and Biochemistry 79, 512-515), and Trichosporon oleaginosus (Gomer et al. (2016) Green Chemistry 18, 2037-2046).

An aspect of the invention includes methods of processing organic substrates with the recombinant yeasts of the invention.

The methods involve consuming certain organics while producing other organics. As used herein, “organic” refers to any organic compound, molecule, or polymer capable of being consumed or produced by a microorganism. Exemplary organics include but are not limited to carbohydrates (simple sugars, oligosaccharides, polysaccharides), nucleotides, nucleosides, nucleic acids, polypeptides, organic acids (including amino acids), and organic compounds. Specific examples of organics include cellulose, hemicellulose (e.g., xylan, arabinan, etc.) glucan, glucose, xylose, arabinose, lactic acid, glycerol, acetic acid, butanediol, ethanol, fatty acids, acylglycerols, enzymes (amylases, glucosidases, etc.), among others. As used herein, “consume” refers to the reduction of a certain component from a medium and can encompass direct uptake of the component for internal metabolic processing thereof or external processing of the component optionally followed by uptake of resulting products for internal metabolic processing. The consumption of cellulose and hemicellulose with the recombinant yeasts of the invention, for example, may comprise breaking down the cellulose and hemicellulose with secreted enzymes heterologously introduced in the yeasts, taking up the byproducts thereof, and metabolizing the byproducts to produce organics such as lipid.

The recombinant yeasts of the invention are particularly effective at consuming certain organics that other microorganisms either cannot consume or cannot do so effectively. These organics include cellulose, hemicellulose, glycerol, cellobiose, xylose, lactic acid, trehalose, and oligosaccharides. Accordingly, an aspect of the methods of the invention includes the consumption of these and other organics from the medium. In certain versions of the invention, contacting the medium with the yeast reduces an amount of any one or more of these or other organics to less than about 80% w/w, less than about 70% w/w, less than about 60% w/w. less than about 50% w/w, less than about 45% w/w, less than about 40% w/w, less than about 35% w/w, less than about 30% w/w, less than about 25% w/w, less than about 20% w/w, less than about 15% w/w, less than about 10% w/w, less than about 5% w/w, less than about 2.5% w/w, or less than about 1% w/w of the initial amount.

In some aspects of the invention, the medium comprises one or more components selected from cellulose, xylan, arabinan, glucan, glucose, trehalose, xylose, arabinose, lactic acid, glycerol, acetic acid, butanediol, and ethanol. The medium, for example, may comprise cellulose in an amount of from about 0.05 g/L to about 500 g/L, from about 0.5 g/L to about 50 g/L. or about 5 g/L: hemicellulose in an amount of from about 0.05 g/L to about 500 g/L, from about 0.5 g/L to about 50 g/L, or about 5 g/L; xylan in an amount of from about 0.05 g/L to about 500 g/L, from 0.5 g/L to about 50 g/L, or about 5 g/L; arabinan in an amount of from about 0.005 g/L to about 50 g/L, from about 0.05 g/L to about 5 g/L, or about 0.5 g/L; glucan in an amount of from about 0.1 g/L to about 1000 g/L, from about 1 g/L to about 100 g/L, or about 10 g/L; glucose in an amount of from about 0.01 to about 100 g/L, from about 0.1 g/L to about 10 g/L, or about 1 g/L; trehalose in an amount of from about 0.01 to about 100 g/L or from about 0.1 g/L to about 10 g/L; xylose in an amount of from about 0.01 g/L to about 100 g/L, from about 0.1 g/L to about 10 g/L, or about 1 g/L; arabinose in an amount of from about 0.005 g/L to about 50 g/L; from about 0.05 g/L to about 5 g/L, or about 0.5 g/L; lactic acid in an amount of from about 0.15 g/L to about 1500 g/L, about 1.5 g/L to about 150 g/L, or about 15 g/L; glycerol in an amount of from about 0.15 g/L to about 1500 g/L, from about 1.5 g/L to about 150 g/L, or about 15 g/L; acetic acid in an amount of from about 0.005 g/L to about 50 g/L; from about 0.05 g/L to about 5 g/L, or about 0.5 g/L; butanediol in an amount of from about 0.02 g/L to about 200 g/L, from about 0.2 g/L to about 20 g/L, or about 2 g/L; and/or ethanol in an amount of from about 0.005 g/L to about 50 g/L, from about 0.05 g/L to about 5 g/L, or about 0.5 g/L. Contacting the medium with a yeast may reduce an amount of any one or more of these or other organics to less than about 80%, less than about 70%, less than about 60%, less than about 50%, less than about 45%, less than about 40%, less than about 35%, less than about 30%, less than about 25%, less than about 20%, less than about 15%, less than about 10%, less than about 5%, less than about 2.5%, or less than about 1% of the initial amount.

In certain versions of the invention, the medium comprises a grain ethanol distillation stillage. In certain versions of the invention, the medium comprises a distiller's wet grain from centrifugation of the grain ethanol distillation stillage. In certain version of the invention, the medium comprises a pretreated cellulosic fiber. The cellulosic fiber can be derived from the distiller's wet grain by fractioning the distiller's wet grain into fiber, com oil, and high-protein fractions. Then, the fiber fraction can be carried through a pretreatment by either a multi-stage process that includes alkaline deacetylation and dewatering with a screw press, or hydrolyzed with dilute acid.

In certain versions of the invention, the medium further comprises a thin stillage from grain ethanol distillation stillage. The thin stillage may be made by centrifuging the grain ethanol distillation stillage therefrom, removing oil, concentrating, and filtering, or other thin-stillage processing steps described elsewhere herein or known in the art. The concentrating may comprise evaporating. The thin stillage may be further processed by removing oil and concentrating.

The medium may comprise various amounts of the grain ethanol distillation stillage, the distiller's wet grain, the pretreated cellulosic filter, or the combination of the pretreated cellulosic fiber with the thin stillage. In some versions, the medium may comprise at least about 5% v/v, at least about 15% v/v, at least about 20% v/v, at least about 25% v/v, at least about 30% v/v, at least about 35% v/v, at least about 40% v/v, at least about 45% v/v, at least about 50% v/v, at least about 55% v/v, at least about 60% v/v, at least about 65% v/v, at least about 70% v/v, at least about 75% v/v, at least about 80% v/v, at least about 85% v/v, at least about 90% v/v, at least about 95% v/v, at least about 97% v/v, at least about 99% v/v, or about 100% v/v of grain ethanol distillation stillage, the distiller's wet grain, the pretreated cellulosic filter, or the combination of the pretreated cellulosic fiber with the thin stillage. The grain ethanol distillation stillage, the distiller's wet grain, the pretreated cellulosic filter, or the combination of the pretreated cellulosic fiber with the thin stillage may be diluted with water or other solvents.

The recombinant yeasts of the invention are particularly effective at producing certain organics that other microorganisms either cannot produce or cannot do so effectively. The recombinant yeasts of the invention convert cellulosic and hemicellulosic fibers to corresponding mono- or disaccharides (e.g., glucose, cellobiose, xylose, arabinose, etc.). The produced mono- or disaccharides, together with other components in the medium are utilized to produce organics including, for example, lipids (triacylglycerols, diacylglycerols. monoacylglycerols, fatty acids, etc.). Accordingly, an aspect of the methods of the invention includes the production of these and other organics.

The organic produced by the yeast may be separated or purified from any other component of the spent medium for downstream use in other applications. For example, lipid produced by the yeast may be used for producing biofuels therefrom or used as a replacement for palm or other oils in food applications or other applications.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Summary

The following examples describe tools and methods for making biodiesel precursors from cellulosic materials present in grain ethanol stillage and agricultural harvest residues. Hyper-lipogenic yeast strains (e.g., Lipomyces starkeyi, among others) are engineered to express and secrete carbohydrases such as cellulases, xylanases, and auxiliary enzymes to generate “carbohydrolytic” yeasts capable of consuming currently underutilized ethanol fermentation byproducts and cellulosic residues. The carbohydrolytic yeasts can convert the pretreated waste com fiber or stover into a mixture of fatty acids suitable for biodiesel, a palm oil biosimilar or other metabolic product. This technology enables diversification and increases profitability of the grain ethanol industry while providing a new sustainable source of a palm oil biosimilar.

### Background

Dry-grind grain ethanol constitutes the largest renewable fuel industry in the world, yet it is only marginally profitable. The bioconversion of stillage solubles, fiber, and pretreated com stover into palm oil-like lipids is an area with major commercial potential. The present examples provide for the bioconversion of stillage solubles and fiber, which are low-value byproducts of grain ethanol fermentation, into a high-quality oil suitable for biodiesel, food, cosmetic, chemical, or other uses. This goal has been achieved by metabolic engineering, strain selection, and process development based on metabolically engineered, highly lipogenic L. starkeyi strains that synthesize and secrete enzymes that are appropriate for deconstructing pretreated corn fiber derived from whole stillage.

The present examples show engineered strains that can attain commercially viable rates and yields of lipid on commercial stillage streams, and fermentation conditions that provide cell growth, extracellular enzyme production, and lipid accumulation in bioreactors. The examples provide principal process parameters that determine the optimal rates and yields for lipid production from stillage solubles and pretreated fiber.

Cultivation of wild-type and engineered L. starkeyi can be carried out in batch or fed-batch fermentations to achieve maximal lipid yields. [13-15] Wild-type L. starkeyi normally produces high levels of lipid only at C:N ratios between 40:1 and 60:1. [12] Fermentations using xylose or xylose-glucose mixtures-such as those found in hydrolysates of com stover or other herbaceous materials—can result in higher lipid yields than those solely using glucose. [6, 16, 17] Glycerol is a major component of thin stillage. We selected a platform Z. starkeyi strain (NRRL Y-11557) capable of robust growth on glycerol and glucose-xylose mixtures. This strain is referred to in the following examples as the “wild-type” strain. We engineered wild-type strain to overexpress two diacylglycerol acyltransferases (DGA1, SEQ ID NO: 83; DGA2, SEQ ID NO:85), a malic enzyme (ME, SEQ ID NO:87), and a glycerol-3-phosphate acyltransferase (SCT1, SEQ ID NO:89). The resulting strain is referred to herein as the “XYL403” strain (Table 4). The 403 strain is capable of producing lipids from dissolved organics in thin stillage at relatively low C:N ratios. We further engineered the 403 strain to generate a line of hyper-lipogenic yeasts having an enhanced capacity for glycerol utilization. One of these strains, referred to herein as the “XYL609” strain, is engineered to overexpress a glycerol kinase (GUT1, SEQ ID NO:91), a FAD-dependent glycerol-3-phosphate dehydrogenase (GUT2, SEQ ID NO:93), and a glycerol-3-phosphate dehydrogenase (GPDI, SEQ ID NO:95).

The hyper-lipogenic engineered L. starkeyi XYL403 can yield more than 0.6 pounds of oil per gallon of fermentation broth when ethanol stillage is augmented with a low-cost soluble carbon source such as glucose or starch hydrolysate in fed-batch fermentation processes. This strain of L. starkeyi is able to consume all of the mono- and disaccharides along with glycerol, lactic acid, and acetic acid, but it has little capacity to degrade and consume cellulose or hemicellulose. Nonetheless, the oil produced is similar to palm oil, and can be used for a range of purposes, such as for biodiesel production, and in food and cosmetic applications. However, even in the best of these hyper-lipogenic strains, cellulosic and hemicellulosic polysaccharides are not typically consumed to any significant extent.

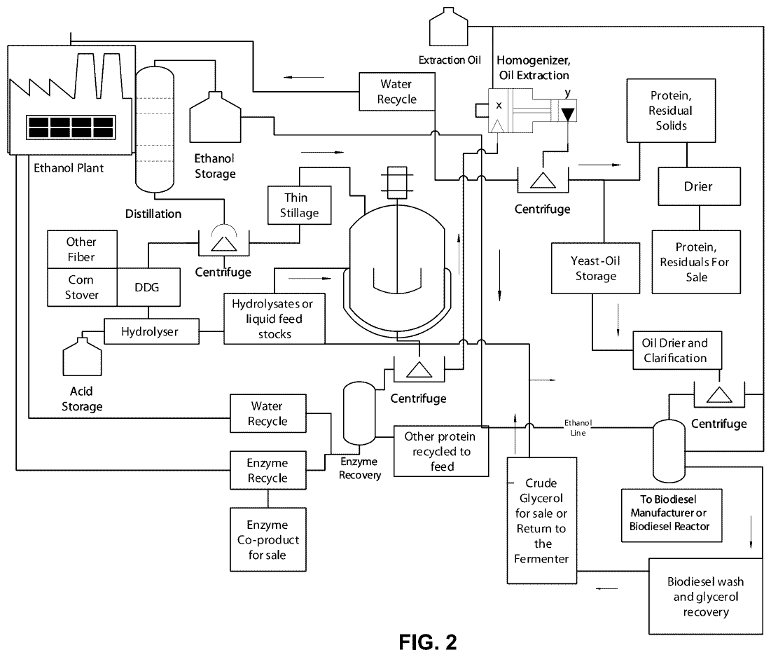

Newer processes under development by the grain processing industry such as the thin stillage solids separation system from ICM, [7] the Fiber By-Pass system by Fluid Quip. [7] the direct hydrolysis of fiber in stillage wet cake by D3Max [8] and a fiber pretreatment system by POET [9] remove and hydrolyze corn fiber either prior to or following saccharification and fermentation. Corn fiber is mainly found in the DDG, the value of which is based largely on the protein rather than the fiber content. [10] By separating fiber from the protein, ethanol producers create a more valuable feed that can be used for livestock outside of ruminants, such as poultry and swine (FIG. 2).

These recently developed commercially available processes separate corn fiber from thin stillage and distiller's wet grain and produce a fiber-free thin stillage concentrate. The fiber-free organics in stillage syrup constitute a highly suitable substrate for lipid production. However, engineering L. starkeyi to secret cellulases, xylanases, and other carbohydrate active enzymes would provide existing mills with the capacity to produce biodiesel precursors from fiber byproducts. Corn fiber from ethanol mills represents a significant collected biomass resource that has few value added uses aside from ruminant feed. [10]

Thin stillage contains sufficient nitrogen to enable L. starkeyi to synthesize cellulases and xylanases that can degrade pretreated cellulosic fiber present in distiller's wet grain. Moreover, this same technology can be used to convert pretreated com stover into energy-dense cellulosic biodiesel. By integrating the bioconversion of waste fermentation byproducts into cellulases and xylanases with the bioconversion of pretreated agricultural residues, it should be possible to greatly increase the production of high energy density oils for biodiesel and other applications. A hydrolytic process has been described that will allow the economic recovery of triglycerides for biodiesel production. [19]

While it is feasible to simply add commercial cellulase and xylanase enzyme cocktails to the stillage medium, consumption of glycerol and lactic acid is repressed by the glucose that is released by cellulose hydrolysis, and the exogenous enzymes are expensive. It is therefore preferable to initiate growth of L. starkeyi on glycerol and free sugars present in the stillage and then add the pretreated cellulosic and hemicellulosic sugars to increase lipid content in the cells.

### Strains and Processes

Previously engineered L. starkeyi strains (e.g., XYL403) can consume essentially all of the soluble organic constituents present in thin stillage, but they cannot consume the insoluble cellulosic and hemicellulosic components, which constitute ≈30 to 40% of stillage organics. The present examples provide engineered carbohydrolytic L. starkeyi strains which are able to consume the insoluble com fiber fraction, thereby greatly increasing the amount of lipid that can be produced from this low-value byproduct. The engineered strains represent consolidated bioprocessing organisms capable of utilizing all of the carbon sources present in thin stillage without the need for expensive enzymatic or thermochemical treatments.

Some of the strains provided herein combine lipogenic engineering with capacity for cellulose and hemicellulose bioconversion to create yeasts that produce high-density, renewable biodiesel and sustainable substitutes for palm oil from domestic resources. This technology will advance the state of the art at dry grind ethanol plants by enabling them to convert organic byproducts currently sold as animal feed into much higher value renewable biofuels. This new commercial path to cellulosic biodiesel from ethanol plants using the genetic augmentations described herein could increase the production of lipid from insoluble com fiber available in ethanol plants today.

### Strains

Because the main carbohydrate content of thin stillage and distiller's wet grain is composed of cellulosic and hemicellulosic sugars, we have expressed in L. starkeyi cellulases, xylanases, arabinoxylanases, and other enzymes that aid in the deconstruction of complex carbohydrate feedstocks in situ. The yeasts have been engineered to secrete enzymes constitutively during their initial growth phase, which allows them to deplete the nitrogen and glycerol in stillage and then consume pretreated fiber as it is added in a lipid accumulation phase. The wild-type and engineered L. starkeyi yeasts produce amylases and amylodextrinases that can be cycled through backset into a liquefaction stage along with the spent broth from clarified thin stillage fermentation. These enzymes can boost liquefaction of starch by loosening the hemicellulosic matrix in which residual starch is bound. By incorporating cellulase and hemicellulase production into lipogenic strains, the cells can accommodate a mild pretreatment process with little or no supplemental enzyme addition. The combination of biomass saccharification and conversion to products in a single step also results in simplified process design and concomitant savings in operational costs. [23]

### Processes

In exemplary processes of the invention, distiller's wet grain from traditional dry-mill centrifugation of whole stillage can be fractionated into fiber, com oil, and high-protein distiller's dry grain fractions. The high-protein distiller's dry grain has a lower fiber content, making it suitable as a feed component for non-ruminants such as poultry, swine, or aquaculture. Conversely, the fiber fraction can be carried through a pretreatment then enzymatically digested. The pretreatment can include either a multi-stage process that includes alkaline deacetylation and dewatering with a screw press, which might be preferred for fiber from a dry fractionation process or hydrolyzed with dilute acid. Yeast oil derived from engineered L. starkeyi capable of converting cellulosic and hemicellulosic sugars into lipid is termed “cellulosic oil”. Cellulosic oil can also be created from using distiller's wet grain or any other com fiber source as a feedstock for the nonconventional lipogenic and cellulosic engineered yeast strains. The dissolved organics can be recovered from the thin stillage through a separate fractionation step and then concentrated through triple-effect evaporation to make clarified thin stillage (CTS) concentrate.

The technology for clarified thin stillage fermentation can be combined with pretreated cellulose, hemicellulose, and residual starch from fractionated distiller's wet grain. By so doing, cellulosic oil can be produced from the stillage. The fermentation of clarified thin stillage has been demonstrated in batch, [12] fed-batch [13, 14] and continuous culture [20] using stillage-based or model media at bench-scale in 3- to 7-L reactors. Typically, fed-batch fermentations show the highest yields, so fed-batch fermentations can be targeted to create hyper-lipogenic cells from the engineered strains described herein. With a fed-batch fermentation, L. starkeyi initially takes up any available glucose before consuming xylose, glycerol, amylodextrins, arabinose, and lactic and acetic acids. Lipid production can be optimized using a 2:1 glucose:xylose mixture, [21] and this synthetic medium can be adapted to continuous and fed-batch cultivation. A 2:1:1 glucose:xylose:arabinose mixture approximates the sugar ratios obtained from hydrolysates of com fiber.

### Examples from Experimental Findings

### Verification Trials

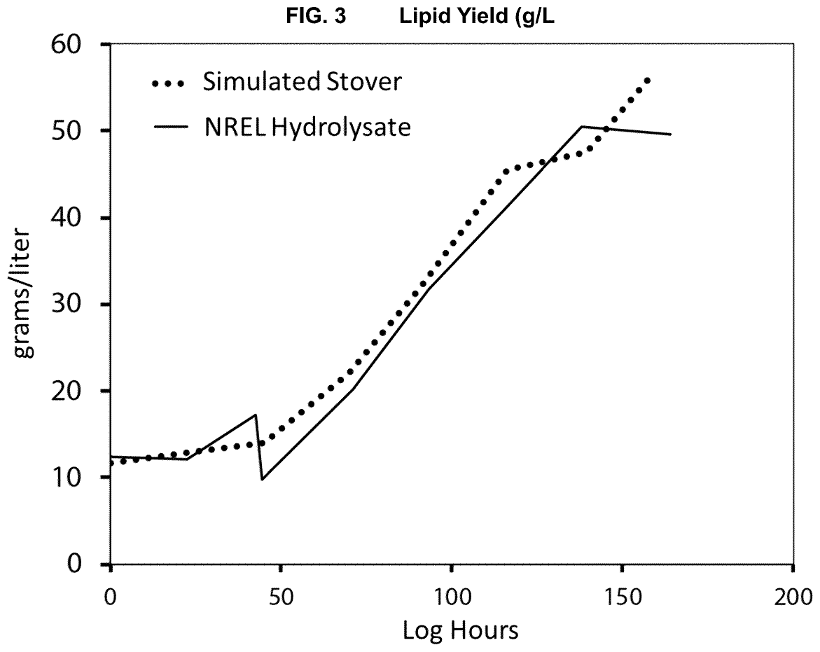

Our lipogenic technology was initial tested using the XYL403 strain with clarified stillage as a nitrogen source and either an enzymatic hydrolysate of com stover, or a suspension that contained just the sugar components of the com stover hydrolysate (glucose and xylose), as carbon sources. The results of this experiment are shown in FIG. 3. In both instances, the cultures grew well and began accumulating lipid after 48 hours, with the final lipid content being approximately 50 g/L. These data demonstrated that an enzymatic digest of pretreated com stover hydrolysate enables cell growth and lipid accumulation by XYL403 and the hydrolysate utilized equally as well as a pure sugar suspension of glucose and xylose, suggesting this and other corn derived substrates could be used for growing XYL403 and other Lipomyces starkeyi strains that have been augmented with cellulases, hemicelluloses, and different carbohydrate active enzymes.

We also conducted shake flask trials on various concentrations of Deacetylated Mechanically Refined (DMR) com stover received from National Renewable Energy Laboratory (NREL) using the wild-type NRRL Y-11557 strain without addition of hydrolysate or free sugar. This material contains small amounts of lactose, xylose, arabinose, and xylitol which L. starkeyi quickly utilized within the first 24 hours to support cell growth. However, the remaining components (mostly fiber) were not consumed, and we were unable to obtain significant cell growth and lipid production beyond the first 24 hours. Importantly, no toxicity was observed in cells grown in the presence of the NREL material, although at concentrations 30% or higher the suspension was too viscous for agitation in a shake flask. Strains augmented with carbohydrate active enzymes should perform significantly better on this material, as the fiber represents a rich untapped carbon source and should decrease in viscosity as it is deconstructed and utilized.

### Identification of Secretion Signals

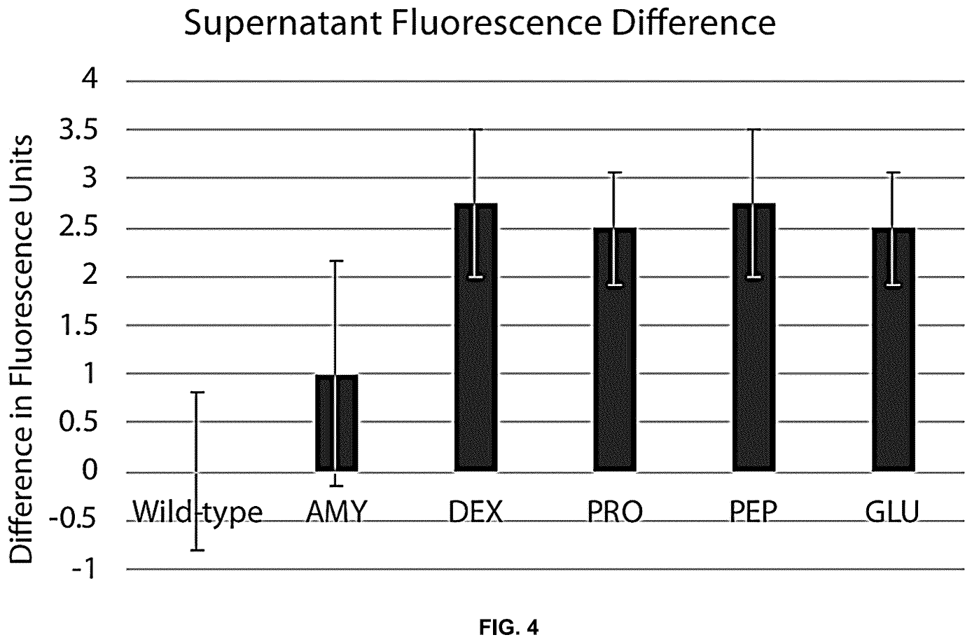

Engineering a Lipomyces starkeyi strain capable of deconstructing and utilizing the sugars in corn fiber are both synthesized and secreted in order to act on the fiber substrate. We identified functional secretion signals for expression in Lipomyces starkeyi by fusing leader sequences derived from both native and heterologous proteins onto a reporter protein (GFP) and monitoring the culture for secretion of the reporter protein into the media. The five secretion signals evaluated were derived from an α-amylase (AMY), a dextranase (DEX), an alkaline extracellular protease (PRO), a subtilisin like peptidase (PEP), and an α-glucosidase (GLU) (Table 3). The length of the leader sequence to fuse to GFP was determined using an algorithm, our knowledge of protein secretion in yeast, and conservative thresholds. [31,32] The supernatant fluorescence in the GFP secreting transformant cultures was higher than the wild-type cultures (FIG. 4), indicating that all five of these secretion signals are functional in Lipomyces starkeyi. These secretion signals can be used with various cellulases, hemicellulases, and other carbohydrate active enzymes for secretion into the culture medium.

### Identification of Gene Targets

Several carbohydrate active enzyme genes from different species were chosen for overexpression, secretion, and activity analyses in Lipomyces starkeyi. Genes from pathogenic organisms were avoided but not entirely excluded. All genes were codon-optimized by using a proprietary algorithm based on the codon preferences of highly expressed genes in Lipomyces starkeyi, and manually adjusted when necessary. The synthetic DNA was constructed by a company specialized in DNA synthesis, and the double stranded DNA fragment was used in cloning (typically Gibson isothermal) assemblies. A summary of the genes investigated, their species of origin, and glycoside hydrolase (GH) family classification, is provided in Table 2. Other information pertaining to each cassette as cloned is also provided, such as the secretion signal utilized, the resistance marker used for selection, and the promoter/terminator pairing for expression. Sequence ID NOs for elements used in this invention are summarized in Table 3.

TABLE 2
Summary of cassettes.

| | | GH | Secretion | Resistance | Promoter/ |
|---|---|---|---|---|---|
| Enzyme (Abbreviation) | Organism | Family | Signal | Marker | Terminator |
| Exo-cellobiohydrolase I | Trichoderma reesei | 7 | PRO | BLE | ACT1p/ACT1t |
| (TrCBH1) | | | | | |
| Exo-cellobiohydrolase I | Chimeric enzyme | 7 | PRO | BLE | ACT1p/ACT1t |
| (TeCD/TrCBM) | (Trichoderma reesei | | | | |
| (TeTrCBHI) | and Talaromyces | | | | |
| | emersonii) | | | | |
| Exo-cellobiohydrolase I | Aspergillus niger | 7 | PRO | BLE | ACT1p/ACT1t |
| (AnCBHB) | | | | | |
| Exo-cellobiohydrolase I | Trichoderma reesei | 6 | DEX | HYG | CYC1p/PDA1t |
| (TrCBH2) | | | | | |
| Exo-cellobiohydrolase II | Aspergillus flavus | 6 | DEX | HYG | CYC1p/PDA1t |
| (AfCBH2) | | | | | |
| Endoglucanase (AnEglA) | Aspergillus niger | 12 | PEP | NAT | PDA1p/PDE3t |
| Endoglucanase (AnEglB) | Aspergillus niger | 5 | PEP | NAT | PDA1p/PDE3t |
| Endoglucanase | Aureobasidium | 5 | PEP | NAT | PDA1p/PDE3t |
| (ApCel5A) | pulluleas | | | | |
| Endoglucanase (TrEGII) | Trichoderma reesei | 5 | PEP | NAT | PDA1p/PDE3t |
| Endoxylanase | Aspergillus niger | 10 | DEX | HYG | ACO1p/PDE3t |
| (AnXYNA) | | | | | |
| Endoxylanase (ThXYN2) | Trichoderma | 11 | DEX | HYG | ACO1p/PDE3t |
| | harzianum | | | | |
| Endoxylanase | Fusarium | 10 | DEX | HYG | ACO1p/PDE3t |
| (FgXYNC) | graminearum | | | | |
| Endoxylanase (TfXYNB) | Talaromyces | 11 | DEX | HYG | ACO1p/PDE3t |
| | funiculosus | | | | |
| Endoxylanase (TfXYND) | Talaromyces | 10 | DEX | HYG | ACO1p/PDE3t |
| | funiculosus | | | | |
| Lytic Polysaccharide | Trichoderma reesei | AA9 | PRO | NAT | PDE3p/PDA1t |
| Monooxygenase | | | | | |
| (LPMO) (TrCEL61A) | | | | | |
| Lytic Polysaccharide | Trichoderma reesei | AA9 | PRO | NAT | PDE3p/PDA1t |
| Monooxygenase | | | | | |
| (LPMO) (TrCEL61B) | | | | | |
| α-L-Arabinofuranosidase | Aspergillus niger | 62 | GH3.1 | ** | *** |
| (AxhA) | | | | | |
| α-L-Arabinofuranosidase | Aspergillus phoenicis | 54 | GH3.1 | ** | *** |
| B (AbfB) | | | | | |
| α-L-Arabinofuranosidase | Aspergillus niger | 54 | GH3.1 | ** | *** |
| B (AbfB) | | | | | |
| α-L-Arabinofuranosidase | Thermobacillus | 51 | * | ** | *** |
| | xylanilyticus | | | | |
| Xylanase B (TcXyn30B) | Talaromyces | 30 | * | ** | *** |
| | cellulolyticus | | | | |
| Xylanase C (TcXyn30C) | Talaromyces | 30 | * | ** | *** |
| | cellulolyticus | | | | |
| Glucuronoarabinoxylan | Ruminiclostridium | 30 | * | ** | *** |
| endo-1,4-beta-xylanase | papyrosolvens | | | | |
| (RpGluAraXyl) | | | | | |
| Acetylxylan esterase A | Aspergillus niger | NA | * | ** | *** |
| (AxeA) | | | | | |

“*” indicates any above listed secretion signal;
“**” indicates any above listed resistance marker;
“***” indicates any above listed promoter/terminator.

TABLE 3
Elements used in the invention.

| | SEQ ID NOs | |
|---|---|---|
| | DNA | Protein |
| Cellulolytic Enzymes | | |
| Exo-cellobiohydrolase I | Positions 55-1545 of SEQ ID NO: 1 | Positions 19-514 of SEQ |
| (TrCBH1) | | ID NO: 2 |
| Exo-cellobiohydrolase I | Positions 55-1551 of SEQ ID NO: 3 | Positions 19-516 of SEQ |
| (TeCD/TrCBM) | | ID NO: 4 |
| Exo-cellobiohydrolase I | Positions 55-1593 of SEQ ID NO: 5 | Positions 19-530 of SEQ |
| (AnCBHB) | | ID NO: 6 |
| Exo-cellobiohydrolase II | Positions 73-1416 of SEQ ID NO: 7 | Positions 25-471 of SEQ |
| (TrCBH2) | | ID NO: 8 |
| Exo-cellobiohydrolase II | Positions 73-1278 of SEQ ID NO: 9 | Positions 25-425 of SEQ |
| (AfCBH2) | | ID NO: 10 |
| Endoglucanase (AnEglA) | Positions 94-764 of SEQ ID NO: 11 | Positions 32-254 of SEQ |
| | | ID NO: 12 |
| Endoglucanase (AnEglB) | Positions 94-1033 of SEQ ID | Positions 32-344 of SEQ |
| | NO: 13 | ID NO: 14 |
| Endoglucanase (ApCel5A) | Positions 94-1324 of SEQ ID | Positions 32-441 of SEQ |
| | NO: 15 | ID NO: 16 |
| Endoglucanase (TrEGII) | Positions 94-1285 of SEQ ID | Positions 32-428 of SEQ |
| | NO: 17 | ID NO: 18 |
| Endoxylanase (AnXYNA) | Positions 73-990 of SEQ ID NO: 19 | Positions 25-329 of SEQ |
| | | ID NO: 20 |
| Endoxylanase (ThXYN2) | Positions 73-681 of SEQ ID NO: 21 | Positions 25-226 of SEQ |
| | | ID NO: 22 |
| Endoxylanase (FgXYNC) | Positions 73-999 of SEQ ID NO: 23 | Positions 25-332 of SEQ |
| | | ID NO: 24 |
| Endoxylanase (TfXYNB) | Positions 73-804 of SEQ ID NO: 25 | Positions 25-267 of SEQ |
| | | ID NO: 26 |
| Endoxylanase (TfXYND) | Positions 73-1239 of SEQ ID | Positions 25-412 of SEQ |
| | NO: 27 | ID NO: 28 |
| Lytic Polysaccharide | Positions 55-744 of SEQ ID NO: 29 | Positions 19-247 of SEQ |
| Monooxygenase (LPMO) | | ID NO: 30 |
| (TrCEL61A) | | |
| Lytic Polysaccharide | Positions 55-1023 of SEQ ID | Positions 19-340 of SEQ |
| Monooxygenase (LPMO) | NO: 31 | ID NO: 32 |
| (TrCEL61B) | | |
| α-L-Arabinofuranosidase | Positions 79-999 of SEQ ID | Positions 27-332 of SEQ |
| (AnAxhA) | NOs: 33-34 and 96; Positions 58- | ID NO: 35; Positions 20- |
| | 978 of SEQ ID NO: 98 | 325 of SEQ ID NO: 97 |
| α-L-Arabinofuranosidase | Positions 55-1500 of SEQ ID | Positions 19-499 of SEQ |
| B (AbfB) (Aspergillus | NOs: 36-37 and 99; Positions 58- | ID NO: 38; Positions 20- |
| phoenicis) | 1503 of SEQ ID NO: 101 | 500 of SEQ ID NO: 100 |
| α-L-Arabinofuranosidase | Positions 55-1500 of SEQ ID | Positions 19-499 of SEQ |
| B (AbfB) (Aspergillus | NO: 39; Positions 58-1503 of SEQ | ID NO: 40; Positions 20- |
| niger) | ID NO: 102 | 500 of SEQ ID NO: 103 |
| α-L-Arabinofuranosidase | SEQ ID NO: 41 | SEQ ID NO: 42 |
| (Thermobacillus | | |
| xylanilyticus) | | |
| Xylanase B (TcXyn30B) | SEQ ID NO: 43 | SEQ ID NO: 44 |
| chain A | | |
| Xylanase B (TcXyn30B) | SEQ ID NO: 45; 111 | SEQ ID NO: 46 |
| chain B | | |
| Xylanase C (TcXyn30C) | SEQ ID NO: 47 | SEQ ID NO: 48 |
| chain A | | |
| Xylanase C (TcXyn30C) | SEQ ID NO: 49; 112 | SEQ ID NO: 50 |
| chain B | | |
| Glucuronoarabinoxylan | SEQ ID NO: 51; 113 | SEQ ID NO: 52 |
| endo-1,4-beta-xylanase | | |
| Acetylxylan esterase A | SEQ ID NO: 53 | SEQ ID NO: 54 |
| (AxeA) | | |
| Secretion Signal | | |
| PRO | SEQ ID NO: 55 | SEQ ID NO: 56 |
| DEX | SEQ ID NOs: 57-60 | SEQ ID NO: 61 |
| PEP | SEQ ID NO: 62 | SEQ ID NO: 63 |
| AMY | SEQ ID NO: 64 | SEQ ID NO: 65 |
| GLU | SEQ ID NO: 66 | SEQ ID NO: 67 |
| GH3.1 | SEQ ID NO: 104 | SEQ ID NO: 105 |
| AxhA | SEQ ID NO: 106 | SEQ ID NO: 107 |
| AbfB | SEQ ID NO: 108 | SEQ ID NO: 109 |
| Lipomyces starkeyi NRRL | SEQ ID NO: 114 | SEQ ID NO: 115 |
| Y11557 Protein ID 61256 | | |
| Lipomyces starkeyi NRRL | SEQ ID NO: 116 | SEQ ID NO: 117 |
| Y11557 Protein ID | | |
| 322386 | | |
| Lipomyces starkeyi NRRL | SEQ ID NO: 118 | SEQ ID NO: 119 |
| Y11557 Protein ID 67146 | | |
| Lipomyces starkeyi NRRL | SEQ ID NO: 120 | SEQ ID NO: 121 |
| Y11557 Protein ID 7314 | | |
| Lipomyces starkeyi NRRL | SEQ ID NO: 122 | SEQ ID NO: 123 |
| Y11557 Protein ID 5250 | | |
| Lipomyces starkeyi NRRL | SEQ ID NO: 124 | SEQ ID NO: 125 |
| Y11557 Protein ID 74374 | | |
| Lipomyces starkeyi NRRL | SEQ ID NO: 126 | SEQ ID NO: 127 |
| Y11557 Protein ID 75319 | | |
| Lipomyces starkeyi NRRL | SEQ ID NO: 128 | SEQ ID NO: 129 |
| Y11557 Protein ID 6714 | | |
| Lipomyces starkeyi NRRL | SEQ ID NO: 130 | SEQ ID NO: 131 |
| Y11557 Protein ID | | |
| 338499 | | |
| Lipomyces starkeyi NRRL | SEQ ID NO: 132 | SEQ ID NO: 133 |
| Y11557 Protein ID | | |
| 338374 | | |
| Lipomyces starkeyi NRRL | SEQ ID NO: 134 | SEQ ID NO: 135 |
| Y11557 Protein ID 5459 | | |
| Lipomyces starkeyi NRRL | SEQ ID NO: 136 | SEQ ID NO: 137 |
| Y11557 Protein ID 962 | | |
| Lipomyces starkeyi NRRL | SEQ ID NO: 138 | SEQ ID NO: 139 |
| Y11557 Protein ID 235 | | |
| Lipomyces starkeyi NRRL | SEQ ID NO: 140 | SEQ ID NO: 141 |
| Y11557 Protein ID 49224 | | |
| Lipomyces starkeyi NRRL | SEQ ID NO: 142 | SEQ ID NO: 143 |
| Y11557 Protein ID 76563 | | |
| Lipomyces starkeyi NRRL | SEQ ID NO: 144 | SEQ ID NO: 145 |
| Y11557 Protein ID 6909 | | |
| Lipomyces starkeyi NRRL | SEQ ID NO: 146 | SEQ ID NO: 147 |
| Y11557 Protein ID 61256 | | |
| Lipomyces starkeyi NRRL | SEQ ID NO: 148 | SEQ ID NO: 149 |
| Y11557 Protein ID 84172 | | |
| Lipomyces starkeyi NRRL | SEQ ID NO: 150 | SEQ ID NO: 151 |
| Y11557 Protein ID | | |
| 120665 | | |
| Lipomyces starkeyi NRRL | SEQ ID NO: 152 | SEQ ID NO: 153 |
| Y11557 Protein ID 75368 | | |
| Lipomyces starkeyi NRRL | SEQ ID NO: 154 | SEQ ID NO: 155 |
| Y11557 Protein ID | | |
| 111843 | | |
| Lipomyces starkeyi NRRL | SEQ ID NO: 156 | SEQ ID NO: 157 |
| Y11557 Protein ID 71087 | | |
| Lipomyces starkeyi NRRL | SEQ ID NO: 158 | SEQ ID NO: 159 |
| Y11557 Protein ID 72441 | | |
| Lipomyces starkeyi NRRL | SEQ ID NO: 160 | SEQ ID NO: 161 |
| Y11557 Protein ID 55318 | | |
| Lipomyces starkeyi NRRL | SEQ ID NO: 162 | SEQ ID NO: 163 |
| Y11557 Protein ID 1422 | | |
| Lipomyces starkeyi NRRL | SEQ ID NO: 164 | SEQ ID NO: 165 |
| Y11557 Protein ID 66955 | | |
| Promoter | | |
| ACT1 | SEQ ID NO: 68 | — |
| CYC1 | SEQ ID NO: 69 | — |
| PDA1 | SEQ ID NO: 70 | — |
| ACO1 | SEQ ID NO: 71 | — |
| PDE3 | SEQ ID NO: 72 | — |
| Histone bi-directional | SEQ ID NO: 110 | — |
| promoter | | |
| Terminator | | |
| ACT1 | SEQ ID NO: 73 | — |
| PDA1 | SEQ ID NO: 74 | — |
| PDE3 | SEQ ID NO: 75 | — |
| Resistance Marker | | |
| BLE | SEQ ID NO: 76 | SEQ ID NO: 77 |
| HYG | SEQ ID NO: 78 | SEQ ID NO: 79 |
| NAT | SEQ ID NO: 80 | SEQ ID NO: 81 |
| Lipogenic Enzymes | | |
| Diacylglycerol | SEQ ID NO: 82 | SEQ ID NO: 83 |
| Acyltransferase (DGA1) | | |
| Secondary Diacylglycerol | SEQ ID NO: 84 | SEQ ID NO: 85 |
| Acyltransferase (DGA2) | | |
| Malic Enzyme (ME) | SEQ ID NO: 86 | SEQ ID NO: 87 |
| Glycerol-3-Phosphate | SEQ ID NO: 88 | SEQ ID NO: 89 |
| Acyltransferase (SCT1) | | |
| Glycerol Kinase (GUT1) | SEQ ID NO: 90 | SEQ ID NO: 91 |
| Glycerol-3-Phosphate | SEQ ID NO: 92 | SEQ ID NO: 93 |
| Dehydrogenase | | |
| (GPD2/GUT2) | | |
| Glycerol-3-Phosphate | SEQ ID NO: 94 | SEQ ID NO: 95 |
| Dehydrogenase (GPD1) | | |

TABLE 4
Exemplary strains.

| Strain | Background | Added Genes |
|---|---|---|
| CEL3005 | L. starkeyi NRRL Y-11557 | TeCD/TrCBM, TrCBHII, |
| | | AnEglA |
| CEL3006 | L. starkeyi NRRL Y-11557 | TeCD/TrCBM, TrCBHII, |
| | | AnEglB |
| CEL3007 | L. starkeyi NRRL Y-11557 | TeCD/TrCBM, TrCBHII, |
| | | ApCel5A |
| CEL3008 | L. starkeyi NRRL Y-11557 | TeCD/TrCBM, TrCBHII, |
| | | TrEGII |
| CEL3022 | L. starkeyi NRRL Y-11557 | TeCD/TrCBM, ApCel5A × |
| | | TrCBHII |
| CEL4001 | L. starkeyi NRRL Y-11557 | TeCD/TrCBM,TrCBHII, |
| | | TrEGII, ThXYN2 |
| CEL4002 | L. starkeyi NRRL Y-11557 | TeCD/TrCBM, TrCBHII, |
| | | TrEGII, ThXYN2 |
| CEL5001 | L. starkeyi NRRL Y-11557 | TeCD/TrCBM, TrCBHII, |
| | | TrEGII, ThXYN2, TrCEL61B, |
| | | LPMO (OK) |
| XYL403 | L. starkeyi NRRL Y-11557 | DGA1, DGA2, ME, SCT1 |
| CEL3427 | XYL403 | TrCBHII, TrEGII |
| CEL3486 | XYL403 | TeCD/TrCBM, TrEGII, |
| | | TrCBHII |
| CEL4408 | XYL403 | TeCD/TrCBM, TrCBHII, |
| | | TrEGII TharXYN2 |
| CEL5401 | XYL403 | TeCD/TrCBM, TrCBHII, |
| | | TrEGII, ThXYN2, AnAxhA |

### Establishing Screening Assays for Single Transformants

Lipomyces starkeyi has a strong preference to integrate linear DNA fragments into its genome by random, non-homologous end joining (NHEJ) repair mechanisms rather than by site-specific homologous recombination. The random nature of NHEJ integration potentially can result in a multitude of unique transformants from a single expression cassette. The sites at which integration occurs can greatly affect gene expression, so generating a large population of randomly integrated transformants is a powerful and dominant feature in strain development efforts. Random integration followed by high-throughput screening can lead to very high expression levels for individual integrated enzymes.

An exemplary strategy for obtaining strains exhibiting high levels of expression and secretion of multiple enzymes—i.e., consolidated expression—is as follows: (1) Transform an individual enzyme into a starting strain (host) using a loxP-flanked selection marker specific to the enzyme being introduced. (2) Screen hundreds of transformed host strains for their extracellular expression of the individual enzyme. (3) Identify the transformed host that best expresses each individual enzyme. (4) Starting with the same starting strain (host), repeat steps 1 through 3 with a second enzyme and a second loxP-flanked selection marker, and (5) use the separate selection markers with the separate enzymes to identify mated strains carrying both highly expressed enzymes. (6) Using a third lox-P-flanked selection marker, introduce a third highly expressed enzyme, and repeat steps 1 through 5. (7) Use a fourth selection marker to introduce Cre, which excises the first three selection markers from the host strain carrying the combined, consolidated, highly expressed enzymes. Because the starting host genomic background for the transformant lines are the same, the genetic background of the consolidated strain should remain the same. It is possible that deletions, duplications or rearrangements may occur during the transformation, selection and mating process, so a final step of screening consolidated hyper-secreting strains for maximal activity is preferred. Such screens may be carried out on individual consolidated strains or through competitive growth or evolutionary adaptation on hydrolysates or pretreated substrates. It should be apparent that the newly-created consolidated host, which has been cured of selection markers can be used as a starting strain for introducing the next set of enzymes.

### Exo-Cellobiohydrolases

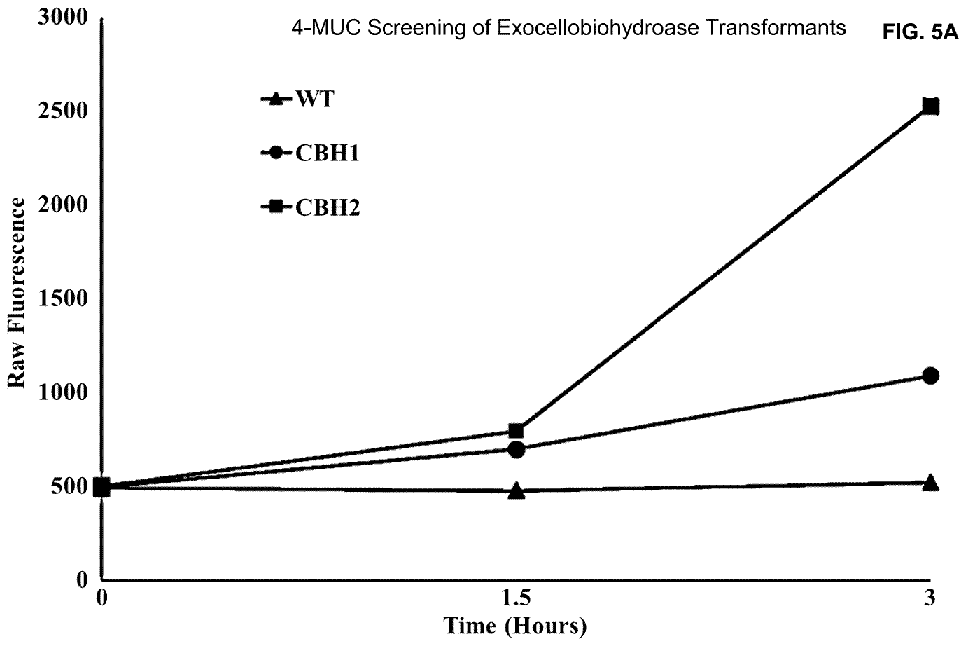

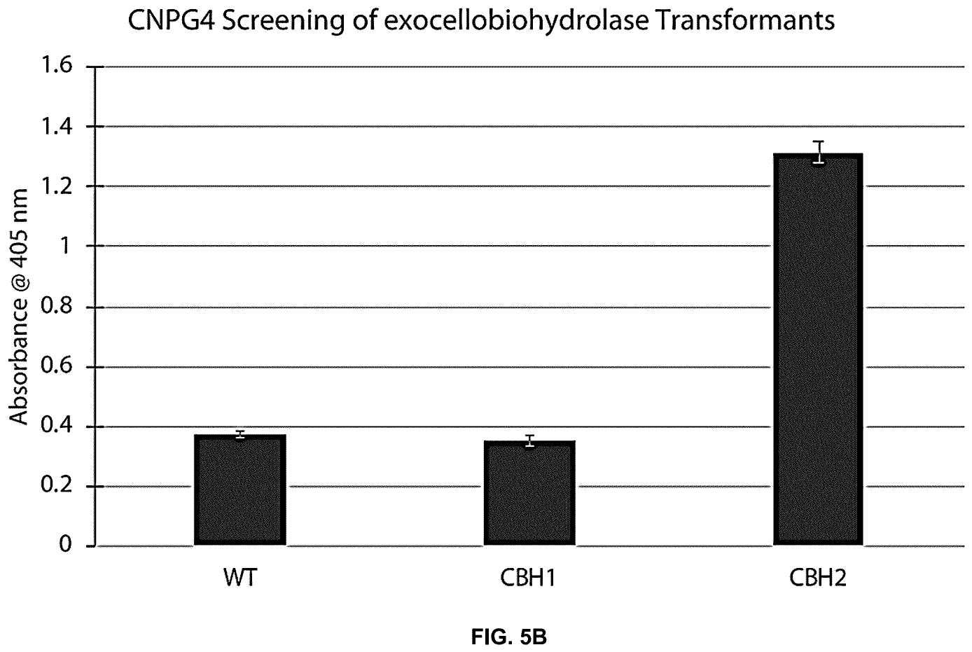

Exo-cellobiohydrolases were screened for activity. Exo-cellobiohydrolases fall into one of two classes, depending on which end of an oligosaccharide they act upon. Those cleaving and releasing cellobiose from the reducing end of the sugar chain are categorized within the cellobiohydrolase I (CBH1) class, whereas those cleaving and releasing cellobiose from the non-reducing end of the sugar chain are categorized within the cellobiohydrolase II (CBH2) class. This difference in activity is evident in their structures, with CBH1 members containing a glycoside hydrolase type 7 catalytic domain at the N-terminus followed by a carbohydrate binding domain near the C-terminus. These features are inverted in the CBH2 class, with a carbohydrate binding domain near the N-terminus and a glycoside hydrolase type 6 catalytic domain at the C-terminus. According to the Carbohydrate Active enZYmes (CAZY) database, there are 2,195 potential glycoside hydrolase family 6 (GH6) gene candidates, of which 210 are found in eukaryotic organisms. Certain cellobiohydrolases are incorrectly annotated as being type II cellobiohydrolases (within the GH6 class) but are rather predicted to be type I cellobiohydrolases (within the GH7 class) according to their structures, and this has resulted in many published articles that incorrectly classify specific exo-cellobiohydrolases in the wrong family. We confirmed the correct glycoside hydrolase family assignment for the cellobiohydrolase candidate genes cloned using protein domain prediction software. The CBH2 enzyme from Trichoderma reesei does not hydrolyze 4-methylumbelliferyl-β-D-aglycones of either glucose or cellobiose units, but does cleave 4-methylumbelliferyl-β-D-glycosides with longer glucose chains, such as cellotriose, cellotetraose and cellopentaose. [33,34] We took advantage of this information to challenge L. starkeyi transformants of Trichoderma reesei CBH1 and CBH2 to hydrolyze 4-methylumbelliferyl beta-D cellotetraoside (4-MUC) and 2-chloro-4-nitrophenyl-beta-D-cellotetraoside (CPNPG4), FIGS. 5A and 5B. These assays involve an increase in fluorescence or absorbance following cleavage of the cellotetraoside, respectively. In both cases, the CBH2 transformant supernatant displayed activity, indicating an active secreted CBH2 enzyme, whereas little or no activity was observed in the wild-type and CBH1 derived supernatants. While the fluorescence based 4-MUC method is more sensitive than the CPNPG4 method, 4-MUC is significantly more expensive than CPNPG4. Moreover, the 4-MUC assay involved a final pH adjustment to measure fluorescence, which was not needed when screening using CPNPG4. Therefore, we elected to use the CPNPG4 methodology to screen CBH2 transformants. A total of 93 TrCBH2 transformants in the NRRLY-11557 background were screened using the CPNPG4 methodology.

A second CBH2 enzyme optimized for expression in L. starkeyi originally derived from Aspergillus flavus (A/CBH2) was also transformed and evaluated using the CPNPG4 based assay, but none of the transformant supernatants had any detectable activity (data not shown). The lack of activity in the TrCBH1 and A/CBH2 transformant supernatants was likely the result of inactive enzymes, despite being codon optimized for high expression in L. starkeyi and containing a functional secretion signal and promoter. The marginal activity observed in the CBH1 transformant supernatant against the 4-MUC compound is likely due to the more sensitive nature of this assay. This demonstrates that protein folding, glycosylation, or other events are important considerations that dictate enzyme function in L. starkeyi. It is difficult to predict which enzymes will be inactive when heterologously expressed in this organism, or the source(s) of enzyme impairment. For these reasons, more than one type of each class of enzymes (CBH1, CBH2, endoglucanase, and endoxylanase) were evaluated for activity, with more than one type of assay employed when possible.

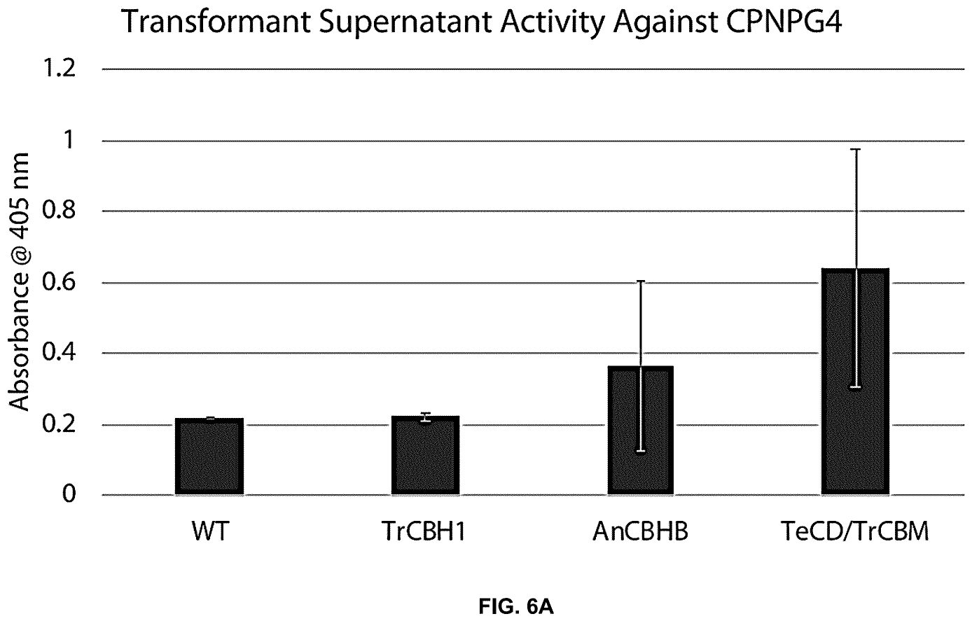

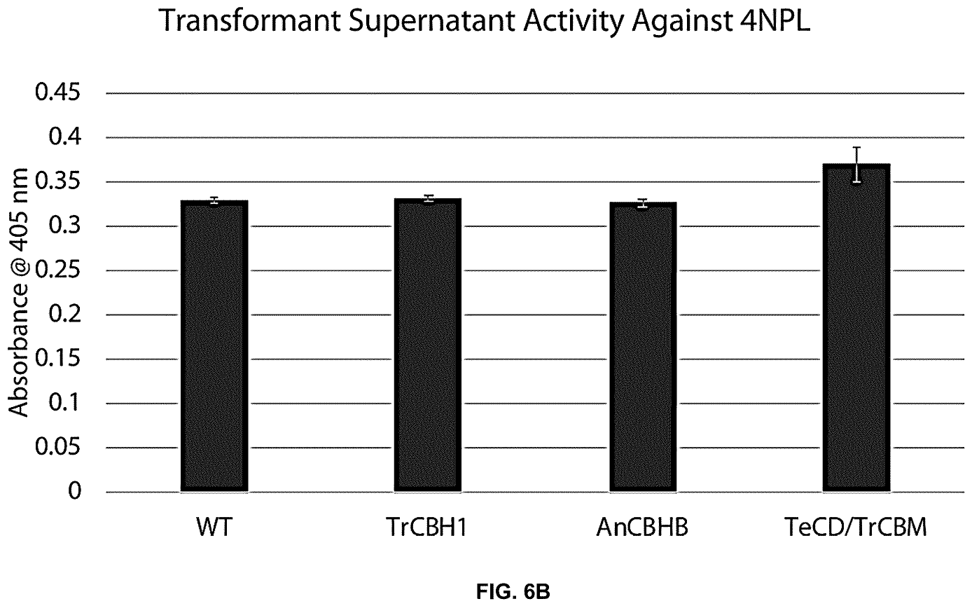

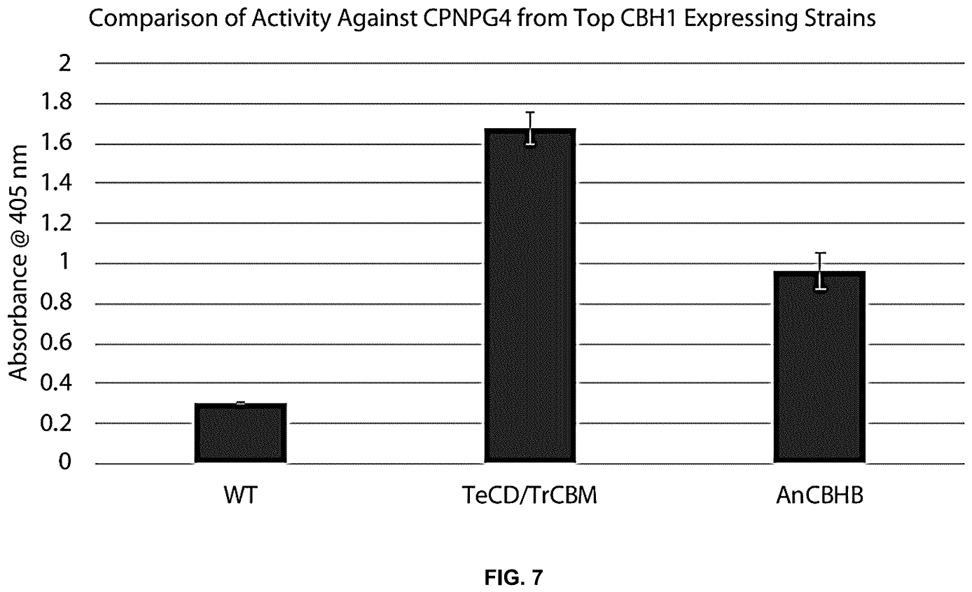

In order to obtain a functionally secreted CBH1 enzyme, two additional candidates were identified, codon optimized, cloned, and transformed into L. starkeyi. These included the CBHB enzyme from Aspergillus niger, and a chimeric enzyme including the catalytic domain from Talaromyces emersonii and the carbohydrate binding motif from Trichoderma reesei (TeCD/T/CBM). [35] A total of 123 transformants were obtained from the AnCBHB cassette, while 76 transformants were obtained from the TeCD/TrCBM chimera. In a preliminary screen, 2-chloro-4-nitrophenyl-beta-D-cellotetraoside (CPNPG4) and 4-nitrophenyl-β-lactoside (4NPL) were subjected to transformant supernatants from both constructs and monitored for substrate cleavage as indicated by an increase in absorbance at 405 nm relative to the wild-type supernatant. Following a 19-hour overnight incubation, activity was clearly detected against the CPNPG4 substrate from both constructs, and a marginal activity was detected against the 4NPL substrate from the TeCD/TrCBM chimera (FIGS. 6A and 6B). Supernatants from TrCBH1 transformants were also included as a control. The large error bars reflect how the unique site of genomic integration influences heterologous enzyme expression in each transformant. Considering the higher sensitivity of the CPNPG4 assay, we proceeded screening AnCBHB transformants with this assay over the 4NPL assay. Both preliminary and competition screening between the top 4 strains of each construct revealed a higher intrinsic activity of the TeCD/TrCBM chimera over AnCBHB using this substrate (FIG. 7).

### Endoglucanases

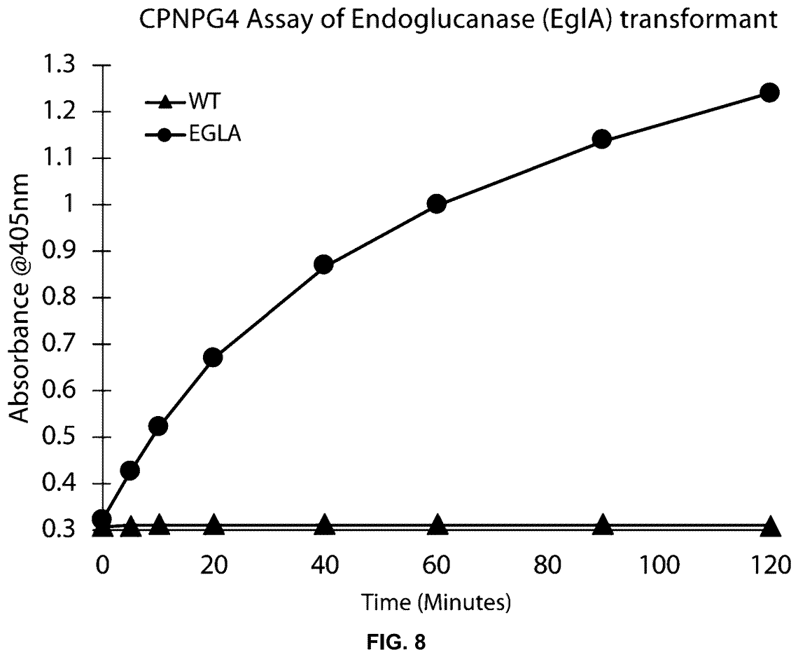

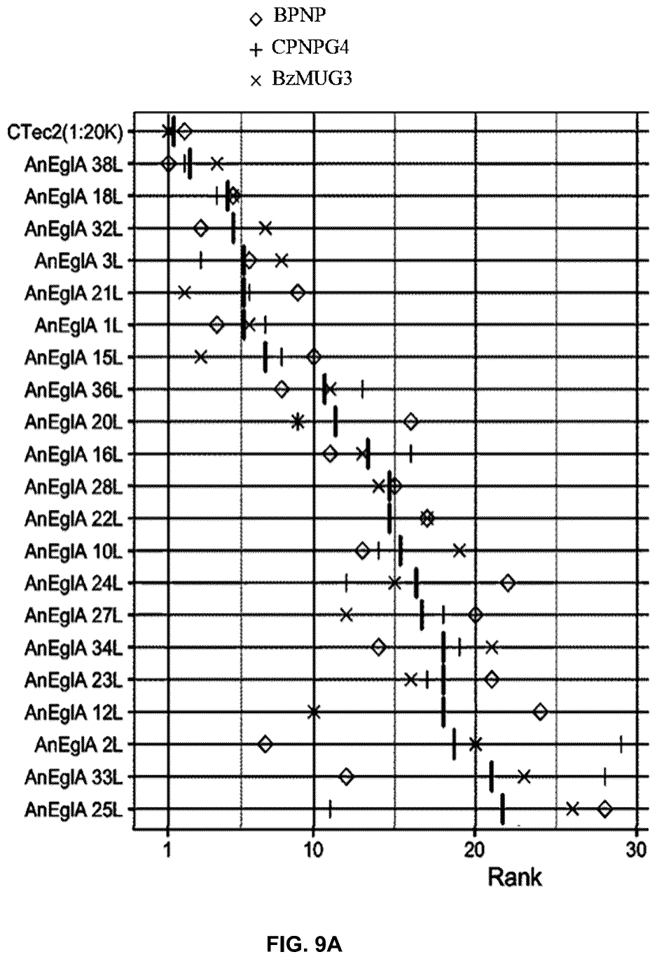

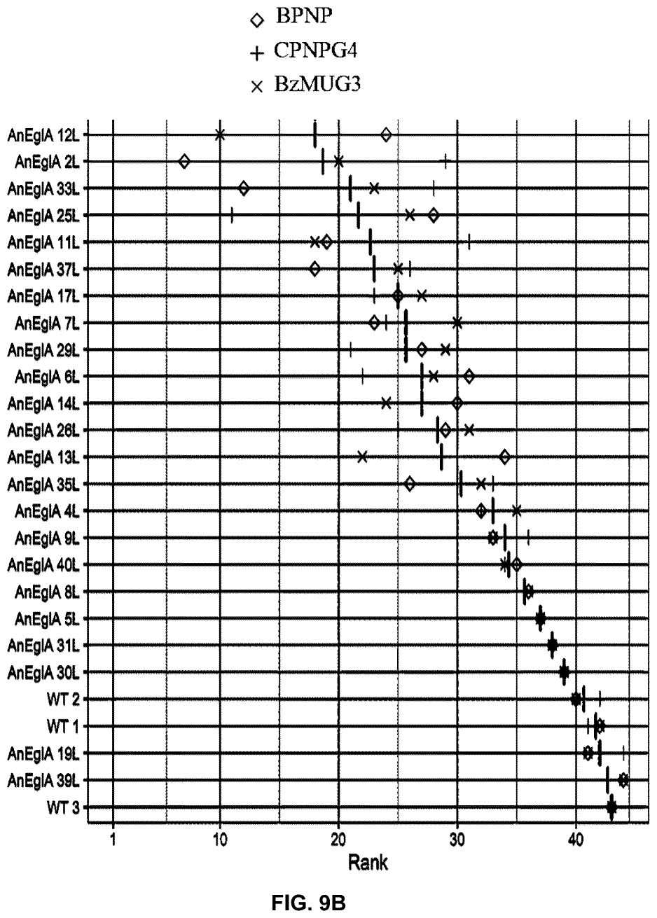

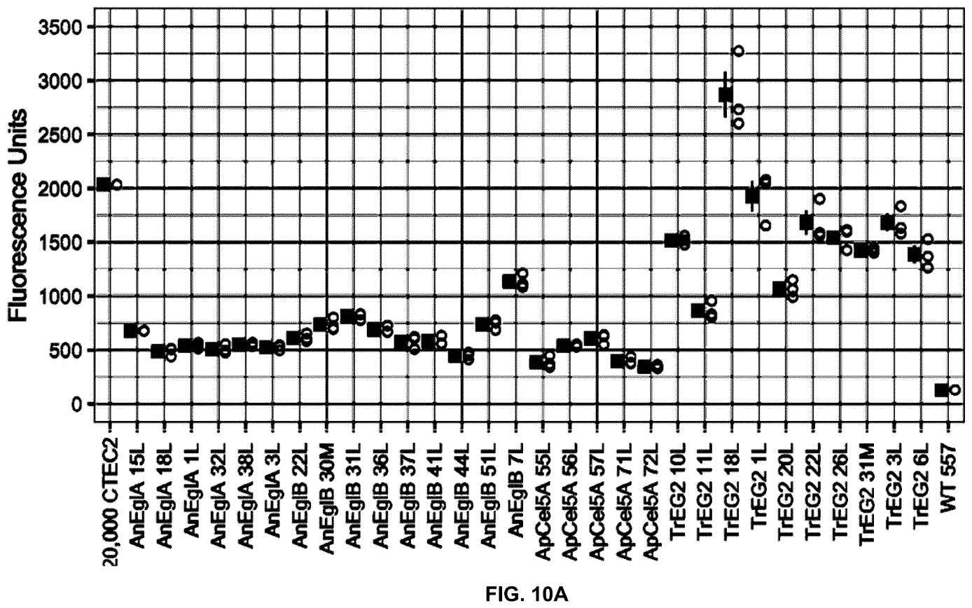

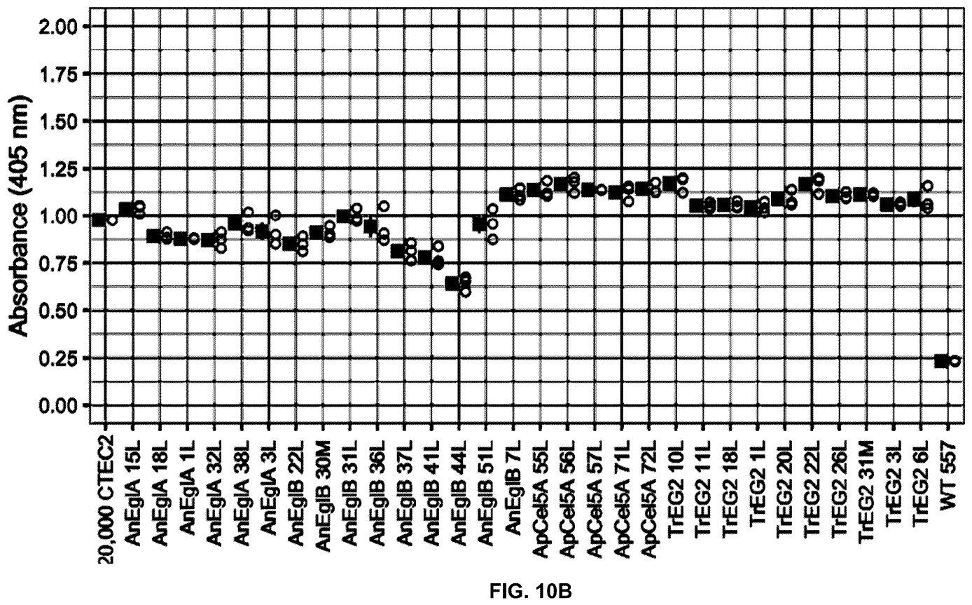

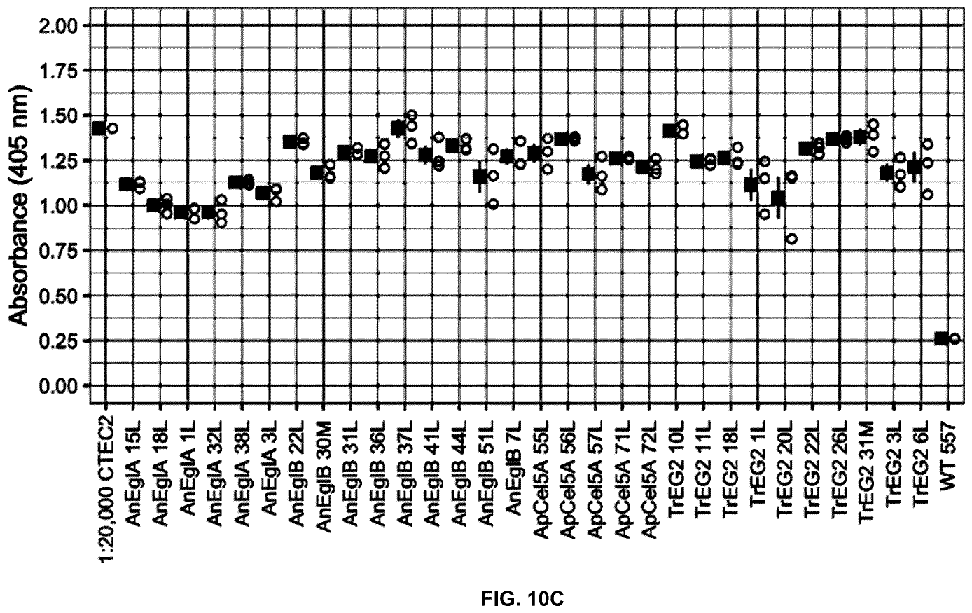

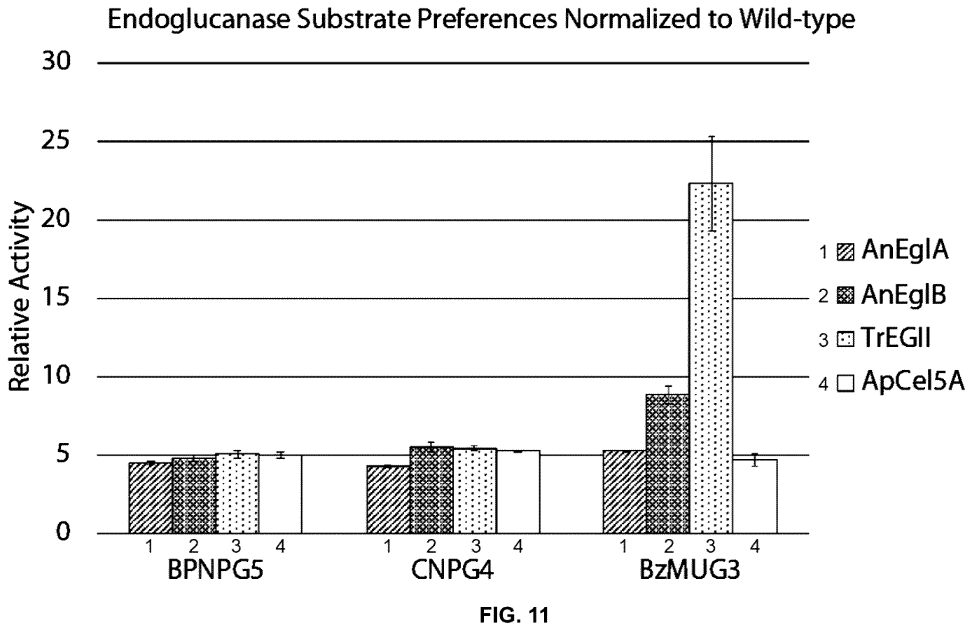

Endoglucanases were the next cellulase class evaluated, which cleave the internal glycoside bonds in cellulose. The specific endoglucanases cloned into L. starkeyi were derived from 3 different species (Trichoderma reesei, Aspergillus niger, and Aureobasidium pullulans) and included enzymes from different glycoside hydrolase families (S or 12) that contain the presence or absence of a carbohydrate binding domain (CBD). Collectively, over 300 endoglucanase transformants were obtained. Commercially available kits exist for detecting and measuring endoglucanase activity. One example is absorbance based and depends on cleavage of 4,6-O-(3-ketobutylidene)-4-nitrophenyl-β-D-cellopentaside (BPNPG5). Another is fluorescence based and depends on cleavage of 4,6-O-Benzylidene-4-methylumbelliferyl-β-cellotrioside (BzMUG3). These were both modified for high-throughput screening purposes. Unexpectedly, when we included a transformant supernatant derived from a strain overexpressing the Aspergillus niger EglA in the CPNPG4 assay intended as a negative control, we observed activity towards this compound as well (FIG. 8). Other endoglucanases were then evaluated for activity against CPNPG4 and were also found to cleave the substrate. In order to compare the activities of different endoglucanase enzymes among themselves and between different assays, we modified all three endoglucanase assays (BPNPG5, BzMUG3, and CPNPG4) for high throughput screening purposes in 96 well plates. This allowed comparing the results of all three assays against the four types of endoglucanases (AnEglA, AnEglB, ApCel5A, and TrEGII) secreted in the superatant of engineered L. starkeyi strains. All three assays were able to sufficiently segregate transformants based on activity against the respective substrates. Importantly, each assay resulted in the selection of the same strains with high activity, with few exceptions (FIGS. 9A and 9B). As expected, the activity of transformants was widely distributed based upon the unique genetic locus each cassette had integrated within, yet the variance within each true replicate was low across the assays (FIGS. 10A-10C). The utilization of three different yet complementary assays against four types of endoglucanases enabled a comparison between the activity of each endoglucanase relative to each other and the preference of each enzymes toward the substrate used in each assay (FIG. 11). For example, the top AnEglA endoglucanase transformants consistently displayed less activity against all three substrates relative to the top transformants of the other enzymes when normalized to the wild-type. On the other hand, the fluorescence based BzMUG3 assay yielded higher overall activities for all enzymes, likely due to its higher sensitivity. In fact, this assay revealed very strong activity in the top TrEGII transformants followed by a moderate level of activity of the top AnEglB transformants. The absorbance-based BPNPG5 and CPNPG4 assays yielded similar results from the top transformants of each endoglucanase relative to the wild-type.

### Xylanases

Fiber derived from stillage and other corn-based processes contains a significant amount of hemicellulose. We therefore created cassettes that overexpress xylanases for integration into the top cellulolytic strains, yet different glycoside hydrolase (GH) families of hemicellulases have different activities. For example, GH10 xylanases have been reported to be highly active on short xylo-oligosaccharides, and soluble and branched xylans. GH11 xylanases, on the other hand, are highly active on unsubstituted regions of xylan and insoluble xylan, with reduced activity on decorated xylans. Moreover, members of each class may contain a carbohydrate binding module (CBM) and it is unknown what effect removing this has on enzyme activity. Considering this, we created five codon optimized xylanase overexpression cassettes to secrete enzymes from different GH families, as described in Table 2. The first is derived from Trichoderma harzanium XYN2 (ThXYN2) and is classified in the GHI1 family of endoxylanases and contains a CBM. The second originates from Aspergillus niger XYNA (AnXYNA) and belongs to the GH10 family and lacks a CBM. The third, TXYND, originating from Talaromyces funiculosus, is also within the GH10 family but contains a CBM. TAXYND has had its kinetics reported in the presence of several substrates, including birchwood xylan (Kₘ=2 mg/mL, Vₘₐₓ=130 μmol/min/mg enzyme), insoluble wheat arabinoxylans (Kₘ=11 mg/mL, Vₘₐₓ=535 μmol/min/mg enzyme), and has an optimal pH between 4.2-5.2. The fourth enzyme, TJXYNB, is also derived from Talaromyces funiculosus and predicted to contain a CBM but is within the GH11 family. The last enzyme is derived from Fusarium graminearum (FgXYNC) and is classified within the GH10 family and does not contain a predicted CBM. FgXYNC has been shown to have a high turnover rate (K_{cat}=684 l/s) and low Kₘ (8.4±0.4 mg/mL) compared to other xylanases endogenous to Fusarium graminearum. All enzymes are predicted to have secretion signals, which were removed and replaced with native L. starkeyi secretion signals in the final constructs. Transformant pools of each cassette were then evaluated for xylanase activity by modifying a commercially available kit for measuring xylanase activity. Specifically, in the presence of an active xylanase, the compound 4,6-O-(3-ketobutylidene)-4-nitrophenyl-β-D-45-glucosyl-xylopentaoside (Xy1X6) is cleaved, resulting in an increase in absorbance within 400-405 nm.

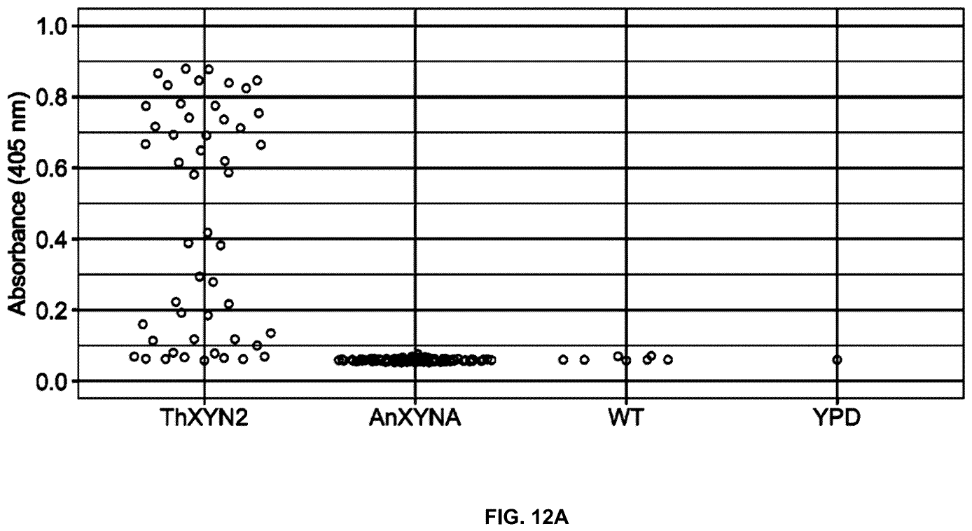

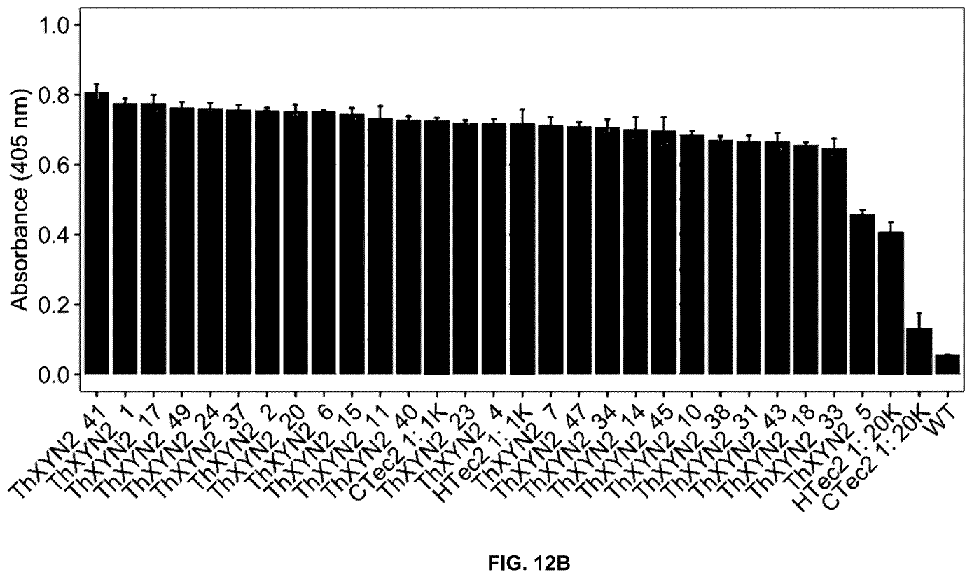

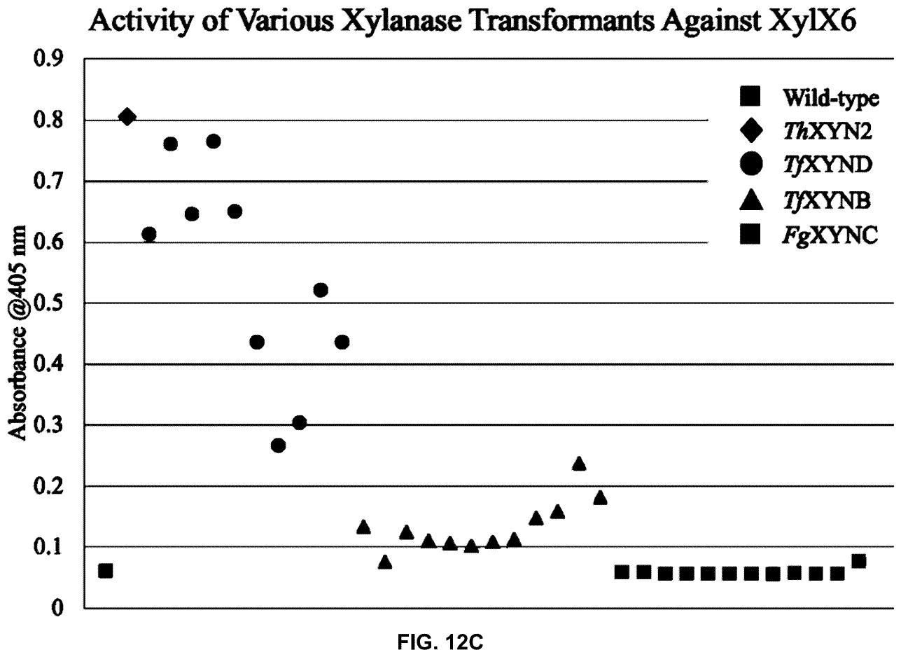

We compared the activities of ThXYN2 and AnXYNA from a pool of 96 and 50 transformants of each, respectively. The results of this screen are shown in FIG. 12A. The endoxylanase activities of ThXYN2 transformants ranged widely, which is consistent with the unique genetic loci integration site in each transformant influencing expression levels of the enzyme. Even though the activities of each transformant varied widely, the variance across replicates of each transformant's activity was low (FIG. 12B). On the other hand, we were unable to observe endoxylanase activity using this assay for any of the AnXYNA transformants. During this time, construction of the other xylanase cassettes was completed and transformants of each were obtained and compared using the same assay (FIG. 12C). From the 3 additional xylanases screened, the TAXYND enzyme had the highest activity, being somewhat comparable to the ThXYN2 counterpart. The 7/XYNB enzyme had marginal yet detectable activity, whereas the ÆgXYNC enzyme was non-functional. Taken together, examples of both GH10 (TAXYND) and GH11 (ThXYN2) classes of xylanases were functionally expressed in L. starkeyi. Two enzymes (FgXYNC and AnXYNA) belonging to GH10 were non-functional. The presence or absence of a CBD on the enzymes evaluated did not seem to correlate with the amount of activity detected. Based on these results, it is difficult if not impossible to determine which xylanases will be functionally expressed in L. starkeyi unless done so empirically, as demonstrated here.

### Additional Proteins

Another class of carbohydrate active proteins that can be engineered into the strains of the invention are accessory enzymes and proteins, which fall into several subclasses. These include the lytic polysaccharide monooxygenases (LPMOs), swollenins, and expansins. The expression and subsequent secretion of these accessory proteins in a cellulolytic and/or hemicellulolytic strain should enhance fiber degradation. Depending on the specific subclass of enzymes, LPMOs mediate cleavage of the C1 and/or C4 carbon of the sugar substrate. The products formed (ketoaldoses and aldonic acids) facilitate assay development for measuring LPMO activity, but it is also possible to monitor H₂O₂ formation in the presence of a reductant, such as ascorbate.

Another class of enzymes that can be engineered into the strains of the invention are arabinofuranosidases. Enzymes of particular interest are α-L-arabinofuranosidase AxhA and α-L-arabinofuranosidase B AbfB. AxhA hydrolyzes aryl α-L-arabinofuranosides and cleaves arabinosyl side chains from arabinoxylan and arabinan. AbfB catalyzes the hydrolysis of α-1,2-, α-1,3- and α-1,5-L-arabinofuranosidic bonds in L-arabinose-containing hemicelluloses such as arabinoxylan and L-arabinan. The expression and subsequent secretion of the arabinofuranosidases in a cellulolytic and/or hemicellulolytic strain should enhance fiber degradation.

Other enzymes that can be engineered into the strains of the invention are GH30 xylanases and acetylxylan esterases. Xylanase B from Talaromyces cellulolyticus (ToXyn30B) is a bifunctional enzyme with glucuronoxylanase and xylobiohydrolase activities. ToXyn30B strictly recognizes both the C-6 carboxyl group and the 4-O-methyl group of the 4-O-methyl-α-D-glucuronyl side chain by the conserved residues in GH30-7 endoxylanases. Acetylxylan esterase (or acetyl xylan esterase) catalyzes the deacetylation of xylans and xylo-oligosaccharides. Acetylxylan esterase A (AXEI AAPNC; protein name ANI_1_1204124) belongs to the alpha/beta hydrolase (AB-hydrolase) superfamily of conserved protein domains. Carbohydrate esterases are not yet assigned to a CAZY (Carbohydrate Active enZYmes) family. The expression and subsequent secretion of the GH30 xylanase and acetylxylan esterase in a cellulolytic and/or hemicellulolytic strain should also enhance fiber degradation.

### Protein Stability in First Generation Strains

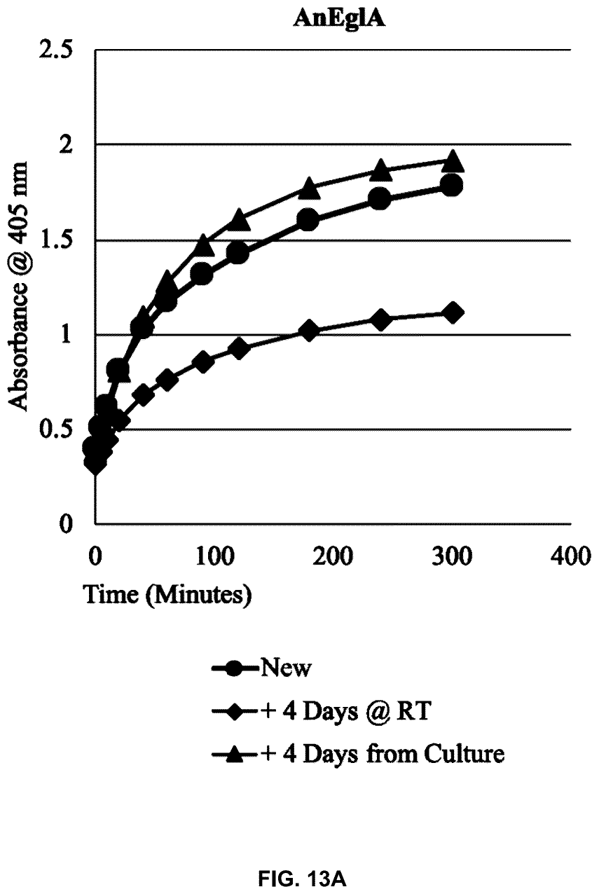

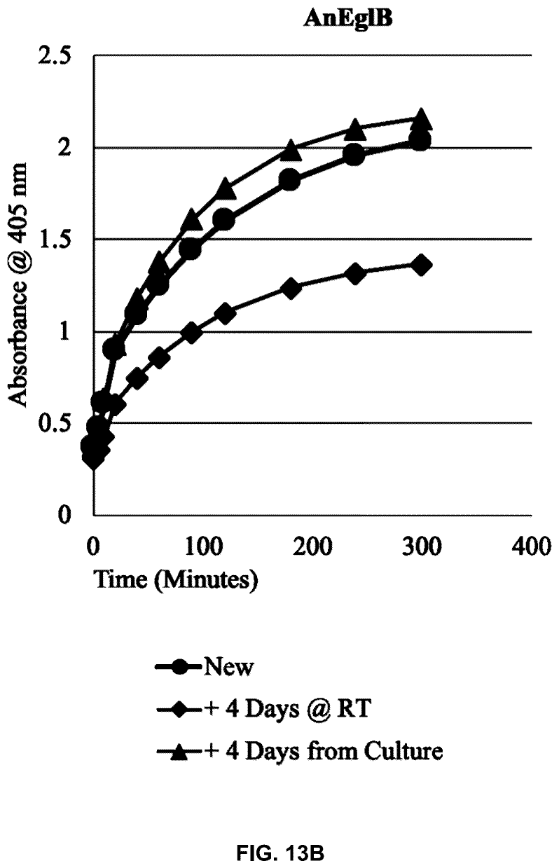

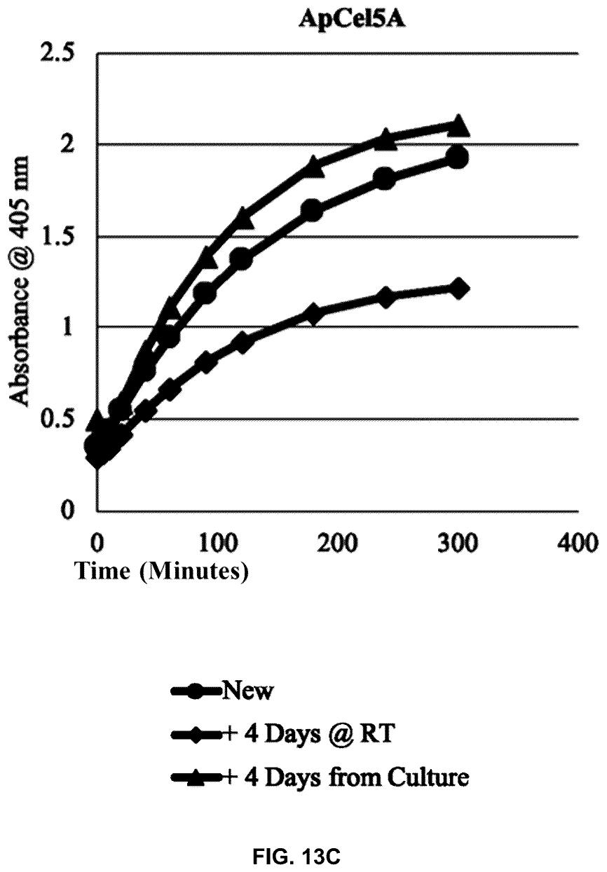

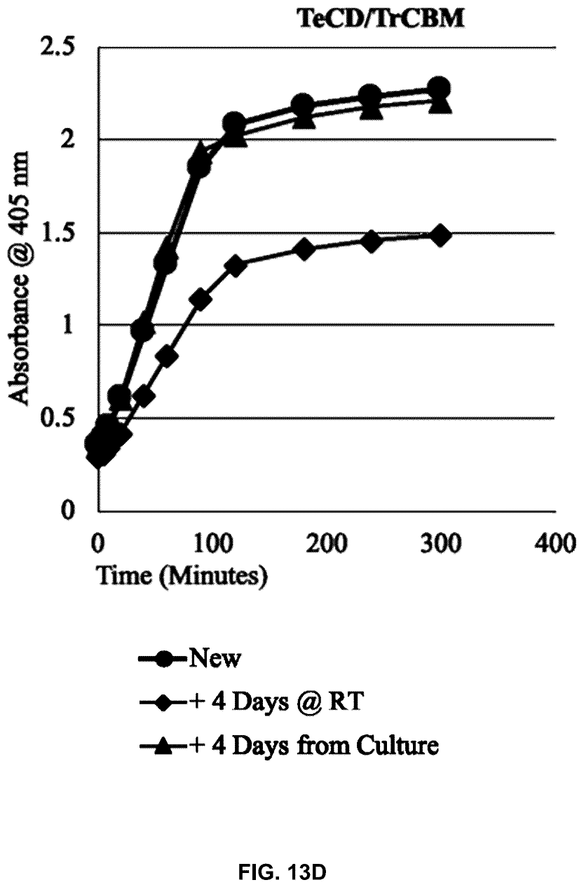

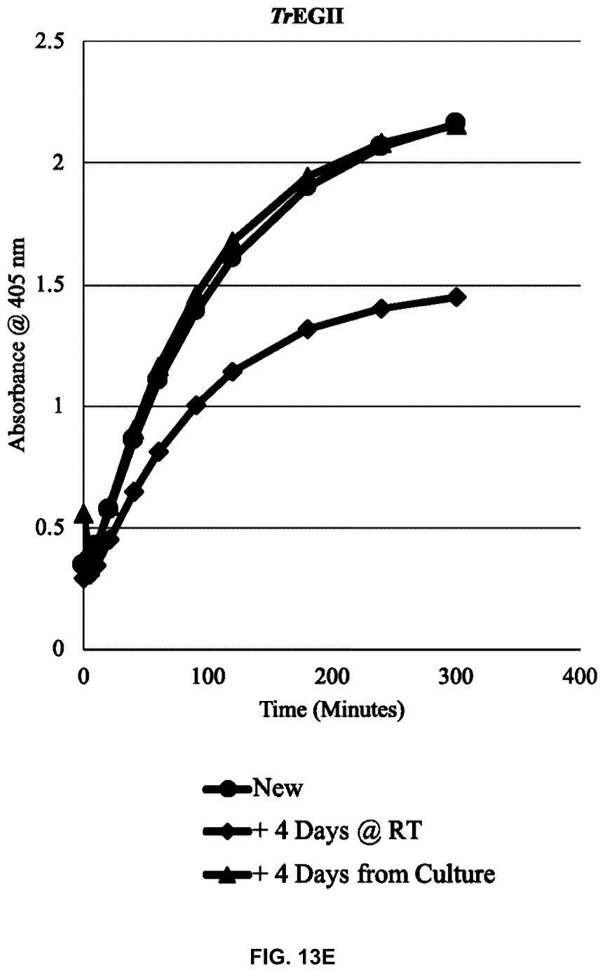

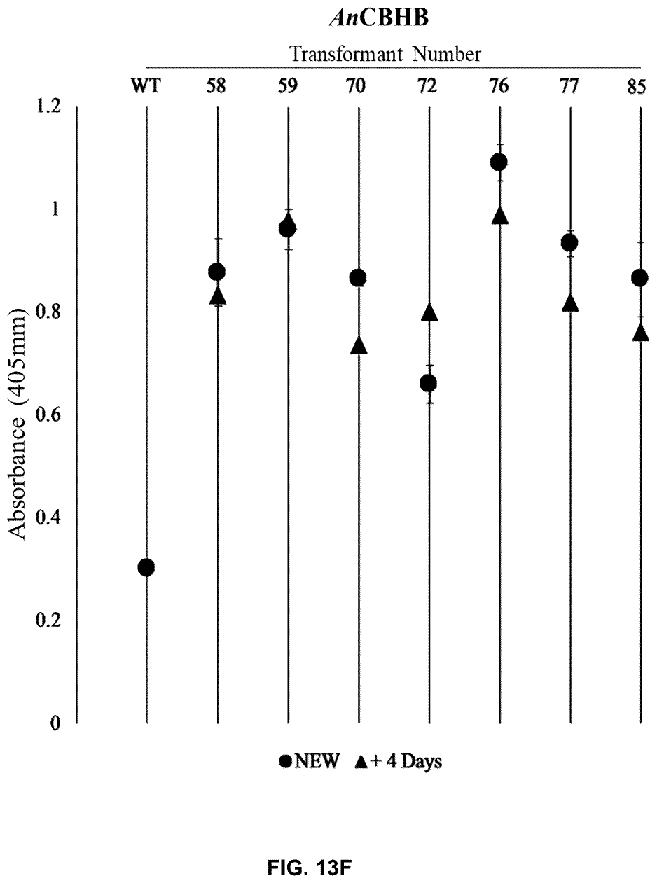

In order to evaluate the stability of the secreted cellulases from first generation (single enzyme transformants) of L. starkeyi, we performed a stability test from single top transformants of cellbiohydrolase I, cellobiohydrolase II, and endoglucanase cassettes using 2-chloro-4-nitrophenyl-beta-D-cellotetraoside (CPNPG4) as a substrate (FIGS. 13A-13F). Cultures were grown to saturation in YPD for 3 days, after which the CPNPG4 assay was performed on a sample of culture supernatants. This supernatant was stored at room temperature for an additional 4 days. Likewise, culture supernatants were prepared from the original cultures that had been incubating at 30° C. during the same 4-day period. Both the original culture supernatants and those freshly prepared from the 7-day old cultures were then evaluated in parallel.

In all cases, activity against CPNPG4 was detected, but to different degrees. All of the original culture supernatants that underwent a 4-day storage period at room temperature showed decreased activities. However, cellulase activities from supematants freshly prepared from the same 3-day or 7-day old cultures were comparable, and some of the samples derived from 7-day old cultures demonstrated a higher activity than samples from the 3-day old culture. This is an important result, since the preferred bioprocess involves growing cells on com fiber derived material in the presence of secreted cellulases, and not performing the enzymatic hydrolysis using previously isolated enzyme followed by culture outgrowth. The apparent increase in activity of these samples could be due to a more concentrated enzyme sample as a result of evaporative loss and/or continued secretion of previously synthesized enzymes. Release of enzymes as a result of cell lysis is unlikely, as we observed no evidence of cell lysis by microscopy.

Enhancing the Secretory Pathway in L. starkeyi

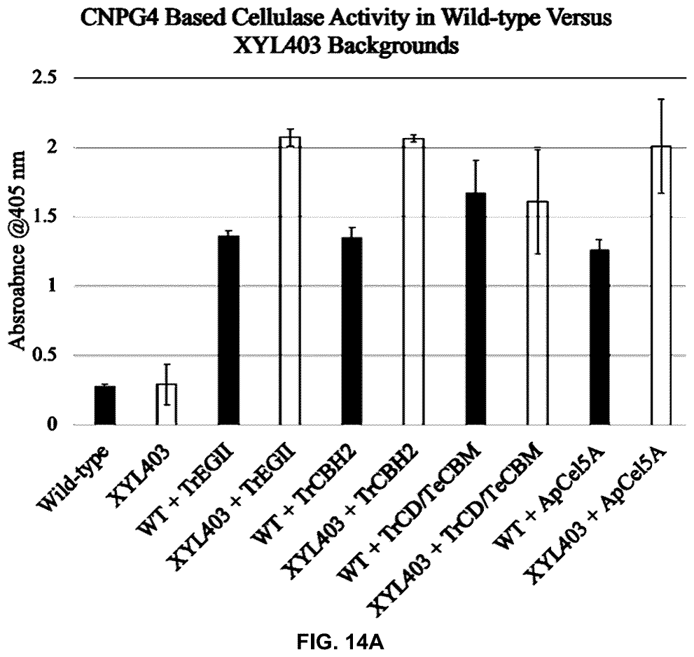

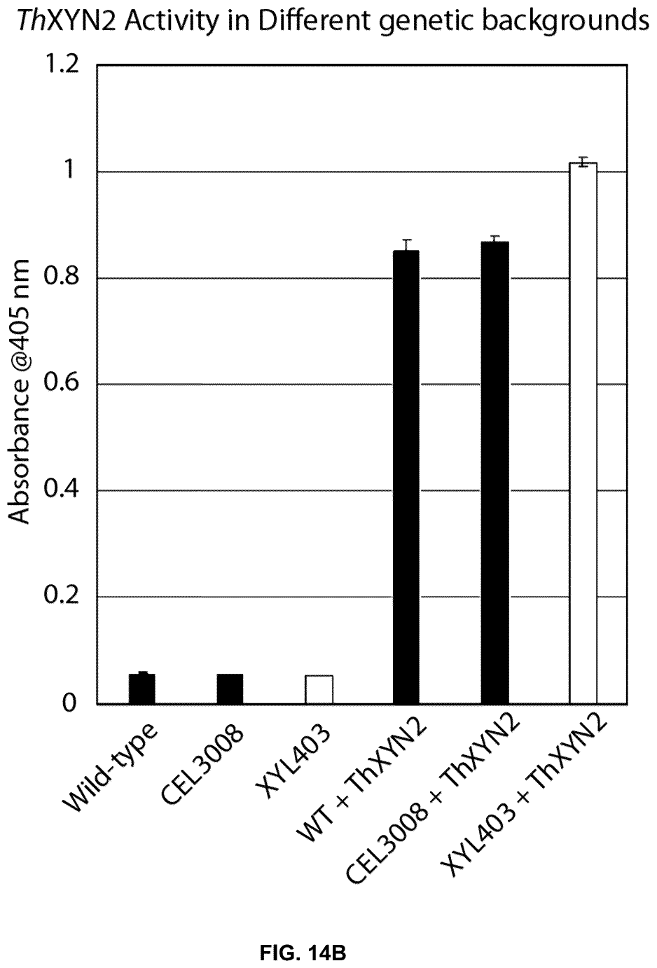

We have found an enhancement in the activity of certain cellulases and xylanases in strains engineered with lipogenic enzymes compared to strains lacking such enzymes. This difference is likely due to an enhancement in the secretion of these proteins, and not by a difference in their expression or stability. This effect is most pronounced when comparing single transformants overexpressing THEGII, TrCBH2, and ThXYN2 in the wild-type versus the highly lipogenic XYL403 background (FIGS. 14A and 14B). We postulate that the increased synthesis of lipid results in expansion of membranes comprising the endoplasmic reticulum and Golgi apparatus, which in turn facilitates protein secretion.

### Strain Development Strategy Utilizing Yeast Mating

Traits in two different Lipomyces starkeyi transformants can be consolidated into a single strain by mating. Since Lipomyces starkeyi is homothallic, mating can occur within the parental strains as well as between them. Therefore, selection of desired progeny is facilitated if each trait is linked to a different resistance marker and spores are germinated on media containing the respective antibiotics to the selection markers. This ensures selection of strains with double drug resistance, (implying that they harbor both traits) and eliminates selection of non-sporulated parental strains and progeny from single strain crosses. Selected progeny can then either be cured, which enables transformation with additional cassettes, or used in another round of crosses. If the second cross is with a strain containing a third resistance marker, then progeny containing all three traits can be selected on media containing all three antibiotics. We refer to this type of mating as a 2×1 cross.

In order to demonstrate the ability to use a 2×1 cross in strain development, we first mated two strains expressing two types of exo-cellobiohydrolases, TrCBH1 and TrCBH₂, to create a single strain overexpressing both (TrCBH1/TrCBH₂). We then crossed this double cellulase expressing strain with individual endoglucanase (AnEglA) transformants. The resulting progeny were capable of growing on plates containing drugs corresponding to all three resistance markers associated with each cellulase cassette, indicating this strategy functions as a practical means to consolidate up to three cassettes into single strains. This is done in two rounds of mating, as attempting to mate three different strains at once, each with a different resistance marker, will only yield progeny with resistance to combinations of two of the drugs.

### Strain Curing and Verification

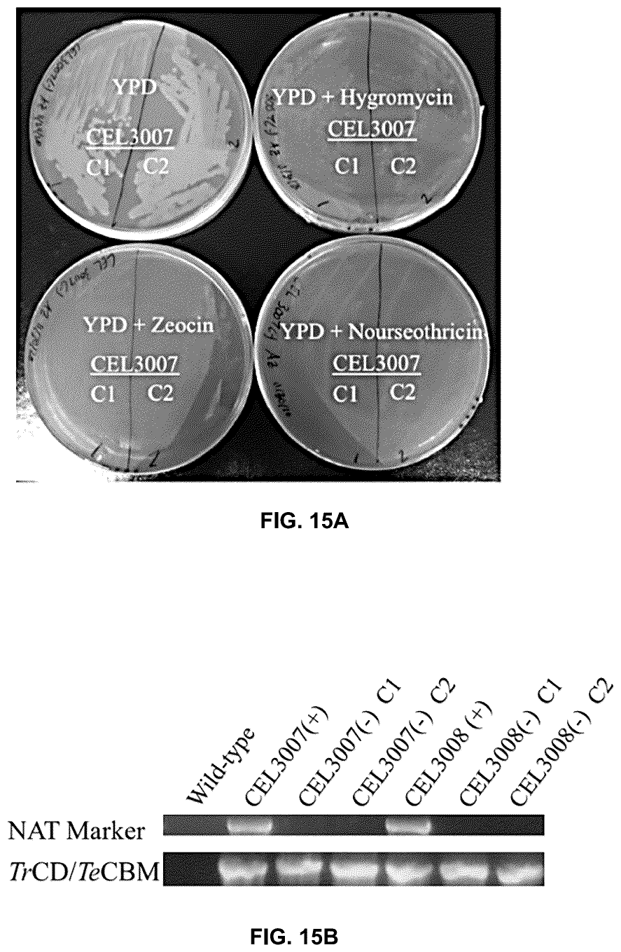

Each cellulase class can use a different resistance marker for selecting transformants. Four markers can be used, with one being reserved for curing (marker removal). A number of cassettes that can be inserted into a strain before it is cured for subsequent transformations is three. Since certain strains, such as CEL3007, CEL3008, and CEL3022 (Table 4) already have three cassettes inserted, they are cured prior to inserting the xylanase construct. We selected these strains for resistance marker removal by transformation with the appropriate curing vector. Following the transformation, microcolonies are selected after 2-4 days of outgrowth on the appropriate selection medium and grown as patches on non-selective media. Marker removal is verified both phenotypically by ensuring cured strains are unable to grow on plates containing the resistance drug of interest (FIG. 15A) and by PCR genotyping (FIG. 15B). In some instances, retention of the cellulolytic cassettes was also confirmed by PCR genotyping (FIG. 15B).

### Fiber Deconstruction by Engineered Strains

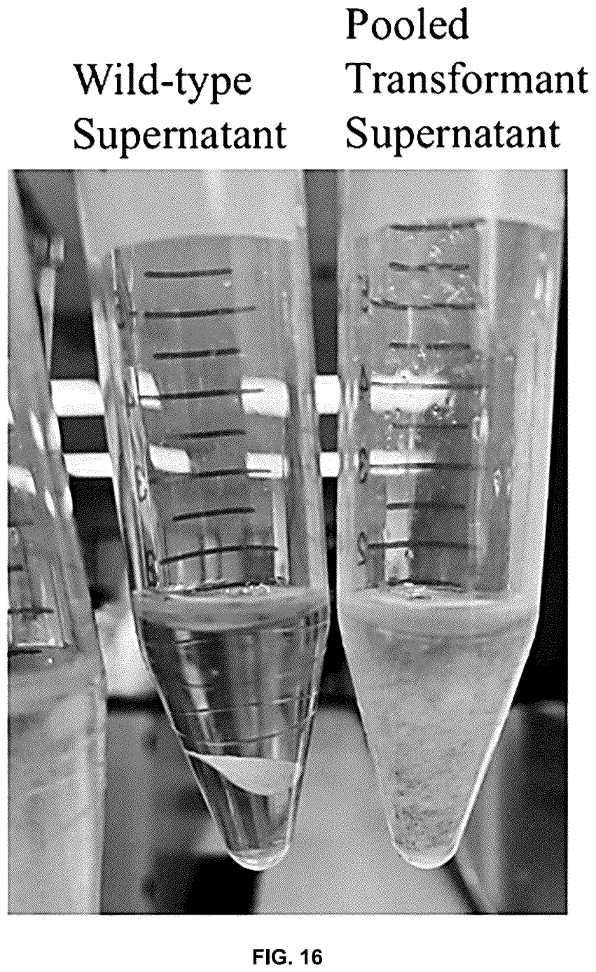

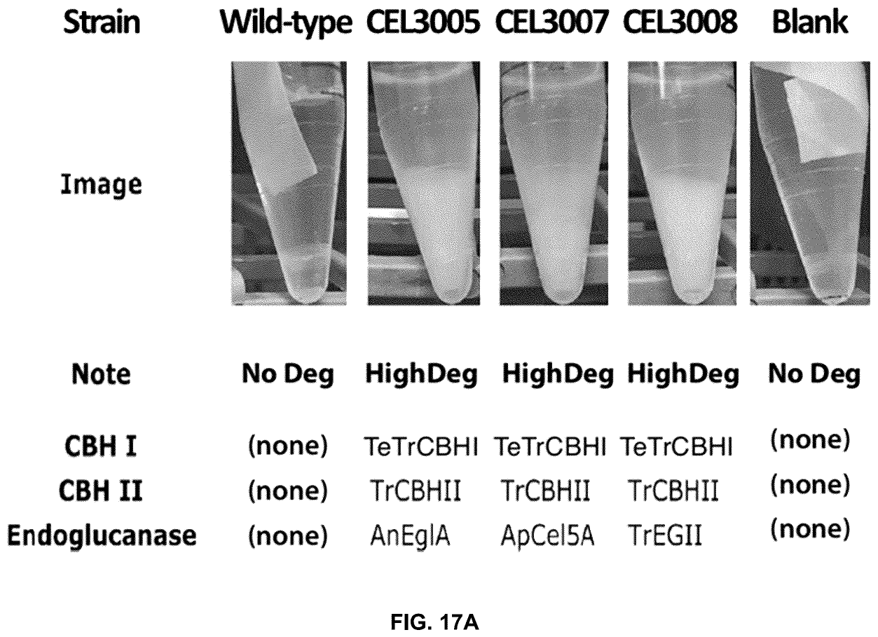

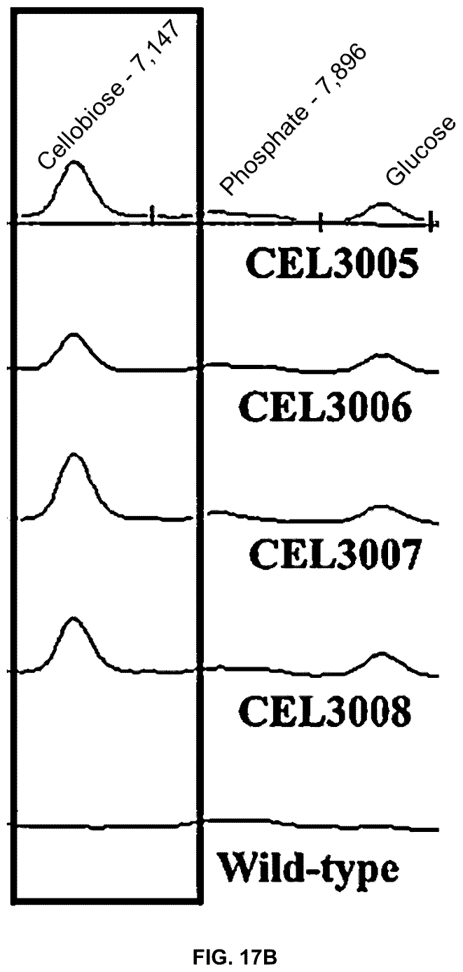

Prior to creating single strains with consolidated cellulase activities, the supernatants of single transformants secreting different cellulase classes were combined in order to demonstrate synergism of their activities against a filter paper substrate. The supernatant pool was incubated at 30° C. for several days with a filter paper chad. A separate sample was also prepared using a wild-type culture supernatant. In the pooled cellulase sample, a significant portion of the filter paper was degraded, whereas essentially all of it has remained intact in the wild-type sample (FIG. 16). This demonstrates that the combined activities of single transformant superatants is sufficient to deconstruct a cellulose-based filter paper substrate, and implied that single strains secreting these enzymes would have a similar effect on this substrate. We used this qualitative filter paper assay to evaluate the cellulolytic activities of mated strains containing more than one type of cellulase integrated within the genome. This assay revealed three strains, CEL3005, CEL3007, and CEL3008 (Table 4), clearly exhibit the ability to degrade cellulose in the form of filter paper (FIG. 17A). HPLC analysis of the supernatants in these samples and others clearly indicated a peak consistent with the presence of cellobiose, which was absent in the wild-type strain (FIG. 17B).

This first set of strains screened in this manner (CEL3001-CEL3008) contained consolidated activities of 3 cellulases created from the 2×1 mating strategy described in the preceding section from the top single transformants of each cassette. Approximately 58 second generation strains were created and screened by the qualitative filter paper method. The top combinations of these were then subjected to the 2×1 crossing approach to create an additional 15 strains (CEL3009-CEL3023). Surprisingly, all of these strains, with the exception of CEL3022, were inferior to CEL3008 in this and other screens. Due to these results, the strains CEL3007, CEL3008 and CEL3022 were “cured” of resistance markers and subsequently used as platform strains for further engineering by inserting additional genes related to fiber deconstruction, as listed in Table 2. Two of these strains, CEL4001 and CEL4002 (Table 4), were derived from the cured CEL3008 strain and expressed and secreted the highly functional xylanase enzyme, ThXYN2.

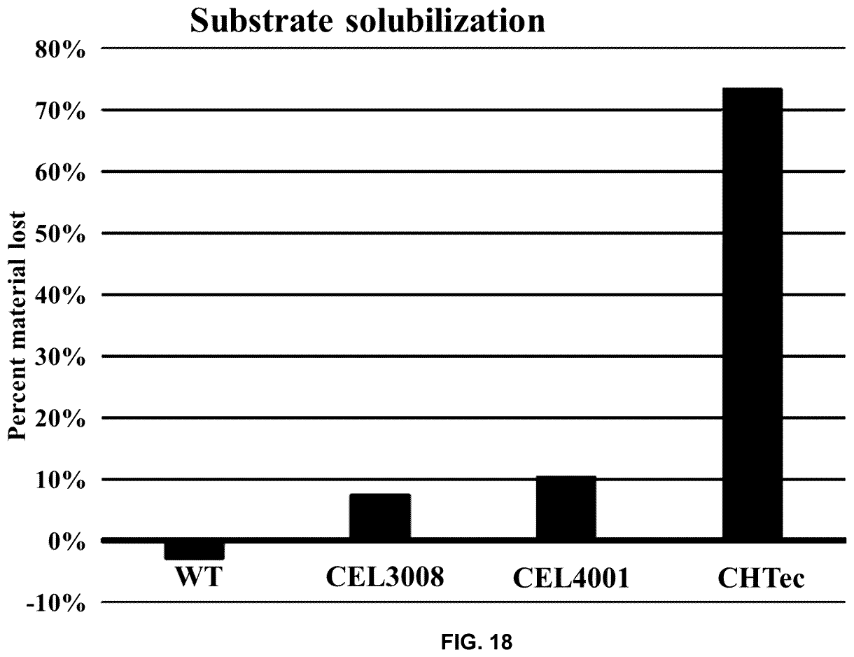

The CEL3008, CEL4001, and CEL4002 strains (Table 4) were then evaluated for their ability to degrade other fiber containing substrates including deacetylated mechanically refined (DMR) com stover from NREL, and isolated corn stillage fiber from a local ethanol plant. In the first example, a neutral detergent fiber (NDF) assay [36] was employed that enables the quantification of cellulose, hemicellulose, and lignin in a plant substrate by using a neutral pH detergent to solubilize proteinaceous and bound cellular material prior to passing the sample through a pre-weighed filter. This assay revealed a marked increase in DMR com stover solubilization by the CEL3008 strain over the wild-type strain, which is further improved by the CEL4001 strain to over 10% solubilization (FIG. 18). When compared with the solubilization exhibited by an equal mixture of commercial enzyme cocktails Cellic™ Ctec2 and Htec2 (for a total of 2% v/v of enzyme cocktail), the supernatants of the CEL3008 and CEL4001 strains solubilize much less of the DMR com stover in the same period. However, these data are not normalized to enzyme abundance, which is presumably much higher in the commercial cocktails. Moreover, the CEL3008 and CEL4001 strains are only secreting 3 and 4 cellulases and/or hemicellulases, respectively. The commercial cocktails presumably contain many more types of glycoside hydrolase families and accessory enzymes.

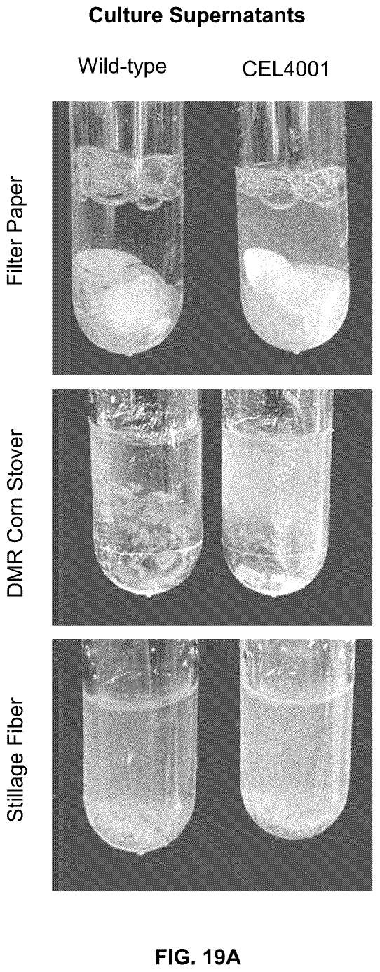

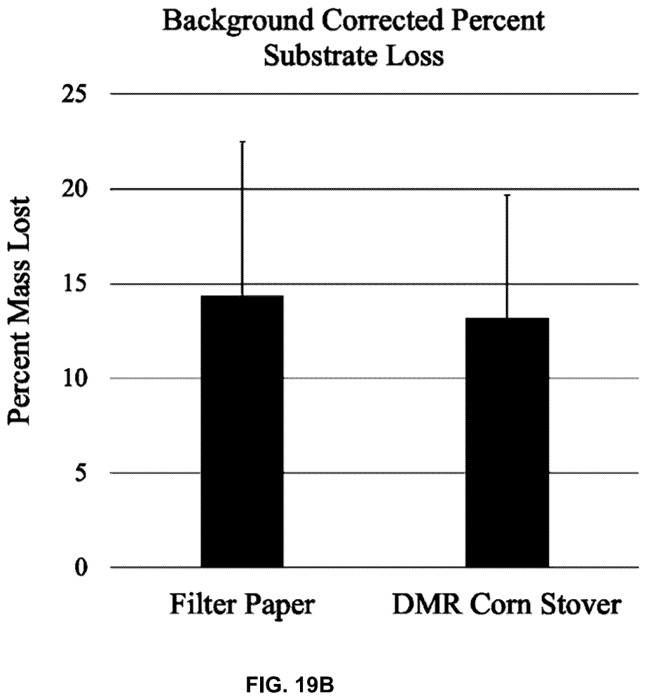

Direct gravimetric measurements of fiber solubilization is a simpler approach than the neutral detergent fiber method described above. Using this technique, deconstruction of the varying substrates is evident after just 24 hours, particularly in the sample containing the DMR com stover (FIG. 19A). Gravimetric analysis demonstrated ≈15% total solubilization of the filter paper and DMR com stover substrates after 5 days at 30° C., although the standard deviation was around 50% in these samples (FIG. 19B). There was also some degradation (about 5%) in the stillage fiber (data not shown). In these studies, a washing step was omitted to prevent additional losses due to the washes themselves. Improved techniques have allowed including a washing step and constant agitation in the procedure, which has reduced the standard deviation across samples.

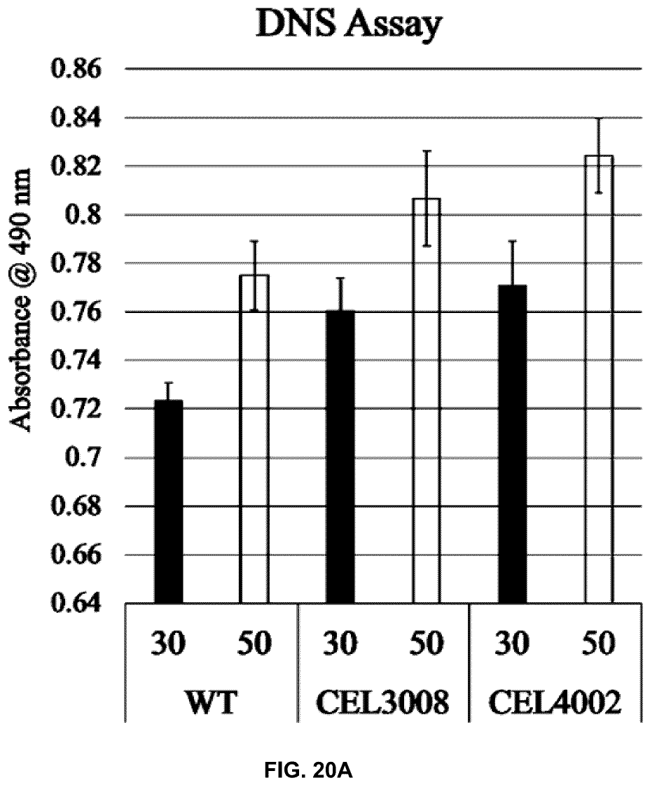

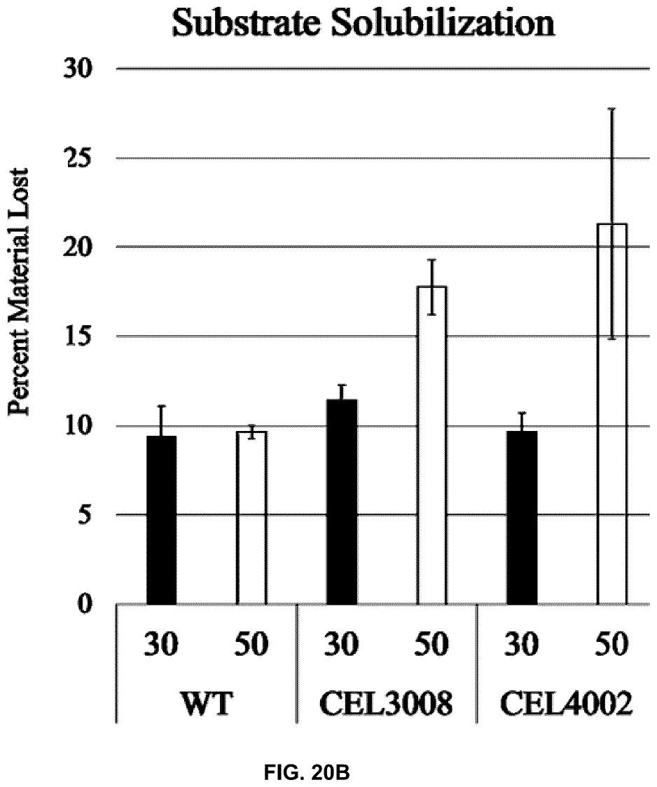

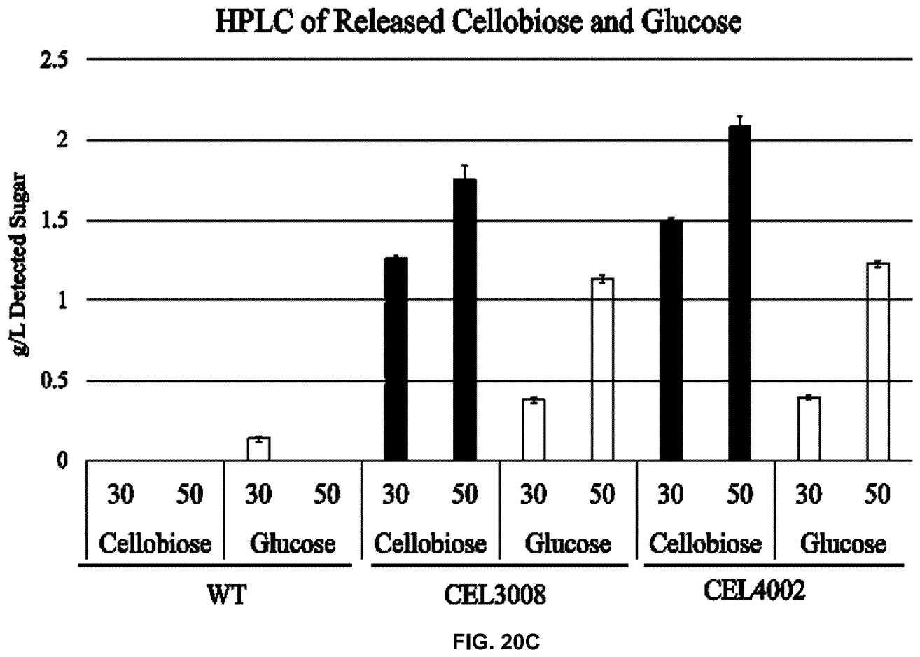

We also evaluated the secreted enzymatic activity of the wild-type. CEL3008, and CEL4002 strains on filter paper by monitoring the reactivity with 3,5-dinitrosalicylic acid (DNS, which reacts with the reducing ends of sugars), percent mass lost (substrate solubilization), and release of soluble sugars by high performance liquid chromatography (HPLC), at 30° C. and 50° C. (FIGS. 20A-20C). In each case, the 50° C. treatment resulted in more sugars released compared to the 30° C. treatment, demonstrating higher kinetics at the elevated temperature without significant denaturation of the enzyme. Moreover, the CEL4002 strain had comparable if not higher activity than the CEL3008 strain. Since the substrate used in these experiments was filter paper, there should be no appreciable xylan present, yet the higher activity of the CEL4002 strain suggests promiscuous activity that promotes cellulose degradation. Lastly, the HPLC results suggest the presence of a native secreted β-glucosidase, since cellobiose is converted to glucose in these samples to the same degree at each temperature, yet no glucose is present in the wild-type derived sample (FIG. 20C).

### Fiber Utilization by Engineered Strains

The saccharification experiments described above were done in the absence of cells. The lack of a sugar “sink” renders the enzymes susceptible to end-product inhibition by the released sugars, preventing further saccharification above a certain concentration. Moreover, it is difficult to extrapolate how much lipid would be produced from the saccharified material, since the process involves simultaneous saccharification and utilization without permitting significant accumulation of sugars in the culture medium. This is best resolved empirically.

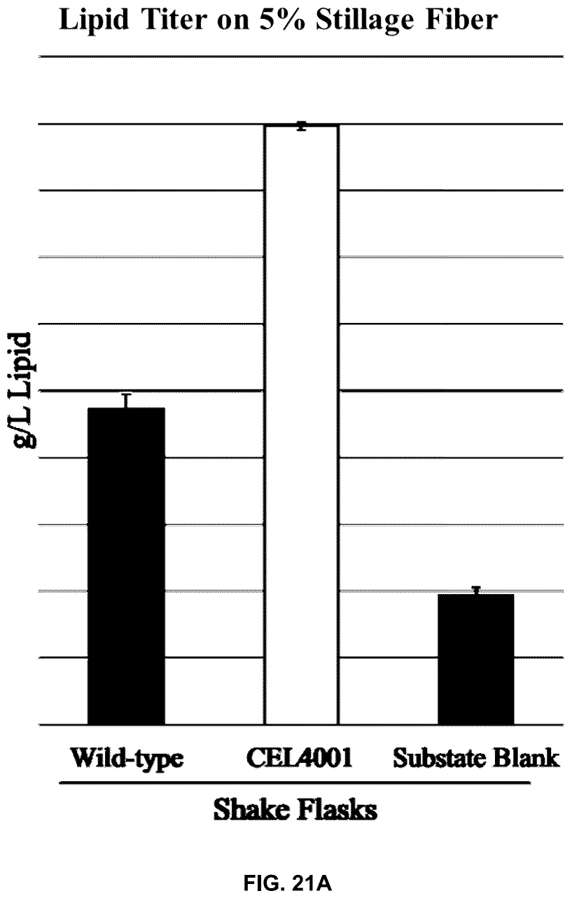

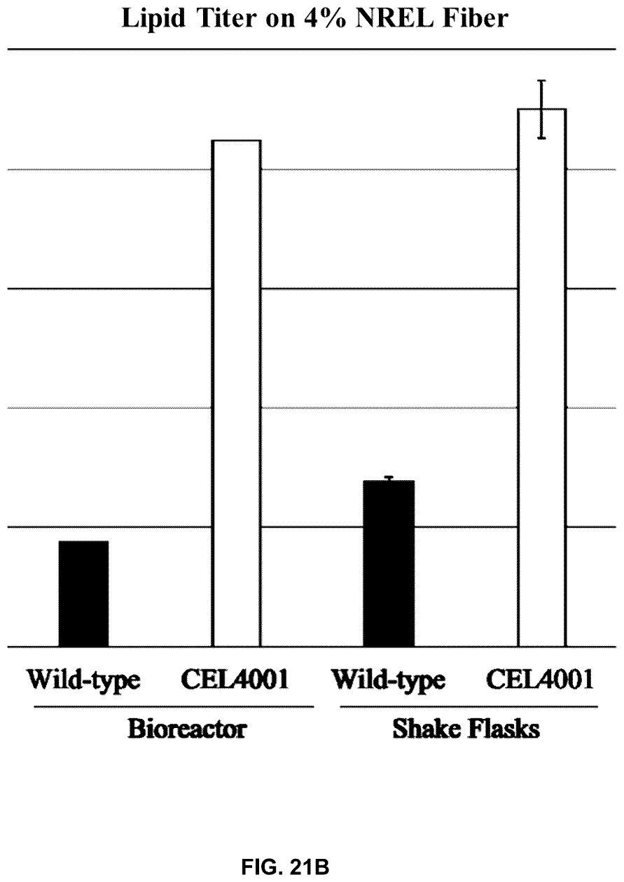

Experiments demonstrated a small yet clear increase in lipid produced in the CEL3008 strain versus the wild-type when grown on a 5% stillage derived fiber material in shake flasks (data not shown). The experiment was repeated using the CEL4001 strain against the wild-type strain. Cultures of CEL4001 showed twice as much lipid than the wild-type strain (FIG. 21A). Since a significant quantity of oil is contributed by the substrate, the actual oil produced by the engineered versus the parental strain is closer to 3× higher.

This result prompted a scale-up experiment in a bioreactor comparing the CEL4001 strain against the wild-type on 4% DMR corn stover. In this experiment, cells were rapidly grown on nitrogen rich media to synthesize and secrete cellulases and xylanases. The enzyme rich culture broth was then isolated from the cells and incubated with DMR com stover to release sugars. After 5 days of saccharification at 50° C., the solids portion of the slurry was removed and the resulting suspension inoculated with wild-type or CEL4001 cells and cultured for two days (or until all of the sugar was depleted). A parallel experiment was also performed in shake flasks using the clarified slurry as a culture broth. In both cases, the engineered CEL4001 strain accumulated 4.8 and 3.3 times more lipid than the wild-type strain (FIG. 21B), respectively. Bioprocess improvements, such as enzyme recycling, and further strain engineering, such as by using the lipogenic hypersecreting XYL403 strain as a platform organism, could increase yields further.

### Arabinofuranosidase Addition

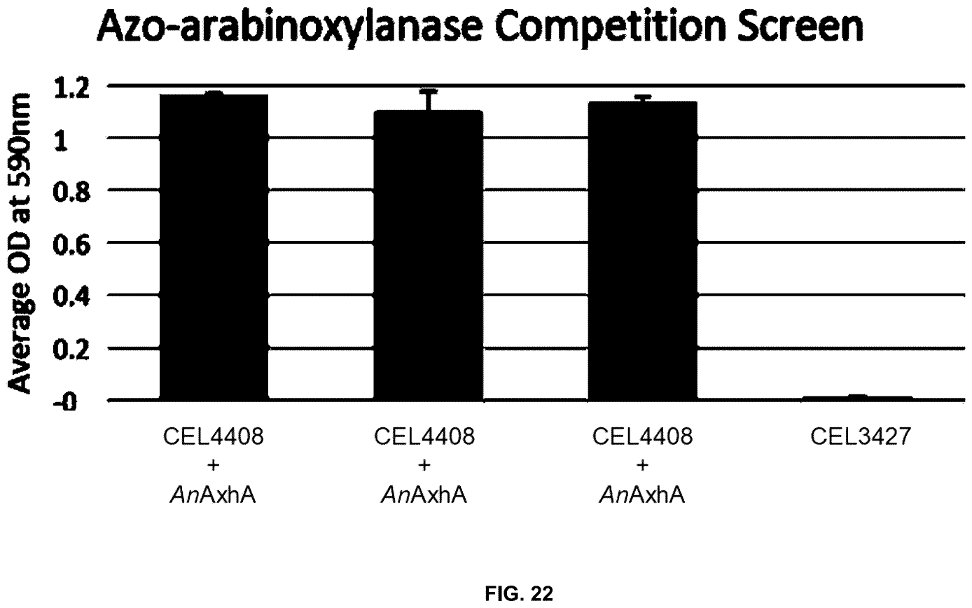

Arabinofuranosidase activity of the CEL3427 strain and three CEL4408 strains with AnAxhA (α-L-arabinofuranosidase) integrations was determined. To measure arabinofuranosidase activity, an azo-wheat arabinoxylanase assay was used. This is a relatively quick colorimetric assay, and strains exhibiting arabinofuranosidase enzyme activity will degrade the substrate and release a blue dye, increasing the OD @590 nm. We performed triplicate assays on our top three candidates where the AxhA gene is integrated into the L. starkeyi genome. Results are shown in FIG. 22.

### Lytic Polysaccharide Monooxygenase Addition

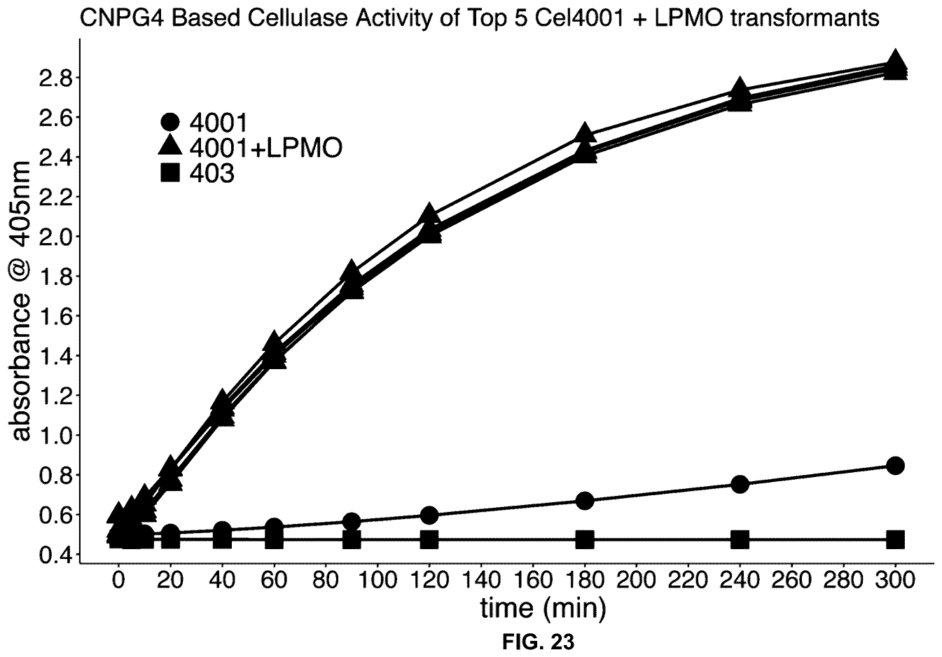

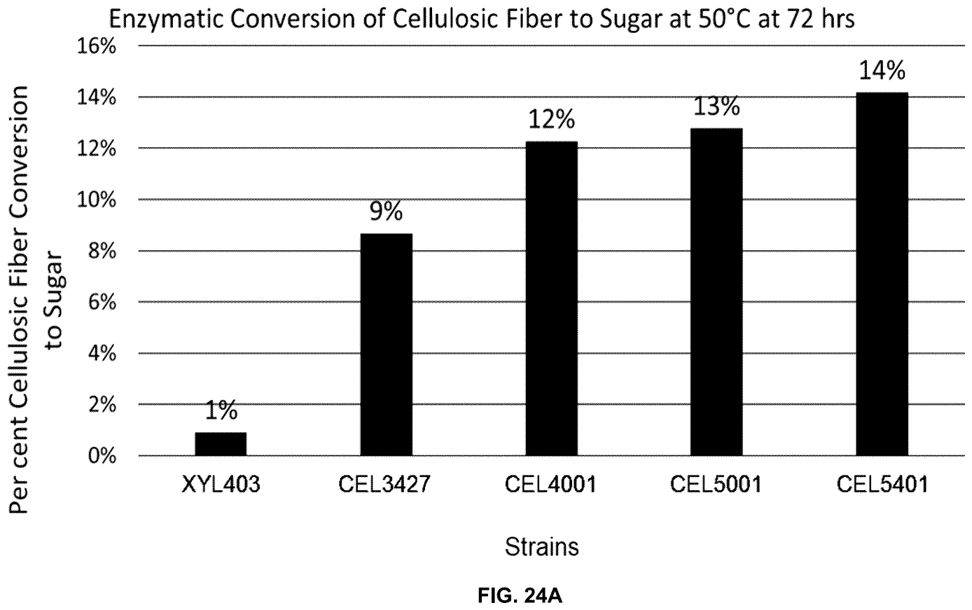

We wanted to determine whether the presence of an LPMO enzyme derived from T. reesei (TrCEL61B) and codon optimized for L. starkeyi would enhance other cellulosic enzymes when present in one of our top cellulosic strains. Since LPMOs could not we be directly assayed for activity, the activity of exo-cellobiohydrolases (CBH1 and CBHII) with and without an LPMO present were instead assayed. To do this, a CNPG4 assay was performed, which employs a very sensitive dye-labeled substrate. Results are shown in FIG. 23. The presence of an LPMO drastically improved cellulosic enzyme activity, as measured by the increase in absorbance in the strains with an LPMO compared with the base Cel4001 strain. The CEL4001 strains containing the TrCEL61B LPMO had higher absorbance, indicating high exo-cellobiohydrolase activity. The CEL4001 base strain (circles) had higher activity than XYL403 (squares, no cellulosic enzymes), but lower activity than the CEL4001 strains containing the LPMO.

### Carbohydrolytic Activities of the XYL403, CEL3427. CEL4001, CEL5001, and CEL5401 Strains on Various Fiber Sources

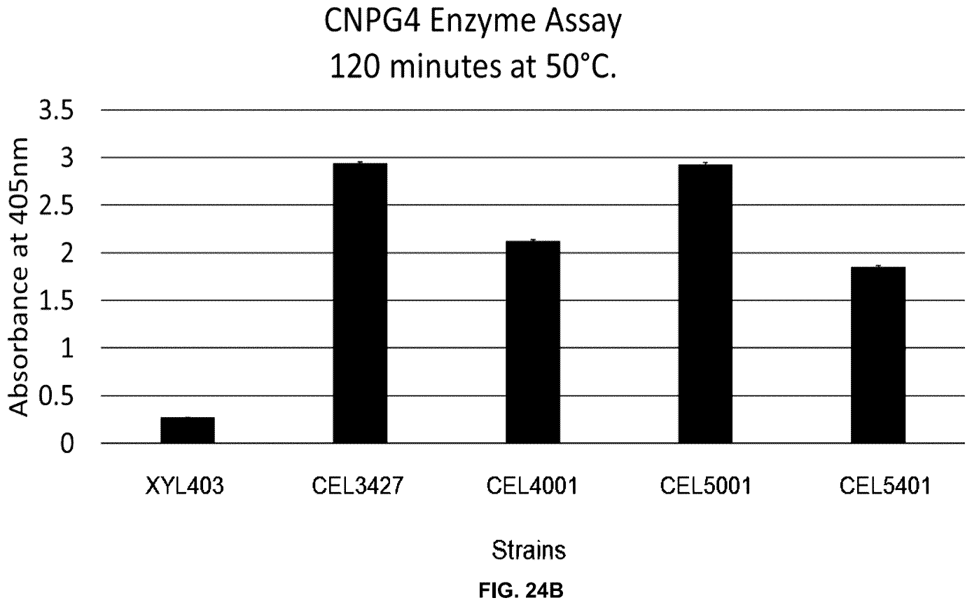

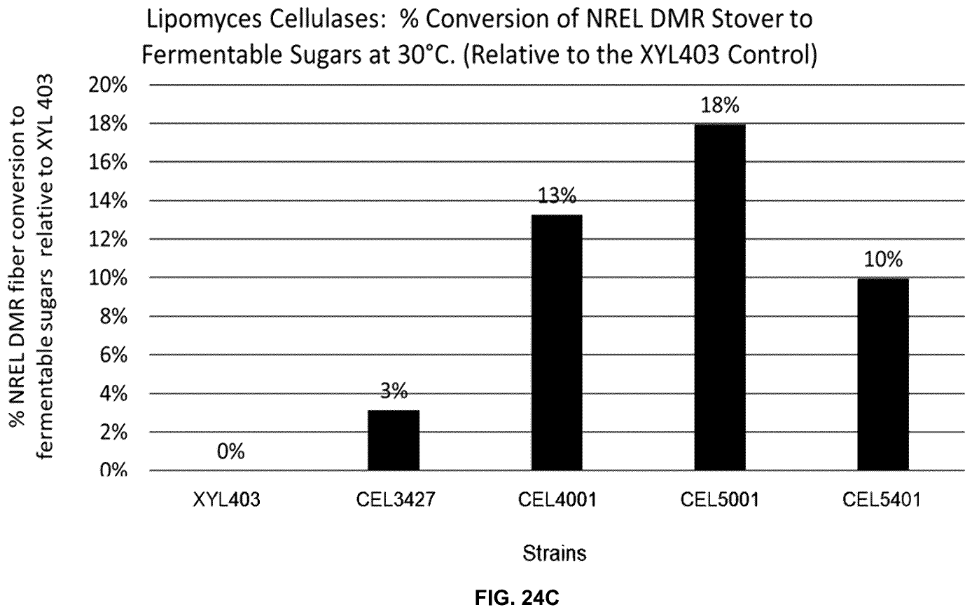

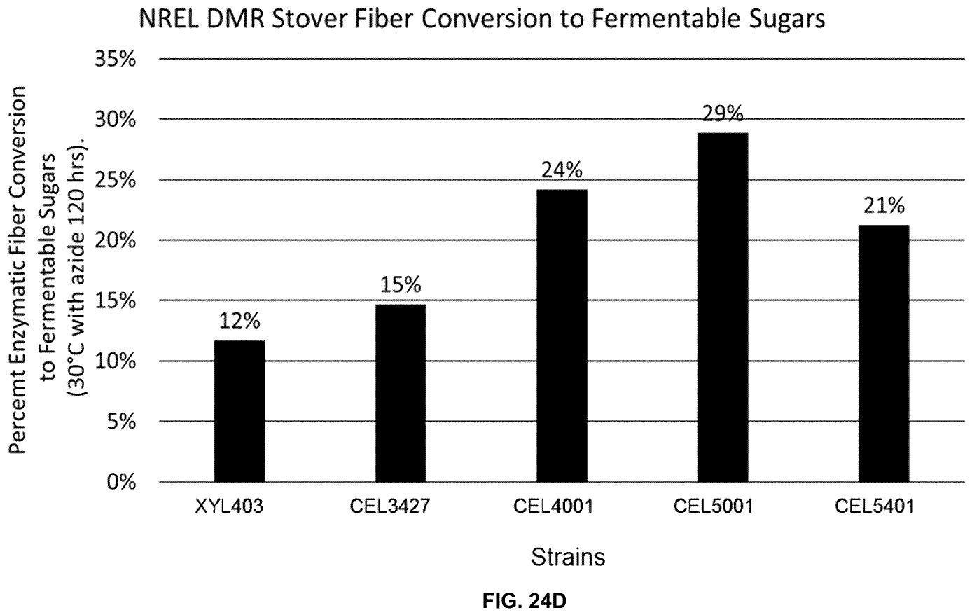

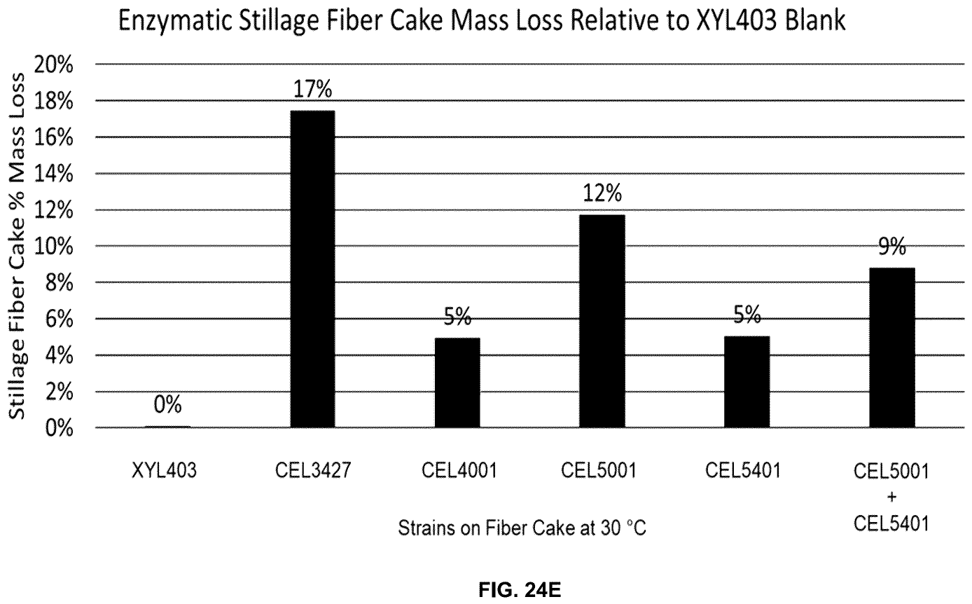

Using several fiber sources, extracellular enzyme preparations from cellulase strains of Lipomyces starkeyi (CEL3427, CEL4001, CEL5001, and CEL5401) demonstrate extracellular activities that are capable of up to 14% conversion of cellulosic fiber and 29% for a corn stover fiber into glucose and cellobiose sugars on a mass basis at 50° C. for 72 hrs (FIGS. 24A-24E). The enzyme preparation from the XYL403 control strain contains cellulosic enzymes and displays low conversion of cellulosic fiber to sugars as expected (FIGS. 24A-24E).

### REFERENCES

1 Lurty, N., Novel deployment of modem portfolio theory to achieve sustainability for dry-grind ethanol producers, in Fuel Ethanol Workshop. 2018: Omaha, NE. p. 34.
2. Hasjim, J., et al., Kernel Composition, Starch Structure, and Enzyme Digestibility of opaque-2 Maize and Quality Protein Maize. Journal of Agricultural and Food Chemistry, 2009. 57(5): p. 2049-2055.
3 Zilic, S., et al., Grain characteristics and composition of maize specialty hybrids. Spanish Journal of Agricultural Research, 2011. 9(1): p. 230-241.
4. Cook, J. P., et al., Genetic Architecture of Maize Kernel Composition in the Nested Association Mapping and Inbred Association Panels. Plant Physiology, 2012. 158(2): p. 824-834.
5. Kim, S. M., et al., A whole stillage sieving process to recover fiber for cellulosic ethanol production. Industrial Crops and Products, 2016. 92: p. 271-276.
6. Kim, Y., et al., Effect of compositional variability of distillers' grains on cellulosic ethanol production. Bioresource Technology, 2010. 101(14): p. 5385-93.
7 Fluid-Quip. FBP-Fiber By-Pass System™. 2018 [cited 2018 Jun. 19]; Available from: fqptech.com/proven-technologies/.
8. D3Max. D3Max Technology. 2018 [cited 2018 Jun. 19]; Text and Video presentation]. Available from: www.d3maxllc.com/technology.
9 POET. POET-DSM achieves cellulosic biofuel breakthrough. Press release 2017 [cited 2018 Jun. 19]; Development of a new pretreatment system enables a path for cellulosic ethanol from grain fiber. Available from: poet.com/pr/poet-dsm-achieves-cellulosic-biofuel-breakthrough.
10. Kelleher, T. J. and T. W. Jeffries, Xylome market studies and producer interviews. 2017.
11. Dugar, D. and G. Stephanopoulos, Relative potential of biosynthetic pathways for biofuels and bio-based products. Nature Biotechnology, 2011. 29(12): p. 1074-8.
12. Calvey, C. H., et al., Nitrogen limitation, oxygen limitation, and lipid accumulation in Lipomyces starkeyi. Bioresource Technology, 2016. 200: p. 780-788.
13. Probst, K. V. and P. V. Vadlani, Single cell oil production by Lipomyces starkeyi: Biphasic fed-batch fermentation strategy providing glucose for growth and xylose for oil production. Biochemical Engineering Journal, 2017. 121: p. 49-58.
14. Papanikolaou, S. and G. Aggelis, Lipids of oleaginous yeasts. Part II: Technology and potential applications. European Journal of Lipid Science and Technology, 2011. 113(8): p. 1052-1073.
15. Anschau, A., et al., Effect of feeding strategies on lipid production by Lipomyces starkeyi. Bioresource Technology, 2014. 157: p. 214-222
16. Gong, Z., et al., Co-fermentation of cellobiose and xylose by Lipomyces starkeyi for lipid production. Bioresource Technology, 2012. 117: p. 20-4.
17. Tapia, E. V., et al., Optimization of lipid production by the oleaginous yeast Lipomyces starkeyi by random mutagenesis coupled to cerulenin screening. AMB Express, 2012. 2:64.
18. USDA. Market Research and Analysis. 2018 [cited 2018 Jun. 19]; Market Research and Analysis]. Available from: www.ams.usda.gov.
19. Kruger, J. S., et al., Recovery of Fuel-Precursor Lipids from Oleaginous Yeast. ACS Sustainable Chemistry & Engineering, 2018. 6: p. 2921-2931.
20. Boulton, C. A. and C. Ratledge, Use of transition studies in continuous cultures of Lipomyces starkeyi, an oleaginous yeast, to investigate the physiology of lipid accumulation. Journal of General Microbiology, 1983. 129 (SEP): p. 2871-2876.
21. Zhao, X., et al., Medium optimization for lipid production through co-fermentation of glucose and xylose by the oleaginous yeast Lipomyces starkeyi. European Journal of Lipid Science and Technology, 2008. 110(5): p. 405-412.
22. Kim, Y., N. Mosier, and M. R. Ladisch, Process simulation of modified dry grind ethanol plant with recycle of pretreated and enzymatically hydrolyzed distillers' grains. Bioresource Technology, 2008. 99(12): p. 5177-5192.
23. Xu, Q., et al., Expression and secretion of fungal endoglucanase II and chimeric cellobiohydrolase I in the oleaginous yeast Lipomyces starkeyi. Microbial Cell Factories, 2017. 16: p. 16.
24. Shibata, N., et al., A novel GH10 xylanase from Penicillium sp. Accelerates saccharification of alkaline-pretreated bagasse by an enzyme from recombinant Trichoderma reesei expressing Aspergillus beta-glucosidase. Biotechnology for Biofuels, 2017. 10: p. 278.
25. Gilkes, N. R., et al., Domains in microbial beta-1,4-glycanases-Sequence conservation, function and enzyme families. Microbiological Reviews, 1991. 55(2): p. 303-315.
26. Liu, L. W., et al., Computational analysis of di-peptides correlated with the optimal temperature in G/11 xylanase. Process Biochemistry, 2006. 41(2): p. 305-311.
27. Wang, H., et al., Heterologous expression in Pichia pastoris and characterization of a novel GH11 xylanase from saline-alkali soil with excellent tolerance to high pH, high salt concentrations and ethanol. Protein Expression and Purification, 2017. 139: p. 71-77.
28. ICM. Thin Stillage Solids Separation System™. 2018 [cited 2018 Jun. 18]; Available from: www.icminc.com/products/thin-stillage-solids-separation-system.html.
29. Davis, R., et al., Process Design and Economics for the Conversion of Lignocellulosic Biomass to Hydrocarbons: Dilute-Acid and Enzymatic Deconstruction of Biomass to Sugars and Biological Conversion of Sugars to Hydrocarbons. 2013, National Renewable Energy Laboratory: National Renewable Energy Laboratory; 15013 Denver West Parkway Golden, CO 80401: Tel: 303-275-3000.
30. Xu, Q., et al., Expression and secretion of fungal endoglucanase II and chimeric cellobiohydrolase I in the oleaginous yeast Lipomyces starkeyi. Microbial Cell Factories, 2017. 16.
31. Nielsen, H., et al., A Brief History of Protein Sorting Prediction. The Protein Journal, 2019. 38: p. 200-216.
32. Armenteros, J. J. A., et al., SignalP 5.0 improves signal peptide predictions using deep neural networks. Nature Biotechnology, 2019. 37: p. 420-423.
33. Tomme, P., et al., Studies of the cellulolytic system of Trichoderma reesei QM 9414. European Journal of Biochemistry. 1988. 170: p. 575-581.
34. Van Tilbeurgh, H., and Claeyssens, M., Detection and differentiation of cellulase components using low molecular mass fluorogenic substrates. FEBS Letters, 1985. 187: p. 283-288.
35. Xu, Q., et al., Expression and secretion of fungal endoglucanase II and chimeric cellobiohydrolase I in the oleaginous yeast Lipomyces starkeyi. Microbial Cell Factories, 2017. 16: p. 126.
36. Van Soest, P. J. and Wine, R. H., Use of detergents in the analysis of fibrous feeds. IV. Determination of plant cell wall constituents. Journal—Association of Official Analytical Chemists, 1967. 50: p. 50.
37. Holdsworth, J. B. and Ratledge, C. Lipid turnover in oleaginous yeasts. Journal of General Microbiology, 1988. 134:339-346.
38. Lodder, J., Acomina and Kreger-Van Rij, N. J. W. The yeasts—a taxonomic study. (1952).
39. Papanikolaou, S., Chevalot, I., Komaitis, M., Aggelis, G. and Marc, I. Kinetic profile of the cellular lipid composition in an oleaginous Yarrowia lipolytica capable of producing a cocoa-butter substitute from industrial fats. Antonie Van Leeuwenhoek International Journal of General and Molecular Microbiology, 2001. 80:215-224.
40. Ratledge, C. Regulation of lipid accumulation in oleaginous micro-organisms. Biochemical Society Transactions, 2002. 30:1047-1050.
41. Holdsworth, J. E., Veenhuis, M. and Ratledge, C. Enzyme activities in oleaginous yeasts accumulating and utilizing exogenous or endogenous lipids. Journal of General Microbiology, 1988. 134:2907-2915.
42. Garay, L. A. et al. Eighteen new oleaginous yeast species. Journal of industrial microbiology & biotechnology, 2016. 43:887-900
43. Calvey, C. H., Willis, L. B. and Jeffries, T. W. An optimized transformation protocol for Lipomyces starkeyi. Current Genetics, 2014, 60:223-230.
44. Pan, L.-X. et al. Isolation of the oleaginous yeasts from the soil and studies of their lipid-producing capacities. Food Technol. Biotechnol, 2009. 47:215-220.
45. Wei, T., Sufang, Z., Qian, W., Haidong, T. and Zongbao Kent, Z. The isocitrate dehydrogenase gene of oleaginous yeast Lipomyces starkeyi is linked to lipid accumulation. Canadian Journal of Microbiology, 2009. 55:1062-1069.
46. Zhu. Z. W. et al. in Nature Communications, 2012. Vol. 3.
47. Vicente, G. et al. Direct transformation of fungal biomass from submerged cultures into biodiesel. Energy & Fuels, 2010. 24:3173-3178.

1. A recombinant yeast comprising one or more recombinant genes, wherein the one or more recombinant genes are configured to express one or more first enzymes, wherein the one or more first enzymes comprise;
any one or more of:
an exo-cellobiohydrolase I, wherein the exo-cellobiohydrolase I comprises a sequence at least 90% identical to positions 19-516 of SEQ ID NO:4 or positions 19-530 of SEQ ID NO:6;
an exo-cellobiohydrolase II, wherein the exo-cellobiohydrolase II comprises a sequence at least 90% identical to positions 25-471 of SEQ ID NO:8; and
an endoglucanase, wherein the endoglucanase comprises a sequence at least 90% identical to positions 32-254 of SEQ ID NO: 12, positions 32-344 of SEQ ID NO: 14, positions 32-441 of SEQ ID NO:16, or positions 32-428 of SEQ ID NO: 18; and
an endoxylanase, wherein the endoxylanase comprises a sequence at least 90% identical to positions 25-226 of SEQ ID NO:22, positions 25-267 of SEQ ID NO:26, or positions 25-412 of SEQ ID NO:28.

2. (canceled)

3. The recombinant yeast of claim 1, wherein the one or more first enzymes comprise an exo-cellobiohydrolase II comprising a sequence at least 90% identical to positions 25-471 of SEQ ID NO:8.

4. The recombinant yeast of claim 31, wherein the one or more first enzymes comprise an exo-cellobiohydrolase I comprising a sequence at least 90% identical to positions 19-516 of SEQ ID NO:4.

5. The recombinant yeast of claim 1, wherein the one or more first enzymes comprise an endoglucanase comprising a sequence at least 90% identical to positions 32-254 of SEQ ID NO:12, positions 32-344 of SEQ ID NO: 14, positions 32-441 of SEQ ID NO: 16, or positions 32-428 of SEQ ID NO: 18.

6. The recombinant yeast of claim 1, wherein the sequence at least 90% identical to positions 32-428 of SEQ ID NO:18.

7. The recombinant yeast of claim 1, wherein the endoxylanase comprises a sequence at least 90% identical to positions 25-226 of SEQ ID NO:22.

8. The recombinant yeast of claim 1, wherein the one or more first enzymes further comprise a lytic polysaccharide monooxygenase.

9. The recombinant yeast of claim 8, wherein the lytic polysaccharide monooxygenase comprises a sequence at least 90% identical to positions 19-247 of SEQ ID NO: 30 or positions 19-340 of SEQ ID NO:32.

10. The recombinant yeast of claim 1, wherein the one or more first enzymes further comprise an arabinofuranosidase.

11. The recombinant yeast of claim 10, wherein the arabinofuranosidase comprises a sequence at least 90% identical to a sequence selected from the group consisting of positions 27-332 of SEQ ID NO:35, positions 20-325 of SEQ ID NO: 97, positions 19-499 of SEQ ID NO: 38, positions 20-500 of SEQ ID NO:100, positions 19-499 of SEQ ID NO:40, positions 20-500 of SEQ ID NO: 103, and SEQ ID NO:42.

12. The recombinant yeast of claim 1, wherein the one or more first enzymes further comprise one or both of a xylanase and an acetylxylan esterase.

13. The recombinant yeast of claim 12, wherein:
the xylanase comprises a sequence at least 90% identical to SEQ ID NOs: 44, 46, 48, 50, or 52; and
the acetylxylan esterase comprises a sequence at least 90% identical to SEQ ID NO:54.

14. The recombinant yeast of claim 1, wherein at least one of the one or more recombinant genes encodes a secretion signal sequence operably linked to at least one of the one or more first enzymes.

15-16. (canceled)

17. The recombinant yeast of claim 1, wherein the one or more first enzymes comprise each of:
an exo-cellobiohydrolase I, wherein the exo-cellobiohydrolase I comprises a sequence at least 95% identical to positions 19-516 of SEQ ID NO:4;
an exo-cellobiohydrolase II, wherein the exo-cellobiohydrolase II comprises a sequence at least 95% identical to positions 25-471 of SEQ ID NO:8;
an endoglucanase, wherein the endoglucanase comprises a sequence at least 95% identical to positions 32-428 of SEQ ID NO: 18; and
the endoxylanase, wherein the endoxylanase comprises a sequence at least 95% identical to positions 25-226 of SEQ ID NO:22.

18. The recombinant yeast of claim 17, wherein the one or more first enzymes further comprise one or both of:
a lytic polysaccharide monooxygenase comprising a sequence at least 95% identical to positions 19-340 of SEQ ID NO:32; and
an arabinofuranosidase comprising a sequence at least 95% identical to a sequence selected from the group consisting of positions 27-332 of SEQ ID NO:35 or positions 20-325 of SEQ ID NO: 97.

19. The recombinant yeast of claim 1, wherein the one or more recombinant genes are configured to express one or more second enzymes, wherein the one or more second enzymes comprise any one or more of a diacylglycerol acyltransferase, a malic enzyme, and a glycerol-3-phosphate acyltransferase.

20-21. (canceled)

22. The recombinant yeast of claim 1, wherein the one or more recombinant genes are further configured to express one or both of a glycerol kinase and a glycerol-3-phosphate dehydrogenase.

23. (canceled)

24. The recombinant yeast of claim 1, wherein the yeast is from the genus Lipomyces.

25. (canceled)

26. A method of processing a substrate comprising contacting the substrate comprising a first organic with the yeast of claim 1, wherein the yeast consumes the first organic and produces a second organic.

27. The method of claim 26, wherein the first organic comprises one or more of cellulose, hemicellulose, and glycerol and the second organic comprises lipid.

28. (canceled)
